(19) United States
(12) Patent Application Publication
(10) Pub. No.: US 2018/0258495 A1
(43) Pub. Date: Sep. 13, 2018

(54) METHOD TO DETECT COLON CANCER BY MEANS OF THE MICROBIOME

(71) Applicants: Regents of the University of Minnesota, Minneapolis, MN (US); University of Nantes, Nantes (FR)

(72) Inventors: Dan Knights, St. Paul, MN (US); Ran Blekhman, Minneapolis, MN (US); Emmanuel Montassier, Nantes (FR)

(21) Appl. No.: 15/947,366

(22) PCT Filed: Oct. 6, 2016
(86) PCT No.: PCT/US2016/055758

§ 371 Date: Apr. 6, 2018

Related U.S. Application Data
(60) Provisional application No. 62/237,923, filed on Oct. 6, 2015.

(51) Int. Cl. C12Q 1/6886; C12Q 1/689

(52) U.S. Cl. CPC C12Q 1/6886; C12Q 1/689

(57) ABSTRACT

A method and kit to detect colon cancer in a human by detecting the presence or absence, or relative amount (abundance) of, certain bacteria in a sample, is provided.

### CROSS-REFERENCE TO RELATED APPLICATIONS

This application claims the benefit of the filing date of U.S. application Ser. No. 62/237,923, filed on Oct. 6, 2015, the disclosure of which is incorporated by reference herein.

### BACKGROUND

Colorectal cancer (CRC) is the second most commonly diagnosed cancer in females and the third in males worldwide (Jemal et al., 2011). The microbial communities present in the intestinal tract have known associations with colon health, though until recently researchers were limited to the study of microbes that were amenable to in vitro culturing. As a result of recent advances in culture-independent measurements of microbial communities, we know that the human gut is host to roughly a thousand different bacterial species (The Human Microbiome Consortium, 2012). Alterations of this bacterial community are correlated with host health, including diseases ranging from diabetes and obesity to Crohn's disease and arteriosclerosis (Jones at al., 2014). The composition of the gut microbiome also has a known association with CRC, although the direction of causality remains unclear (Konstantinov et al., 2013; Sobhani at al., 2011; Bonnet et al., 2014; Castellarin et al., 2012; Tahara et al., 2014; Chen et al., 2012; Kostic et al., 2012). A recent report demonstrated that analysis of the microbiome can be used as a pre-screening test for CRC that dramatically outperforms the current standard methods (Zackular et al., 2014). These analyses have identified significant shifts in the relative abundances of specific bacterial taxa in CRC cancer patients' colon mucosa and stool microbiomes. For instance, bacteria in the genus Fusobacterium are enriched in some CRC patients' microbiomes (Castellarin et al., 2012; Tahara et al., 2014; Kostic et al., 2012; Marchesi et al., 2011). Fusobacterium are thought to elicit a pro-inflammatory microenvironment around the tumor, driving tumor formation and/or progression (Castellarin et al., 2012). More specifically, a recent study has demonstrated that the FadA protein, a virulence factor expressed by Fusobacterium nucleatum, can signal epithelial cells via E-cadherin, a cell-surface molecule important for CRC metastasis as well as a component of the WNT/β-catenin signaling pathway, the most commonly mutated pathway in CRC (Cancer Genome Atlas Network, 2012; Rubinstein et al., 2013). Other cancer-associated bacterial taxa have been identified, including Escherichia coli strain NC101 and Bacteroides fragilis, each with a proposed mechanism of interaction with colon cancer (Irrazábal et al., 2014). The species Akkermansia mucinphila, a bacterium that has known associations with obesity, has also been implicated as a cancer-associated agent, with its mucin-degrading activity as a proposed mechanism to drive inflammation contributing to cancer genesis and/or progression (Irrazábal et al., 2014).

In addition to defining the set of bacteria significantly associated with CRC, several groups have used measurements of microbiome diversity to compare cancer patients with normal subjects. There are distinct differences in these results that depend on the sources of the samples used to assess the microbiome (e.g., stool samples versus mucosal or tissue samples, longitudinal versus cross-sectional sampling). For instance, in a study that used stool samples to compare CRC patients with normal controls, the researchers showed decreased alpha-diversity among the microbial communities found in the CRC patients' stools compared with the control (Ahn et al., 2013). Another, recent study, also focusing on fecal samples, was unable to detect differences in microbial community diversity or richness between normal and cancer-associated microbiomes (Zeller et al., 2014). However, in a study that used tissue samples from patients with colon adenomas and compared them with patient-matched normal tissues, the alpha-diversity present at the site of the lesion was actually increased (Shen et al., 2010). This finding was repeated by Mira-Pascual et al. (2014), who performed side-by-side analyses of tissue and stool samples. They found that stool samples in general had roughly twice the microbial diversity when compared with tissue-associated microbiomes, with stools from cancer patients showing lower diversity than those from normal controls. When comparing the microbial diversity between tissue samples, however, the tumor microbiome was more diverse than the normal microbiome. It is likely that this is a function of the stool samples harboring microbes from the entire colonic environment, including species that are not directly related to the tumor microenvironment, adding noise to the taxonomic results acquired from assessment of stool samples relative to direct measurements of tissues. These findings suggest that in order to detect differences specific to the cancer-associated microbiome, samples taken directly from the tumor microenvironment are preferable, at least at the initial characterization phase, to bulk stool samples, which are not likely to have the discriminatory power required to measure small, yet significant, effects (Mira-Pascul et al., 2014).

DNA sequencing of microbial genes in the stool micro biota has already been shown (Zackular et al., 2014) to be a somewhat effective way to detect the presence of colon cancer without the need for invasive colonoscopy. However, there are currently no methods for detecting or predicting the specific types of mutations present in the tumor genome apart from sequencing the exome or genome of tumor cells.

### SUMMARY

Microbiome and somatic tumor genetic variation were jointly analyzed. A combined approach of whole-exome sequencing and microbiome profiling was used in 44 tumor biopsies and 44 normal colonic tissue samples from the same individual. Using these data, a strong concordance was found between tumor mutational patterns and shifts in the microbiome. The number of loss-of-function mutations in tumors was correlated with the diversity of the tumor's microbiota, whereby hypermutated tumors had a significantly more diverse microbiome. Moreover, somatic mutations in certain genes were correlated with changes in abundance of specific microbial taxa. A risk index from a panel of microbes correlated with each of several prevalent tumor-driving mutations. These results highlight a significant and clear interaction between the genetic profile of colorectal tumors and the composition of the tumor-attached microbiome. These results serve as a starting point for colon cancer prognostics and treatments that target and use the microbiome. For example, microbial profiles fall into different groups; one group for those tumors with a normal gene, e.g., a normal KMT2C gene, and one group for the microbial profiles for tumors with a LoF mutation in the gene. Those taxa that are associated with the mutation are those that are increased in the tumors with the mutation relative to those that no not have the mutation. The taxa that are associated with “no mutation” are those that decrease in the tumors with the mutation relative to those tumors that lack the mutation. Thus, the methods provide for a determination of whether a patient has colon cancer and which genes in the tumor may be mutated, e.g., based on any change in abundance of specific bacteria.

Thus, this disclosure describes a method for predicting whether certain types of mutations are present in a particular tumor genome. e.g., in a sample from a mammal such as a human, using only the taxonomic profile of the microbial community living in the tumor microenvironment. In one embodiment, the method allows for prediction of whether a cancer has loss-of-function (LOF) mutations in the human genes APC, ANKRD36C, CTBP2, or KMT2C, significantly better than random (permutation test, false-discovery-rate-corrected p<0.05). There is also evidence suggesting that this method predicts ZFN717 loss-of-function mutations better than random (p-value=0.06 by permutation test after False Discovery Rate (FDR) correction for multiple tests). The benefit of associating specific bacterial community profiles with tumor genetic mutations is that: (1) if that particular tumor mutation types are related to specific bacterial taxa, then different subtypes of microbial communities may be utilized in stool-based colon cancer screens that likely would have a stronger predictive capability than current methods that rely on a single overarching colon cancer “type;” and (2) non-invasive stool-based colon cancer screens may be used to predict the presence of specific types of mutations in a colon cancer.

In one embodiment, a method to detect colon cancer or a risk of colon cancer in a human is provided. The method includes providing a stool sample or tissue biopsy sample, e.g., from the large intestine, from a human and detecting in the sample the presence or absence, or relative amount (abundance) of, bacteria including one or more of Actinobacteria OPB41; Saprospiroceae; Flavobactenaceae, e.g., Capnocytophaga; Christensenellaceae; phylum OD1 class ABY1; Chthoniobacteraceae DA101; Addobacteria BPC102, 8110; Acidomicrobiates; Corynebacterium; Collenesia; Rhodocyclaceae; phylum TM7 class TM7_1; Thermogymnomonas, Cryomorphaceae, Gemmatimonadetes, Sphingomonadaceae, Moraxellaceae; class Verruco_5 order SS1B 03 39; Haptophyceae, Alphaproteobacteria; Rhizobiaceae; or Chromatialis. In one embodiment, the presence of, or an increase in the relative amount of, one or more bacteria, e.g., relative to the amount in a control sample, relative to one or more bacteria in the sample, for instance, bacteria which is/are correlated with colon cancer or an increased risk of colon cancer to those which is/are not correlated with colon cancer or an increased risk of colon cancer, is indicative of an increased risk of colon cancer in the human. In one embodiment, the increase in the amount of a plurality of bacteria associated with colon cancer, e.g., relative to other bacteria in the sample including those that are not correlated with colon cancer. In one embodiment, the presence of, or an increase in the amount of, the one or more bacteria, e.g., relative to a control sample or relative to one or more different bacteria, e.g., those where the presence of which is/are not correlated with an increased risk of colon cancer, is indicative of a decreased risk of colon cancer or the absence of colon cancer in the human. In one embodiment, the specified bacteria are detected using a nucleic acid amplification reaction. In one embodiment, the specified bacteria are detected by detecting 16S rRNA, e.g., the specified bacteria are detected by detecting 16S rRNA at more than one taxonomic level. In one embodiment, the specified bacteria are detected using DNA sequencing, e.g., whole genome sequencing. In one embodiment, detecting includes hybridizing bacterial nucleic acid, e.g., amplified bacterial nucleic acid, to beads or an array. In one embodiment, a plurality of the specified bacteria are detected, for instance, at the same time.

In one embodiment, a method to predict or detect a loss of function (LoF) mutation in an adenomatous polyposis coli (APC) gene, zinc finger nuclease 717 (ZFN717) gene, ankyrin repeat domain (ANKR1) gene, C-terminal binding protein (CTPB2) gene, or lysine N-methyltransferase 3C (KMT2C) gene in a human is provided. The method: providing a biopsy sample, e.g., from the colon, or a stool sample from a human and detecting in the sample the presence or absence, or relative amount of, bacteria including one or more of Actinabacteria OPB41; Saprospiroceae; Flavobacteriaceae, e.g., Capnocytophaga; Christensenellaceae phylum OD1 class ABY1; Chthoniobacteraceae DA101; Aadobacteria BPC102, 8110; Acidomicrobiates; Corynbactenum; Collenesia; Rhodocycdaceae; phylum TM7 class TM7_1; Chthoniobactereceae DA101; Thermogymnomonas, Cryomorphaceae, Gemmatimonadetes, Sphingomonadaceae, Moraxellaceae; class Verruco_5 order SS1B 03 39; Haptophyceae. Alphaproteobacteria; Rhizobiaceae; or Chromatialis. In one embodiment, the presence or the amount of the one or more bacteria is indicative of a LoF mutation in APC, ANKR1, ZFN717, CTPB2, or KMT2C. In one embodiment, the specified bacteria are detected using a nucleic acid amplification reaction. In one embodiment, the presence or the amount of the one or more bacteria is indicative of a decreased risk of a LoF mutation in APC, ANKR1, ZFN717, CTPB2, or KMT2C. In one embodiment, the specified bacteria are detected using 16S rRNA. In one embodiment, the specified bacteria are detected using sequencing, e.g., whole genome sequencing. In one embodiment, detecting includes hybridizing bacterial nucleic acid, e.g., amplified bacterial nucleic acid, to beads or an array. In one embodiment, a plurality of the specified bacteria are detected, for instance, at the same time, from the same sample and/or in one sample (multiplexing).

Further provided is a kit or panel comprising: a set of probes to detect, or a set of primers to amplify sequences specific for, one or more of Actinobacteria OPB41; Saprospirocea Flavobactedaceae, e.g., Capnocytophaga; Christensenellaceae; phylum OD1 class ABY1; Chthoniobacteraceae DA101; Aadobacteria BPC102, B110; Acidomicrobiates; Corynbacterium; Collenesia; Rhodocydaceae; phylum TM7 class TM7_1; Chthoniobactereceae DA101; Thermogymnomonas, Cryomorphaceae, Geminatimonacetes, Norosphingobium, Moraxelladeae; class Verruco_5 order SS1B 03 39; Haptophyceae, Alphaproteobacteria; Rhizobiaceae; or Chromatialis. In one embodiment, the probes or primers detect 16S rRNA, e.g., V5-V6 of 16S rRNA.

### BRIEF DESCRIPTION OF THE FIGURES

FIG. 1. Genes differentiated by taxa.

FIG. 2. Risk indices for select genes.

FIG. 3. Bacterial taxa associated with loss-of-function mutations (or the absence of loss-of-function) for 5 different genes. This represents taxa that were found in all the LOO for one gene.

FIG. 4. Genes and bacterial taxa analyzed. This represents the taxa differentiating mutation versus no mutation in the whole dataset, in the 44 patients, without any LOO procedure.

FIGS. 5A-C. Correlation between the microbial community at a tumor that differentiates between tumor stage. A) Low-stage (stages 1-2) and high-stage (stages 3-4) tumors can be differentiated using a risk index classifier generated from microbial abundance data (y-axis). The central black bar indicates the median, and the thin black bars represent the 25th and 75th percentiles. B) A receiver operating characteristic (ROC) curve was generated using a 10-fold cross-validation (blue dotted lines). The average of the 10-fold cross-validation curves is represented as a thick black line. C) Differences in the mean abundances of a subset of the taxa predicted to interact differentially with high-stage and low-stage tumors. This subset represents those taxa that had a mean difference in abundance of greater than 0.1%, proportionally.

FIGS. 6A-D. Commonly mutated genes show a predicted interaction with changes in the abundances of several microbial taxa. A) A heatmap of the scaled abundances values (cells) for a subset of taxa chosen as they were identified as discriminatory in each leave-one-out iteration (columns) that were found significantly associated with prevalent LoF mutations (rows). Scaled abundances are from the patients with the indicated mutations. B) LoF mutations in each of the indicated genes can be predicted using a risk index as a classifier (y-axis). The central black bar indicates the median, and the thin black bars represent the 25th and 75th percentiles. C) ROC curves were generated for each of the indicated mutations using a 10-fold cross-validation (blue dotted lines). The average of the 10-fold cross-validation curves is represented as a thick black line. D) Differences in the mean abundances of a subset of the taxa predicted to interact differentially with tumors with a LoF mutation relative to those without the indicated mutation. This subset represents those taxa that had a mean difference in abundance of greater than 0.1%, proportionally.

FIGS. 7A-H. Pathways harboring prevalent LoF mutations correlate with changes in the abundances of sets of microbial taxa. A) A heatmap of the scaled abundances values (cells) for a subset of taxa (columns) that are found significantly associated with KEGG pathways harboring LoF mutations (rows). Scaled abundances are from the patients with mutations in the indicated pathways. B) LoF mutations in each of the indicated pathways can be predicted using a risk index as a classifier (y-axis). The central black bar indicates the median, and the thin black bars represent the 2nd and 4th quartiles. C) ROC curves were generated for each of the indicated pathways using a 10-fold cross-validation (blue dotted lines). The average of the 10-fold cross-validation curves is represented as a thick black line. D) Differences in the mean abundances of a subset of the taxa predicted to interact differentially with tumors harboring mutations in the indicated pathways relative to those without a mutation. This subset represents those taxa that had a mean difference in abundance of greater than 0.1%, proportionally. E-H) Identically structured visualizations as in A)-D), but for PID pathway data rather than the KEGG pathways.

FIGS. 8A-B. Interaction networks among bacteria are defined by host tumor mutations. A) SparCC analysis of the microbial abundances for taxa identified by LEfSe for APC with LoF mutations (left) and without mutation (right) produce distinct patterns of correlations (edges) between a common set of taxa (nodes). Direct correlations are indicated as red edges and inverse correlations as blue edges (SparCC R>=0.25, P<=0.05 for displayed edges). B) SparCC analysis was run simultaneously for all taxa identified by LEfSe when predicting PID pathways. There are interactions (dashed edges) between the taxa (grey nodes) associated with mutations across sets of PID pathways (green nodes). The solid edges indicate SparCC R-values (red for direct and blue for inverse correlations). The grey taxon nodes are scaled to the average abundance of the taxa in the associated tumor set. Edge color indicates the direction of the interaction, red for negative and blue for positive.

FIGS. 9A-B. Heatmaps demonstrating the patterns of microbial abundances for patient samples with prevalent LoF mutations. A) Scaled taxon abundances (columns) in the tumor samples that harbor LoF mutations in the genes indicated (rows). B) Scaled differences (tumor abundance-matched normal abundance) patients that harbor tumor-specific LoF mutations in the genes indicated (rows).

FIGS. 10A-B. Heatmaps demonstrating the patterns of microbial abundances for patient samples with prevalent LoF mutations in KEGG pathways A) Scaled taxon abundances (columns) in the tumor samples that harbor LoF mutations in the KEGG pathways indicated (rows). Clusters 1 and 2 are labeled to facilitate discussion in main text. B) Scaled differences (tumor abundance−matched normal abundance) patients that harbor tumor-specific LoF mutations in the KEGG pathways indicated (rows).

FIGS. 11A-B. Heatmaps demonstrating the patterns of microbial abundances for patient samples with prevalent LoF mutations in PID pathways. A) Scaled taxon abundances (columns) in the tumor samples that harbor LoF mutations in the PID pathways indicated (rows). B) Scaled differences (tumor abundance−matched normal abundance) patients that harbor tumor-specific LoF mutations in the PID pathways indicated (rows).

FIG. 12. LoF mutations in KEGG pathways can be predicted using a risk index as a classifier (y-axis).

FIG. 13. ROC curves were generated for each of the KEGG pathways indicated in FIG. 12 using a 10-fold cross-validation (blue dotted lines). The average of the 10-fold cross-validation curves is represented as a thick black line. The AUC are indicated at bottom of each pathway panel.

FIG. 14. LoF mutations in PID pathways can be predicted using a risk index as a classifier (y-axis).

FIG. 15. ROC curves were generated for each of the PID pathways indicated in FIG. 14 using a 10-fold cross-validation (blue dotted lines). The average of the 10-fold cross-validation curves is represented as a thick black line. The AUC are indicated at bottom of each pathway panel. This figure also includes BARD1 and Class I PI3K pathways, for references, although neither of these achieved statistical significance in other tests.

FIGS. 16A-C. Abundance bar charts. A) Genes. B) KEGG. C) PID.

FIG. 17. Patient and tumor metadata for the samples in this study as well as those used in the study Burns et al., 2015. na=not available; MSI=microsatellite instability; MSS=microsatellite stable

FIG. 18. Different sets of microbes correlate with different tumor stages. A listing of the microbial taxonomies and their correlations with High (3-4) or Low (1-2) stage.

FIG. 19. Different sets of microbes correlate with LoF mutations. A listing of the microbial taxonomies and the direction of their correlations with LoF mutations the indicated genes. All taxa listed were identified using LefSe and had LDA (log 10) scores above 2.

FIG. 20. Differences in taxonomic abundances can be used to predict the presence of prevalent LoF mutations. Summary of the mutated genes, KEGG, and PID pathways and the accuracy and significance (Q-values obtained by FDR-correction of P-values) with which they can be predicted using the abundances of discriminatory microbial taxa.

FIG. 21. Different sets of microbes correlate with LoF mutations in KEGG pathways. A listing of the microbial taxonomies and the direction of their correlations with LoF mutations the indicated KEGG pathways All taxa listed were identified using LefSe and had LDA (log 10) scores above 2.

FIG. 22. Different sets of microbes correlate with LoF mutations in PID pathways. A listing of the microbial taxonomies and the direction of their correlations with LoF mutations the indicated PID pathways All taxa listed were identified using LefSe and had LDA (log 10) scores above 2.

### DETAILED DESCRIPTION

The use of traditional case-control studies of the colon cancer microbiome makes it difficult to control for all of the external effects on the microbiome. For example, the composition of gut microbial communities is strongly affected by diet (David et al., 2014). Host genetic variation is also expected to control variation in the gut microbiome (Cullender et al., 2013), through differences in host immune response among other genetic mechanisms (Knights et al., 2013). The large effects these factors have on microbiome composition are likely to confound traditional case-control studies. By using tumor and normal tissue samples taken from the same individual, our study controls for these variables internally, providing a more accurate view of the tumor-associated shifts in the microbiome. Several previous studies have utilized this strategy to assess changes in the cancer-associated microbiome in a variety of populations (Castellarin et al., 2012; Chen et al., 2012; Kostic et al., 2012; Marchesi et al., 2011; Mira-Pascual at al., 2014; Geng at al., 2013; Dejea at al., 2014). However, most used far fewer samples than would be necessitated by using unmatched tissue or stool samples. Another caveat with these analyses, even with the use of patient-matched samples, is that the normal tissue itself may possibly be affected by the changes elicited in the organ by the tumor. There has been a report indicating that, in some cases, biofilms may form in otherwise normal areas of the colon of patients who also have tumors (Dejea et al., 2014). In the event that this is the case, the overall differences seen between tumor and normal matched tissue microbiomes might be diminished as the tumor is producing a “field effect” that influences surrounding tissue.

Changes in the normal microbial communities living in the colon have been associated with incidence of colon cancer. In our recently published work (Burns et al. Genome Medicine 2015) we showed that this is also true in the tumor microenvironment. DNA sequencing of microbial genes in the stool micro biota has already been shown (Zackular et al. Cancer Prev Res 2014) to be a somewhat effective way to detect the presence of colon cancer without the need for invasive colonoscopy. However, there are currently no methods for detecting or predicting the specific types of mutations present in the tumor genome apart from sequencing the exome or genome of tumor cells. The benefit of associating specific bacterial community profiles with tumor genetic mutations would be twofold: (1) the discovery that particular tumor mutation types are related to specific bacterial taxa would imply that utilizing different subtypes of microbial communities in stool-based colon cancer screens would likely have a stronger predictive capability than the current methods that rely on a single overarching colon cancer “type;” and (2) it would be possible that non-invasive stool-based colon cancer screens could be used to predict the presence of specific types of mutations in a colon cancer.

### Exemplary Methods to Detect Taxa in a Tumor Microenvironment

### Tissue Samples and DNA Isolation

88 tissue samples from 44 individuals were used, with one tumor and one normal sample from each individual. These de-identified samples were obtained from the University of Minnesota Biological Materials Procurement Network (Bionet), a facility that archives research samples from patients who have provided written, informed consent. All research conformed to the Helsinki Declaration and was approved by the University of Minnesota Institutional Review Board. Tissue pairs were resected concurrently, rinsed with sterile water, flash frozen in liquid nitrogen, and characterized by staff pathologists. The criteria for selection were limited to the availability of patient-matched normal and tumor tissue specimens. The specific site of the tumor within the intestinal tract was recorded. Total DNA was isolated from the flash-frozen tissue samples and their associated microbiomes by adapting an established nucleic acid extraction protocol (Burns et al., 2013). Briefly, approximately 100 mg of flash-frozen tissue were physically disrupted by placing the tissue in 1 mL of Qiazol lysis solution and sonicating in a heated (65° C.) ultrasonic water bath for 1-2 hours. The efficiency of this approach was verified by observing high abundances of Gram-negative bacteria across all samples, including those from the phylum Firmicutes. Additionally, sequences from the notoriously difficult to lyse bacterial genera Mycobacterium and/or Bacillus (Vandeventer et al., 2011) were detected in the majority of samples, also indicating a rigorous and efficient lysis. DNA was purified from the lysate using the Qiagen All-prep kit (Qiagen Inc., Valencia, Calif., USA).

16S rRNA Sequencing

Briefly, DNA isolated from colon samples was quantified by quantitative PCR (qPCR), and the V5-V6 regions of the 16S rRNA gene were PCR amplified with the addition of barcodes for multiplexing. The forward and reverse primers were the V5F and V6R sets from Cai et al. (2013). The PCR conditions were as follows. Amplification was carried out in a 25 μL PCR reaction with 5 μL of template DNA with an initial denaturation step at 95° C. for 5 minutes followed by 30 cycles of denaturation (50 seconds at 94° C.), annealing (30 seconds at 40° C.), and elongation (30 seconds at 72° C.). Amplified samples were then diluted 1:100 in water for input into library tailing PCR. This PCR reaction was similar to initial amplification except the PCR conditions consisted of an initial denaturation at 95° C. for 5 minutes followed by 15 cycles of denaturation (50 seconds at 94° C.), annealing (30 seconds at 40° C.), and elongation (1 minute at 72° C.). Quantification of PCR products was performed using the Quant-iT PicoGreen dsDNA Assay Kit (Life Technologies, Grand Island, N.Y., USA). A subset of the amplified libraries was spot-checked on a Bioanalyzer High-Sensitivity DNA Chip (Agilent Technologies, Santa Clara, Calif., USA) to ascertain if the amplicons were the predicted size. These samples were each normalized to 2 nM and pooled. The total volume of the libraries was reduced using a SpeedVac and amplicons were size-selected at 420 bp±20% using the Caliper XT (Perkin Elmer, Waltham, Mass., USA). The pooled libraries were cleaned with 1.8×AMPureXP beads (Beckman Coulter, Brea, Calif., USA) and eluted with water. DNA concentration in the final pool was assayed with PicoGreen and normalized to 2 nM for input into Illumina MiSeq (v3 Kit) to produce 2×250 bp sequencing products. Clustering was performed at 10 pM with a 5% spike of PhiX. A single lane on an Illumina MiSeq instrument was used to generate the 16S rRNA gene sequences. Raw sequencing data have been submitted to the NCBI Sequence Read Archive under project accession PRJNA284355.

PCR and qPCR

Quantitative real-time PCR was performed to assess the abundance of the FadA gene present in a subset of normal and tumor tissue pairs. DNA from the ATCC control strain of F. nucleatum 25586 was used as a positive control. FadA abundances were normalized relative to paneubacteria abundance per sample. Primers FadA-F (5′-GAAGAAAGAGCACAAGCTGA-3′; SEQ ID NO:1) and FadA-R (5′-GCTTGAAGTCTTTGAGCTCT-3′; SEQ ID NO:2) were used to measure FadA (Rubinstein et al., 2013), and primers for universal eubacteria 16S (5′-GGTGAATACGTTCCCGG-3′; SEQ ID NO:3) and (5′-TACGGCTACCTTGTTACGACTT-3′; SEQ ID NO:4) were used to determine the total eubacterial abundance per sample (Kostic et al., 2013). The analysis was performed using 10 ng of DNA in a 20 μL reaction containing FastStart Universal SYBR Green Master Mix (Rox; Roche Diagnostics, Indianapolis, Ind., USA) on an Applied Biosystems 7300 Real Time PCR system. Reactions were performed in triplicate. FadA relative abundances were calculated as per the ΔCT method (Pfaffl, 2001). Relative fold differences were calculated by dividing the FadA abundance from the normal samples by that of the tumor sample.

Fusobadcterium genus-specific PCR was performed on a subset of samples using previously characterized primers: forward (5′-GGATTTATTGGGCGTAAAGC-3′; SEQ ID NO:5) and reverse (5′-GGCATTCCTACAAATATCTACGAA-3′; SEQ ID NO:6) (Kostic et al., 2013; VBoutaga at al., 2005). The PCR was carried out using Accustart Taq polymerase (Quanta Biosciences, Gaithersburg, Md., USA) following the manufacturer's protocol for 30 cycles with an annealing temperature of 55° C. DNA from the ATCC control strain F. nucleatum 25586 was used as a positive control.

Providencia genus-specific PCR was performed using a previously published protocol and primer set: sp16s-F1 (5′-ACCGCATAATCTCTTAGG-3′; SEQ ID NO:7) and Psp16s-R2 (5′-CTACACATGGAATTCTAC-3′; SEQ ID NO:8), with the following modifications (SHima et al., 2012). The PCR was carried out using Accustart Taq polymerase (Quanta Biosciences, Gaithersburg, Md., USA) following the manufacturer's protocol for 30 cycles with an annealing temperature of 50° C. The ATCC control strain Providencia alcalifaciens 9886 was used as a positive control. Amplicons were resolved in 2% agarose TAE gel.

### Sequence Analysis

The sequence data contained approximately 21.4 million reads passing quality filtering in total, inclusive of forward and reverse reads, with a mean value of 242,940 quality reads per sample.

The forward and reverse read pairs were merged using the USEARCH v7 program ‘fastq_mergepairs’, allowing stagger, with no mismatches allowed (Edgar, 2010). Merged reads were quality trimmed, again using USEARCH, to truncate reads at any quality scores of 20 or less. Following merging and trimming, there were an average of 62,100 high quality reads per sample (median 48,817; range 6559-173,471). The fasta sequence headers were renamed using a custom script to conform to QIIME standards.

The merged and filtered reads were used to pick operational taxonomic units (OTUs) with QIIME v.1.7.0 using ‘pick_otus.py’, with the closed-reference usesearch_ref OTU picking protocol against the Greengenes database (August 2013 release) at 97% similarity (Caporaso et al., 2010; Navas-Molina et al., 2013; DeSantis et al., 2006). Reverse read matching was enabled, while reference-based chimera calling was disabled. Rarefaction was performed on the OTU table at 5000 reads prior to subsequent analyses.

The final OTU table was used to generate a phylogenetic tree by including only taxa with at least 0.1% relative abundance in at least half of all samples. Starting with the full reference tree provided by the Greengenes database (August 2013 release, file 97_otus_unannotated.tree), a smaller tree file that contained only this limited set of taxa was generated using a custom pipeline (Sycamore from the Aim laboratory at MIT). The output of this pipeline was visualized with the Interactive Tree of Life (DeSantis et al., 2006; Letunic and Bork, 2011). See Additional file 2 for the OTU table used in this study.

A linear model was used to correct for several patient and tumor covariates, individually as well as in combination, including patient age, sex, tumor stage, and tumor site. None of these factors, alone or in combination, were found to have a significant impact in this sample set. Principle coordinate analysis was performed using the difference between the tumor and normal abundances for each taxon. Using this unsupervised approach, there was no clear segregation of the patients by age, sex, tumor stage, site, or microsatellite instable (MSI) status. Additionally, Providencia and Fusobacterium, were specifically focused on and while there was a slight trend toward higher tumor stage with increases in these two genera at the tumor site, it was not statistically significant. Microsatellite instable/microsatellite stable (MSI/MSS) statuses were only available for 13 of the 44 patients.

Correlation analysis was performed using SparCC, available from Jonathan Friedman at MIT, on the complete OTU table collapsed to the genus level (Friedman and Aim, 2012). Pseudo p values were inferred using 100 randomized sets. Correlations with pseudo p values≤0.05 that were within two degrees of separation from Providencia or Fusobacterium with absolute correlations of 0.05 or more were visualized using Cytoscape v.3.1.0 (Lopes et al., 2010).

The PICRUSt v.1.0.0 pipeline was used to generate a virtual metagenome using the OTU table containing raw counts generated in the previous analyses by QIIME (Langille, et al., 2013; Caporaso et al., 2010; Navas-Molina at al., 2013). Pathways and enzymes were assigned using the Kyoto Encyclopedia of Genes and Genomes (KEGG) database options built into the pipeline. Virulence genes were identified by mapping the data in the PICRUSt enzyme abundance table to MVirDB using the UniProt database file, idmapping.dat, as a key.

### Results

Tumor Microenvironments Harbor Microbiomes Distinct from Those of Normal Tissue

### Microenvironments

Patient-matched normal and tumor colon tissue samples were obtained from the University of Minnesota Biological Materials Procurement Network (BioNet) from 44 patients. The microbiome associated with each sample was assessed by Illumina sequencing across the V5-V6 hypervariable regions of the 16S rRNA gene (see “Methods” for details). This analysis showed variation in the bacterial phyla abundance when comparing the matched normal and tumor tissues. This variability is consistent with previous reports and demonstrates that there is indeed a cancer-associated signature in the tumor microbiome (Bonnet et al., 2014; Kostic et al., 2012; Ahn et al., 2013; Shen et al., 2010; Kostic et al., 2013; Arthur et al., 2012; Wu et al., 2013). At the level of the phyla, each sample was dominated by Firmicutes, Bacteroidetes, and Proteobacteria. There were clear and significant changes in these phyla between the normal and cancer states, with the tumors showing an enrichment of Proteobacteria and a depletion of Firmicutes and Bacteroidetes. Also consistent with previous reports, an increase in the phylum Fusobacteria was observed in the tumor-associated microbiome (two-sided Wilcoxon signed rank test q≤0.1 after false discovery rate (FDR) correction for multiple tests) (Castellarin et al., 2012; Kostic et al., 2012; Marchesi et al., 2011; Kostic et al., 2013).

When the differences were assessed at the level of OTUs, numerous changes between the normal and tumor microbiomes were observed with significant differences in the abundances of 19 different taxa (Wilcoxon signed rank test q≤0.1 after FDR correction). Of note, the tumors showed decreases in the abundances of several taxa within the order Chlostridales, namely, Lachnospiraceae, Ruminococcaceae, and Faecaibacterium prausnitzii, as well as several members within the order Bacteroidales, including Bacteroides, Rikenellaceae, and Bacteroides uniformis. Taxa that were enriched in the tumor microbiomes included Fusobacterium and several Proteobacteria genera, including Candidatus, Portiera and Providencia. Both Fusobacterium and Providencia are known pathogens, and when a correlation network is generated, it is clear that there are correlated abundance changes in the microbiome as a function of their presence. For instance, Fusobacterium species have been shown to have a mutualistic relationship with some Pseudomonas species at abscesses (Brook et al., 1986). This co-occurrence is seen in the data as a positive correlation between the abundances of the two genera. Other specific interactions between different bacterial taxa remain speculative. In the case of Lactobacillus in the human microbiome, it has been demonstrated that there can be reciprocal interference between species in this genus and other bacterial species in the form of competition for epithelial cell adhesion. As both Lactobacillus and Providencia utilize cell adhesion in their colonization of the human body, this may explain the negative correlation between the two genera in the dataset. While there was not a significant correlation between the relative abundances of Fusobacterium and Providencia in this analysis, the overlap among patients who showed increased levels of these genera at the tumor sites was assessed. Taken individually, Fusobacterium and Providencia were more abundant in the tumor microenvironment of 23 out of 44 and 28 out of 44 patients, respectively. Nineteen out of 44 patients showed increases in both of the genera in their tumor microenvironments with respect to their normal matched tissue microbiomes.

### CRC-Associated Microbiome Diversity

The alpha-diversity was calculated using a variety of metrics within each of the samples using QIIME (Caporaso et al., 2010). Alpha diversity metrics that account for phylogenetic relationships between the OTUs show that the tumor microbiomes exhibited higher alpha diversity than those of the normal, patient-matched microbiomes (p=0.029 by two-sided Wilcoxon signed rank test). This is also true when using alternative measures of diversity such as the Shannon's index or the Inverse Simpson's (p=0.020 and 0.024, respectively, by Wilcoxon signed rank test).

### Variation in the Functional Pathways and Enzymes in the Tumor Microbiome

A recent report presented the validation of a pipeline that leverages knowledge of the human gut reference genomes to predict general microbiome function and enzyme composition from 16S rRNA gene sequencing data (Langille et al., 2013). While this approach is not suitable for making conclusive statements regarding single, specific enzymes, it is appropriate for general functional comparisons between groups of samples, as is the case in this report. Using this validated pipeline—Phylogenetic Investigation of Communities by Reconstruction of Unobserved States (PICRUSt)—a virtual metagenome was constructed for each of the samples' microbiomes (Langille et al., 2013). The KEGG database was used as a reference to determine the abundances of metabolic pathways and enzymes within the virtual metagenomes (Kanehisa et al., 2014; Kanehisa and Goto, 2000). As with the bacterial phyla, significant variation was seen in the predicted functional pathways represented within each of the sampled microbiomes, though, as expected from previous studies, the variability in phylum abundances is far greater than the variability in the functional pathways (The Human Microbiome Consortium, 2012; Carroll et al., 2010). It is important to note that the results of this analysis are predictions only and not direct measurements of sequences that correspond to pathway member or enzyme genes. Despite the validation of this prediction approach, it is possible that this method biases the predictions toward microbial genomes that are well documented to the exclusion of other, unknown or poorly documented taxa.

These observations highlight the substantial functional redundancy across the phyla. In other words, diversity among the taxa within a given microbiome can mask the functional similarities—taxonomically distinct microbes in the gut can operate identically, or nearly so, at the functional level. The patient-by-patient variability in phyla does not perfectly correspond to that seen at the functional pathway level, though, as expected, analysis at the level of enzymes and pathways provides insights that analyses of the taxa alone may not be able to identify, due to the high level of between-sample variation. In general, the differences seen at the pathway level are roughly an order of magnitude less than the differences seen at the phylum level. Although the pathway differences are smaller than those at the level of the phylum, there remain physiologically relevant, statistically significant changes between the normal and tumor metagenomes.

Twenty pathways (as defined by KEGG, level 3) were found to be differentially abundant between the tumor and normal tissue. Alanine, aspartate, and glutamate metabolism, DNA replication proteins, and starch and sucrose metabolism were significantly depleted in the tumor microbiome (q≤0.01 for each pathway by two-sided Wilcoxon signed rank test after FDR correction). Conversely, secretion system, two-component system, and bacterial motility protein pathways were significantly enriched in the tumor microbiome (q≤0.04 for each pathway by two-sided Wilcoxon signed rank test after FDR correction).

To more closely examine the variation in the microbiome virulence potential, information regarding virulence association from MVirDB was used to annotate the predicted enzymes (Zhou et al., 2007). The tumor-associated microbiome was found to be significantly enriched with enzymes related to microbial virulence. This enrichment was significant when including all possible virulence categories (p=0.0046 by Fisher's exact test). Additionally, when assessing enrichment for virulence-related genes by known functional categories in MVirDB, the tumors were significantly enriched for genes encoding general virulence proteins (p=5.8×10⁻⁵, by Fisher's exact test). Genes encoding bacterial toxins were also found at higher abundance in the tumor, but the enrichment was not statistically significant (p=0.17 by Fisher's exact test), likely due to low total gene counts for some categories (e.g., protein toxins).

This virtual enrichment was predicted to be driven by known pathogenic bacteria within the microbiome, i.e., Providencia and Fusobacterium. In fact, when a comparison is made among the virulence-associated genes significantly differentially found in the tumor microenvironment (155), the virulence genes associated with Providencia (333), and the virulence genes associated with Fusobacterium (209), there is substantial overlap among and between the three different groups. In order to exclude the possibility that the virulence enrichment and corresponding overlap between the virulence genes found at the tumor site were the result of non-specific effects rather than due to the potential contributions of Providencia and Fusobacterium, the entire PICRUSt pipeline and subsequent enrichment analyses were repeated using an OTU table from QIIME with both Fusobacterium and Providencia explicitly excluded. In this case, while there were still 123 virulence genes from other taxa associated with the tumor microbiome, the enrichment is not significant compared with the background of normal tissue from the same cancer patients (Fisher's exact test one-sided p value>0.9). This demonstrates that the virulence signature in the microenvironment is dependent on Fusobacterium and Providencia.

To highlight the differences seen when assessing patient-matched tissue samples compared with assessing case and control stool samples, we performed a comparison of our results with those of Zackular et al. (2014). Zackular et al. performed an assessment of the microbiomes of CRC patients' stool samples relative to those of normal patients or patients with colorectal adenomas (Zackular et al., 2014). In comparison with the data described in this example, a prediction would be that both Fusobacterium and Providencia would be found at increased abundances in CRC patients relative to normal controls. While the data from Zackular et al. show a statistically significant increase in Fusobacterium in CRC patients' stools, Providencia at the genus level was not detected in the stool samples. However, there was a clear trend showing a doubling of the abundance of Enterobacteriaceae (the family to which Providencia belongs) when looking at stools from normal patients in comparison with stools from patients with adenomas and yet again when comparing adenoma patients and CRC patients. A likely explanation for the difference between this example and Zackular et al. is that data in the latter study were collected from stools and were not patient-matched. Thus, inter-individual variability likely decreased the ability to identify some tumor-associated taxa, such as Providencia.

### DISCUSSION

At the phylum level, the differences seen between the normal and tumor tissue-associated microbiomes are consistent with many previous reports (Bonnet et al., 2014; Kostic at al., 2012; Ahn et al., 2013; Shen et al., 2010; Kostic et al., 2013; Arthur et al., 2012; Wu et al., 2013). When assessing the data using information that accounts for more fine-grained detail with respect to taxonomy, we have made several important findings were made. Two of the genera we found to be enriched in the tumor microbiome, Providencia and Fusobacteria, are known to be pathogenic; Fusobacteria has been implicated previously in CRC (Castellarin et al., 2012; Kostic at al., 2012; Marchesi et al., 2011; Kostic et al., 2013).

Species belonging to the genus Providencia have been implicated as infectious agents causing urinary tract infections, ocular infections, and gastroenteritis (Shima et al., 2012; Koreishi et al., 2006; Murata et al., 2001; Lau et al., 2004; Kholodkova et al., 1977; Asakura et al., 2013; Shima et al., 2012; Yoh et al., 2005). In addition, it is a genus in which several sub-strains have acquired resistance to commonly used antibiotics (Lau et al., 2004; Lee et al., 2007; Luzzaro et al., 2006). Fusobacterium is a genus that encompasses several species known to be pathogenic in humans; they are obligate anaerobes, with known sites of infection in the oral cavity as well as in the gastrointestinal tract (Kolenbrander et al., 2000; Allen-Vercoe et al., 2011). The finding that these particular genera are prevalent in the tumor microenvironment suggests several alternative, though not mutually exclusive, hypotheses. One possibility is that these bacteria are causative in oncogenesis or tumor progression; another possibility is that these species are being enriched as the tumor has formed a niche that favors these bacteria. In the case of Fusobacteria, the results from several different studies, both correlative and mechanistic, indicate that it is likely a cancer driver (Castellarin et al., 2012; Rubinstein et al., 2013; Kostic et al., 2013). In the case of Providencia, there are as yet no definitive studies that implicate this genus as a contributor to CRC. The discovery of Providencia in the tumor microbiome is interesting as, similar to Fusobacteria, it encodes a potent, immunogenic lipopolysaccharide (Asakura et al., 2012; Onoue et al., 1996). In fact, several virulence genes responsible for lipopolysaccharide biosynthesis are shared by both genera and are also significantly increased in the tumor microenvironment A recent study, using Drosophila as a model system, performed a genomic comparison of four different species of Providencia isolated from the human gut (Galac and Lazzaro, 2012). These researchers demonstrated that these four species share common sets of virulence-related genes, including a type 3 secretion system and genes for cell adhesion. Additionally, Providencia has been shown to disrupt the epithelial membrane in the intestines, though the mechanism by which this is accomplished is still unclear (Murata at al., 2001; Asakura et al., 2013; Albert et al., 1992; Guth and Perrella, 1996). These factors manifest phenotypically as gastroenteritis, though with our discovery of its association with the cancer microenvironment, it is a promising candidate cancer-promoting pathogen (Yoh et al., 2005).

From a diagnostic and therapeutic perspective, assessing the CRC-associated microbiome by testing for differentially abundant taxa is an eminently worthwhile endeavor as it is the logical location to look for specific taxa that could be biomarkers and/or targets for intervention in CRC. However, it is possible that the search for specific taxa might miss the larger perspective. For instance, as described above, Fusobacteria and Providencia share many important phenotypic characteristics—potent, immunogenic lipopolysaccharide and the ability to damage colorectal tissue. These similarities might be better assessed using metagenomic or metatranscriptomic approaches, virtual or otherwise, as these key features are undoubtedly reflected in the genes that these particular bacteria encode, many of which are shared virulence factors with known detrimental properties.

Defining a clear set of virulence factors a priori as targets of interest and assessing their relative expression is a promising approach to CRC therapy. This report proposes such an approach by showing the striking predicted enrichment of virulence genes in the tumor-associated microbiome, potentially driven by Fusobacteria and Providencia. The fact that virulence proteins are predicted to be enriched in the tumor-associated microbiome lends support to the hypothesis that the microbiome is an active contributor to CRC and not just a passive byproduct of the changes the tumor makes in the organ. In the case of F. nucleatum, it is clear that there is a direct functional link between the bacteria and cancer development, though more work using cell culture and model organisms will be needed in the future to empirically assess the mechanistic interplay between colorectal tissues and specific components of the microbiome (Rubinstein et al., 2013). It is important to note that this clear enrichment is likely underestimated because MVirDB, while expansive, does not currently encompass all known virulence genes in the microbiome, and, as the field of medical microbial genomics advances, new virulence genes will undoubtedly be discovered. For instance, the FadA protein from F. nucleatum has been reported as a critical virulence factor, yet as it is a recent report, this finding has not yet made its way into MVirDB as of this submission (Rubinstein et al., Zhou et al., 2007).

It is important to note that this research uses 16S rRNA gene sequences as the starting point. Although this approach has obvious benefits in terms of resource expenditures and computational processing, there are several potential disadvantages. First, the microbiome functional assessment presented here uses a prediction method that, while validated and robust when applied to human gut samples, remains a prediction and may not necessarily perfectly reflect the biological reality (Langille et al., 2013). Another concern, as with all DNA-based approaches, is that even when a gene is predicted in a sample, it may still not be expressed or active. Additional metatranscriptomic research will undoubtedly shed light on this situation in the future.

### CONCLUSIONS

It is clear that there are numerous taxa in the CRC microbiome that are correlated with the disease. Here, in addition to the previously reported genus Fusobacterium, we report the discovery of another genus with similar pathogenic features, Providencia. This manuscript also presents an analysis that incorporates predicted information at the functional (e.g., virulence potential) level to assess differences between the normal and cancer-associated microbiomes. It is important to note that these two approaches (taxonomy-based and function-based) provide different yet interdependent information about the microbes in the tissue microenvironment. Utilizing this combined approach, researchers can be provided with specific taxa as biomarkers and/or therapeutic targets while also looking globally at the predicted pathogenic potential of the microbiome and showing a clear predicted enrichment of virulence-associated microbial genes present in the CRC microbiome. As with the bacterial genera associated with the disease, these virulence genes may provide researchers and clinicians with targets for therapeutic intervention to improve patient outcomes.

The invention will be described by the following non-limiting examples.

### Example I

### Methods

Samples Used in this Analysis.

88 tissue samples from 44 individuals were used, with one tumor and one normal sample from each individual. These de-identified samples were obtained from the University of Minnesota Biological Materials Procurement Network (Bionet), a facility that archives research samples from patients who have provided written, informed consent. All research conformed to the Helsinki Declaration and was approved by the University of Minnesota Institutional Review Board, protocol 1310E44403. Tissue pairs were resected concurrently, rinsed with sterile water, flash frozen in liquid nitrogen, and characterized by staff pathologists. The criteria for selection were limited to the availability of patient-matched normal and tumor tissue specimens.

### Microbiome Data Generation.

Total DNA was isolated from the flash-frozen tissue samples and their associated microbiomes by adapting an established nucleic acid extraction protocol (Burns et al., 2013). Briefly, approximately 100 mg of flash-frozen tissue were physically disrupted by placing the tissue in 1 mL of Qiazol lysis solution and sonicating in a heated (65° C.) ultrasonic water bath for 1-2 hours. The efficiency of this approach was verified by observing high abundances of Gram-negative bacteria across all samples, including those from the phylum Firmicutes. Additionally, sequences from the notoriously difficult to lyse bacterial genera Mycobacterium and/or Bacillus (Vandeventer et al., 2011) were detected in the majority of samples, also indicating a rigorous and efficient lysis. DNA was purified from the lysate using the Qiagen All-prep kit (Qiagen Inc., Valencia, Calif.).

In preparation for 16S rRNA gene sequencing, DNA isolated from colon samples was quantified by quantitative PCR (qPCR), and the V5-V6 regions of the 16S rRNA gene were PCR amplified with the addition of barcodes for multiplexing. The forward and reverse primers were the V5F and V6R sets from Ca, et al. (2013), the disclosure of which is incorporated by reference herein. The PCR conditions were as follows. Amplification was carried out in a 25 μL PCR reaction with 5 μL of template DNA with an initial denaturation step at 95° C. for 5 minutes followed by 30 cycles of denaturation (50 seconds at 94° C.), annealing (30 seconds at 40° C.), and elongation (30 seconds at 72° C.). Amplified samples were then diluted 1:100 in water for input into library tailing PCR. This PCR reaction was similar to initial amplification except the PCR conditions consisted of an initial denaturation at 95° C. for 5 minutes followed by 15 cycles of denaturation (50 seconds at 94° C.), annealing (30 seconds at 40° C.), and elongation (1 minute at 72° C.). Quantification of PCR products was performed using the Quant-iT PicoGreen dsDNA Assay Kit (Life Technologies Grand Island, N.Y.). A subset of the amplified libraries was spot-checked on a Bioanalyzer High-Sensitivity DNA Chip (Agilent Technologies, Santa Clara, Calif.) to ascertain if the amplicons were the predicted size. These samples were each normalized to 2 nM and pooled. The total volume of the libraries was reduced using a SpeedVac and amplicons were size-selected at 420 bp±20% using the Caliper XT (Perkin Elmer, Waltham, Mass.). The pooled libraries were cleaned with 1.8×AMPureXP beads (Beckman Coulter, Brea, Calif.) and eluted with water. DNA concentration in the final pool was assayed with PicoGreen and normalized to 2 nM for input into Illumina MiSeq (v3 Kit) to produce 2×250 bp sequencing products. Clustering was performed at 10 pM with a 5% spike of PhiX. A single lane on an Illumina MiSeq instrument was used to generate the 16S rRNA gene sequences.

### Microbiome Data Analysis.

The sequence data contained approximately 21.4 million reads passing quality filtering in total, inclusive of forward and reverse reads, with a mean value of 242,940 quality reads per sample. The forward and reverse read pairs were merged using the USEARCH v7 program ‘fast_mergepairs’, allowing stagger, with no mismatches allowed (Edgar 2010). Merged reads were quality trimmed, again using USEARCH, to truncate reads at any quality scores of 20 or less. Following merging and trimming, there were an average of 62,100 high quality reads per sample (median 48.817; range 6559-173,471). The fasta sequence headers were renamed using a custom script to conform to QIIME standards.

The merged and filtered reads were used to pick operational taxonomic units (OTUs) with QIIME v1.7.0 using ‘pick_otus.py’, with the closed-reference usesearch_ref OTU picking protocol against the Greengenes database (August 2013 release) at 97% similarity (Caporaso et al. 2010; Navas-Molina et al. 2013; DeSantis et al. 2006). Reverse read matching was enabled, while reference-based chimera calling was disabled. Rarefaction was performed on the OTU table at 5000 reads prior to subsequent analyses.

### Exome Sequence Data Generation.

Genomic DNA samples were quantified using a fluorometric assay, the Quant-iT PicoGreen dsDNA Assay Kit (Life Technologies, Grand Island, N.Y.). Samples were considered passing quality control (QC) if they contained greater than 300 nanograms (ng) of DNA and display an A260:280 ratio above 1.7. Full workflow details for library preparation are outlined in the Nextera Rapid Capture Enrichment Protocol Guide (Illumina, Inc., San Diego, Calif.). In brief, libraries for Illumina next-generation sequencing were generated using Nextera library creation reagents (Illumina, Inc., San Diego, Calif.). A total of 50 ng of genomic DNA per sample were used as input for the library preparation. The DNA was tagmented (simultaneously tagged and fragmented) using Nextera transposome based fragmentation and transposition as part of the Nextera Rapid Capture Enrichment kit (Illumina. Inc., San Diego, Calif.). This process added Nextera adapters with complementarity to PCR primers containing sequences that allow addition of Illumina flow cell adapters and dual-indexed barcodes. The tagmented DNA was amplified using dual indexed barcoded primers. The amplified and indexed samples were pooled (8 samples per pool) and quantified to ensure appropriate DNA concentrations and fragment sizes using the fluorometric PicoGreen assay and the Bioanalyzer High-Sensitivity DNA Chip (Agilent Technologies, Santa Clara, Calif.). Libraries were considered to pass QC as long as they contained more than 500 ng of DNA and had an average peak size between 200-1000 base pairs. For hybridization and sequence capture, 500 nanograms of amplified library was hybridized to biotinylated oligonucleotide probes complementary to regions of interest at 58° C. for 24 hours. Library-probe hybrids were captured using streptavidin-coated magnetic beads and subjected to multiple washing steps to remove non-specifically bound material. The washed and eluted library was subjected to a second hybridization and capture to further enrich target sequences. The captured material was then amplified using 12 cycles of PCR. The captured, amplified libraries underwent QC using a Bioanalyzer, and fluorometric PicoGreen assay. Libraries were considered to pass QC as long as they contained a DNA concentration greater than 10 nM and had an average size between 300-400 base pairs. Libraries were hybridized to a paired end flow cell at a concentration of 10 pM and individual fragments were clonally amplified by bridge amplification on the Illumina cBot (Illumina, Inc., San Diego, Calif.). Eleven lanes on an Illumina HiSeq 2000 (Illumina, Inc., San Diego, Calif.) were required to generate the desired sequences. Once clustering was complete, the flow cell was loaded on the HiSeq 2000 and sequenced using Illumina's SBS chemistry at 100 bp per read. Upon completion of read 1, base pair index reads were performed to uniquely identify clustered libraries. Finally, the library fragments were resynthesized in the reverse direction and sequenced from the opposite end of the read 1 fragment thus producing the paired end read 2. Full workflow details are outlined in Illumina's cBot User Guide and HiSeq 2000 User Guides. Base call (.bcl) files for each cycle of sequencing were generated by Illumina Real Time Analysis (RTA) software. The base call files and run folders were then exported to servers maintained at the Minnesota Supercomputing Institute. Primary analysis and de-multiplexing was performed using Illumina's CASAVA software 1.8.2. The end result of the CASAVA workflow was de-multiplexed FASTQ files that were utilized in subsequent analysis for read QC, mapping, and mutation calling.

### Exome Data Analysis.

The exome sequence data contained approximately 4.2 billion reads in total following adapter removal and quality filtering, inclusive of forward and reverse reads, with a mean value of 47.8 million high-quality reads per sample. The raw reads were assessed using FastQC v0.11.2 and the Nextera adapters removed using cutadapt v1.8.1 (Anon n.d.; Martin, 2011). Simultaneously, cutadapt was used to trim reads at bases with quality scores less than 20. Reads shorter than 40 bases were excluded. The trimmed and filtered read pairs were aligned and mapped to the human reference genome (hg19) using bwa v0.7.10 resulting in a barn file for each patient sample (Li & Durbin, 2009). These files were further processed to sort the reads, add read groups, correct the mate-pair information, and mark and remove PCR duplicates using picard tools v1.133 and samtools v0.1.18 (Anon; Li et al. 2009). Tumor-specific mutations were identified using FreeBayes v0.9.14-24-gc292036 (Garrison & Marth, 2012). Following these steps, 94.0% of the remaining read pairs mapped to the reference genome, hg19. Specifically, SNPs only were assessed and a minimum coverage at each identified mutation position of more than 30× was required in both the patient normal and tumor samples. These mutations were filtered to only include those that were within protein-coding regions and compiled into a single vcf file. This vcf file was assessed using SNPeffect v4.1 K (2015-09-0) in order to predict the potential impact of each of the mutations (Cingolani et al., 2012). Based on these results, the mutations were grouped into three categories: (1) total mutations (2) non-synonymous mutations and (3) loss of function (LoF) mutations. The total mutations group is self-explanatory. The non-synonymous mutations included all the mutations in the total mutations group that were non-silent. The LoF group only included those mutations that resulted in a premature stop codon, a loss of a stop codon, or a frameshift mutation.

### Joint Analysis of Microbiome and Exome Data.

It was determined if there were taxa that differentiated patients with or without LoF mutation using LefSe (Segata et al. 2011). An LDA score (log 10)>2 was considered significant. Therefore, all the taxa with a LDA score (log 10)>2 were included in the calculation of the risk index. Then a risk index was built that is able to predict the presence or absence of a LoF mutation based on the OTU table collapsed at genus level. To build the risk index to predict the presence or absence of LoF mutation based on the taxonomy, the relative abundances (arcsine square root transformed) of the taxa associated with the LoF mutation (based on the LefSe output) were summed and the relative abundances of the taxa associated with no mutation (based on the LefSe output) were summed. Then the difference between these two sums (relative abundance of the taxa associated with absence of a LoF mutation minus relative abundance of the taxa associated with presence of a LoF mutation) was calculated, thereby obtaining a risk index. This procedure was repeated 44 times to obtain a risk index for each patient.

A leave-one-out procedure was also conducted to evaluate the taxa that differentiated patients with or without LoF mutation in the held-out patient, based on the LefSe output of n−1 patients. In detail, the taxa that differentiated patients with or without LoF mutation were identified using LefSe in the n−1 dataset. Then, the relative abundances of the taxa associated with the LoF mutation (based on the LefSe output of the n−1 dataset) were identified and the relative abundances of the taxa associated with no mutation (based on the LefSe output of the n−1 dataset) on the held-out patient summed. Then the difference between these two sums (relative abundance of the taxa associated with absence of a LoF mutation minus relative abundance of the taxa associated with presence of a LoF mutation) was calculated and a risk index obtained for the held-out patient. The procedure was repeated 44 times to produce a risk index in each of the held-out patients based on the difference between the sum of the taxa associated with the absence of LoF mutation minus the sum of the taxa associated with the presence of the LoF mutation found in each of the n−1 datasets. The significance of the difference in risk indexes between the patients with LoF mutation and patients with LoF mutation for each gene was assessed using a Mann-Whitney U test and a permutation test, in which we permuted the labels for a given gene 999 times, each time deriving new held-out predictions of the risk indexes for each subject for that gene. Then the observed difference in means between the patients with LoF mutation and patients with LoF mutation risk index predictions using the method on the actual LoF mutation labels to the differences observed in the permutations to obtain an empirical P-value was compared. This test was completed for 12 genes total, selected for the prevalence of LoF mutations in them in the patient population, and then the P-values corrected using the false discovery rate (FDR) correction for multiple hypothesis tests.

Receiving Operating Characteristic (ROC) curves were plotted and the area under the curve (AUC) values computed on a dataset containing 10 sets of predictions and corresponding labels obtained from 10-fold cross-validation using ROCR package in R (Sing et al., 2005). A risk index threshold was also obtained that best predicts the presence or absence of LoF mutation with a leave-one-out cross-validation on the risk index. Each held-out sample was treated as a new patient on whom the optimal risk index cutoff was tested and subsequently refined to separate patients who had a LoF mutation and patient who did not have a LoF.

### Results

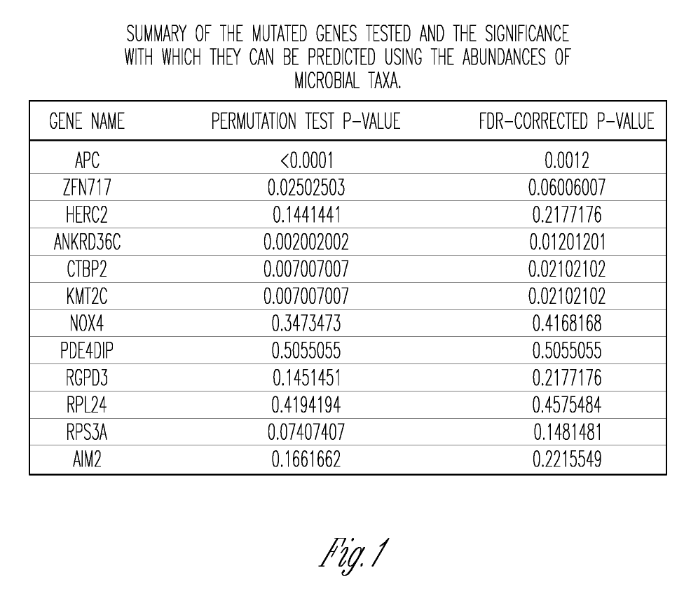

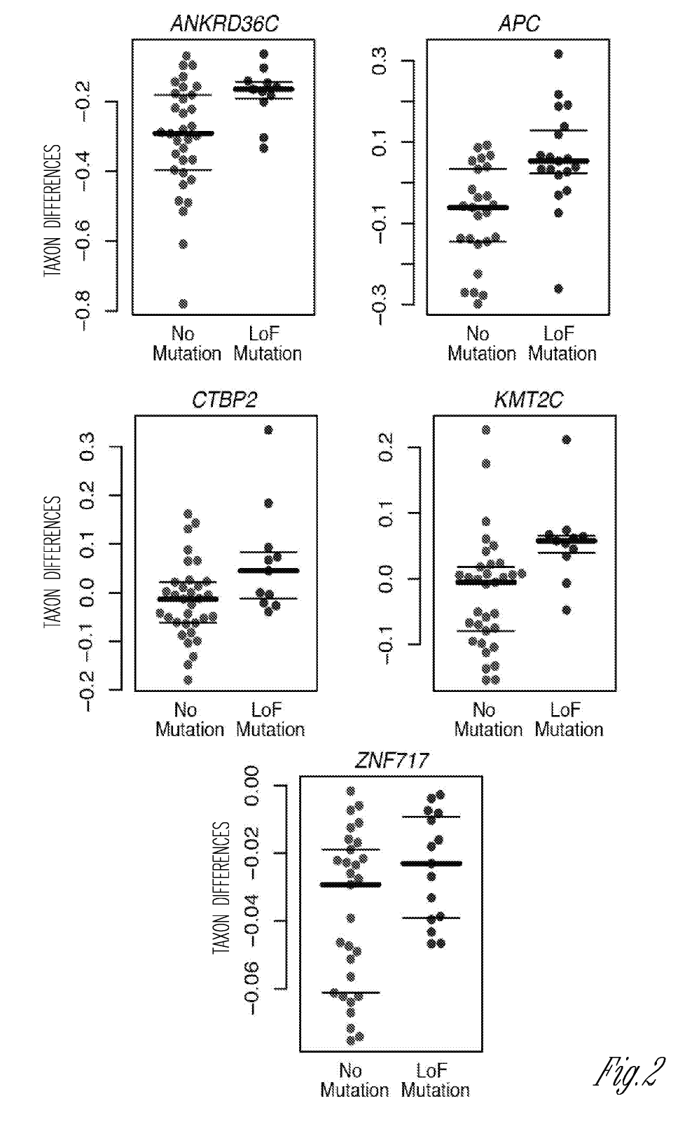

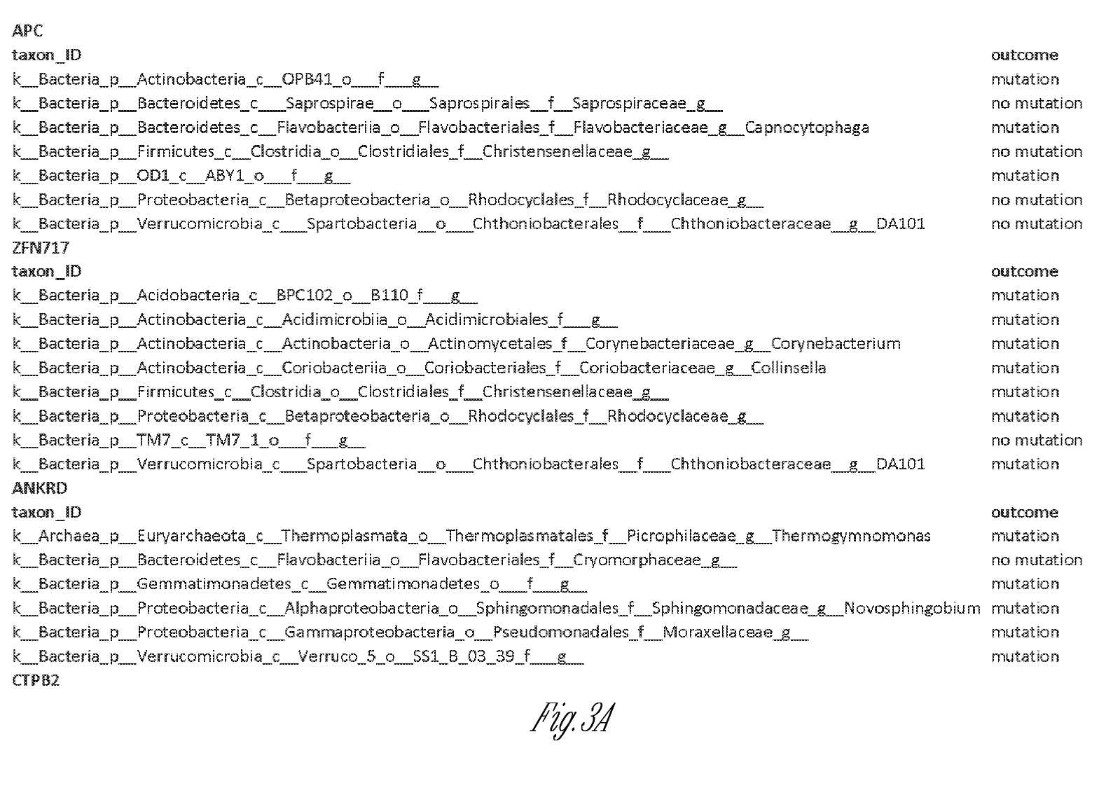

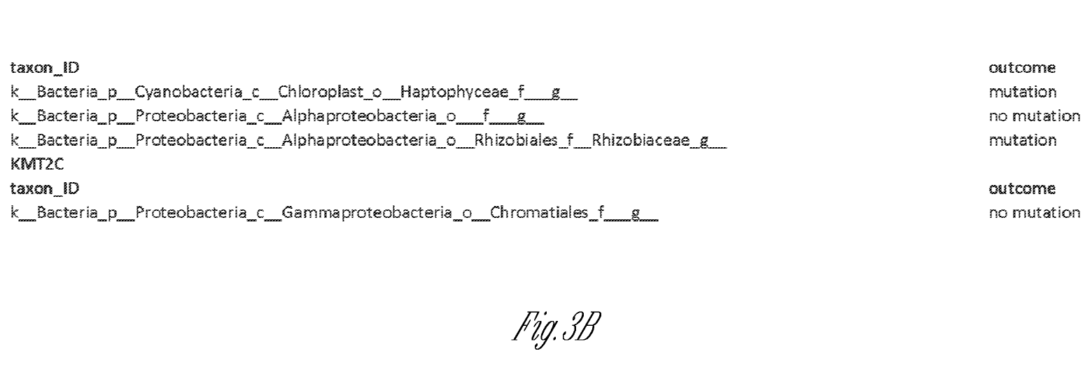

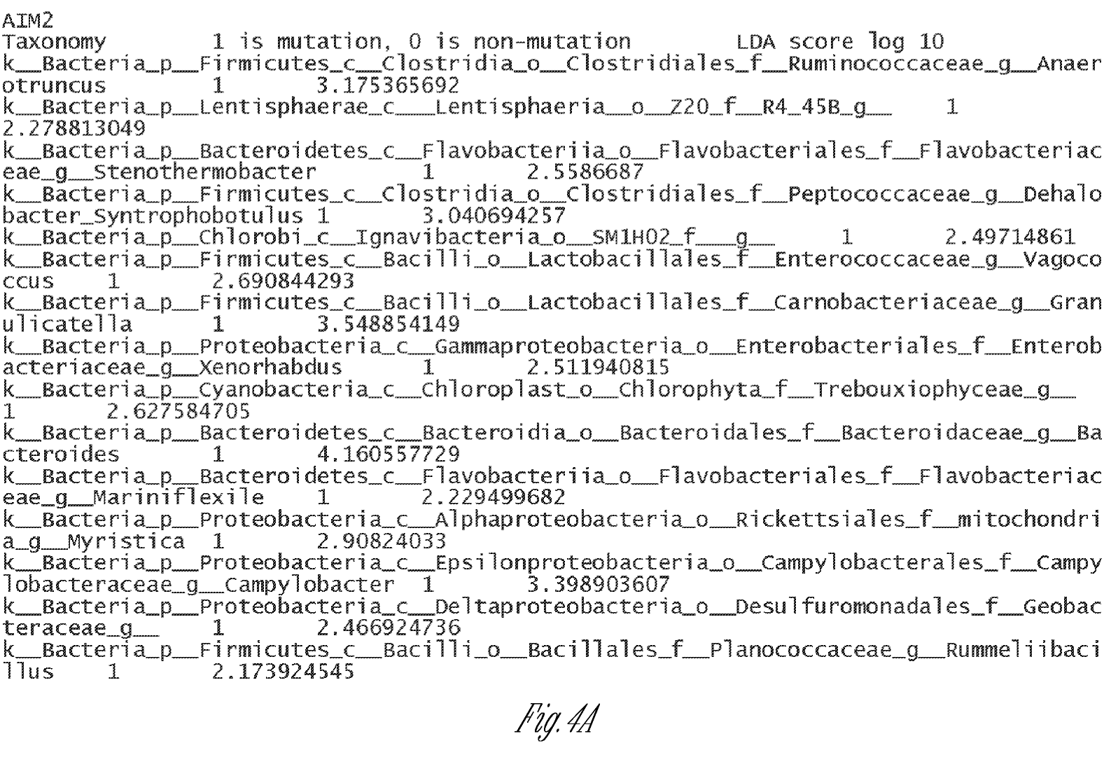

The results arrived at from the analyses above indicate that the presence of five different mutations is predicted based on the signals detected in the microbiome. Specifically, five of the genes indicated in FIG. 1 are able to be correctly predicted in this dataset with more than 70% sensitivity and 80% specificity.

### SUMMARY

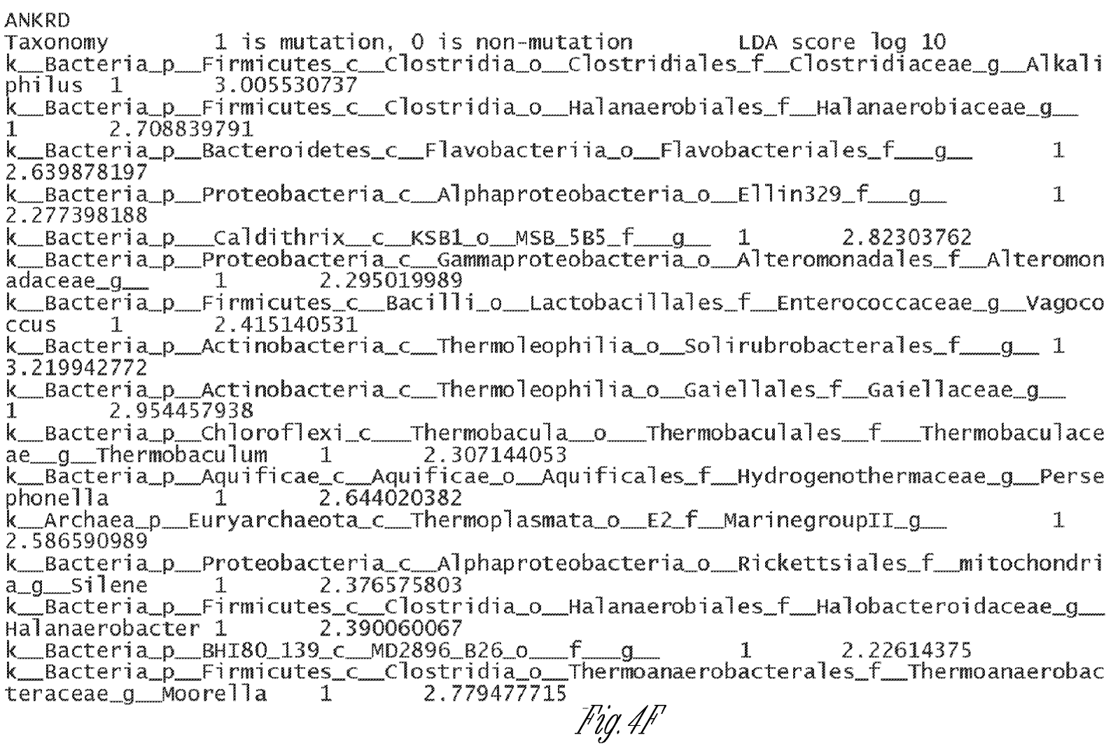

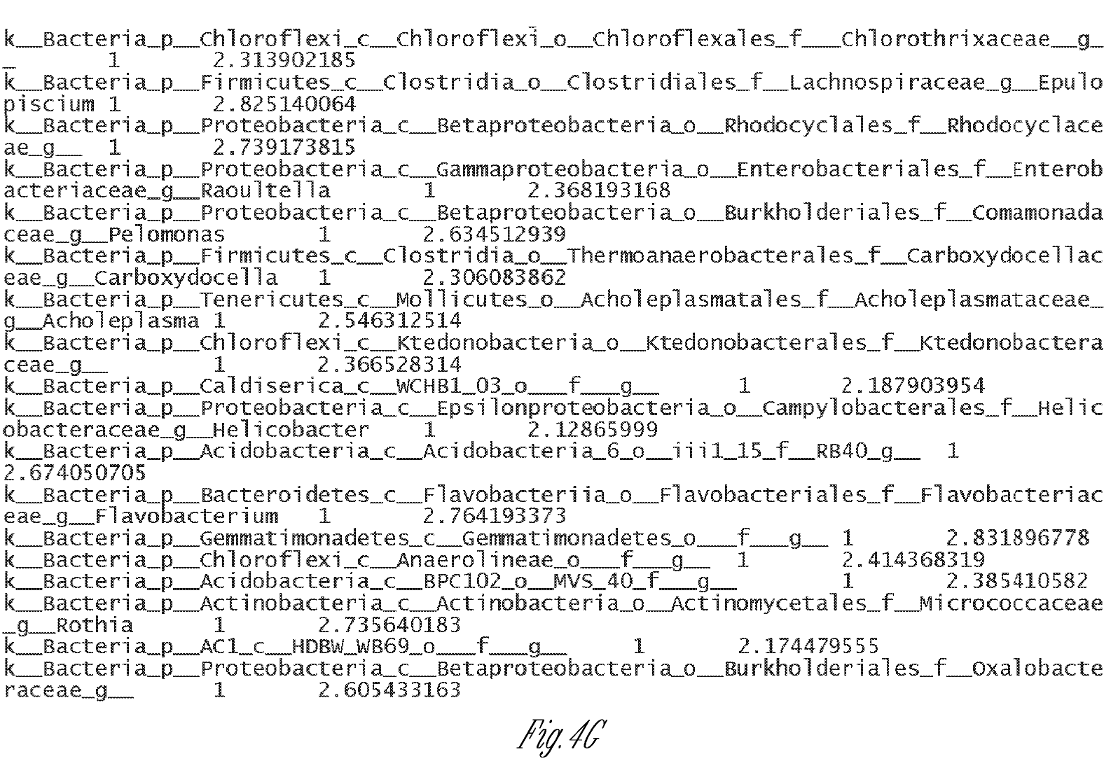

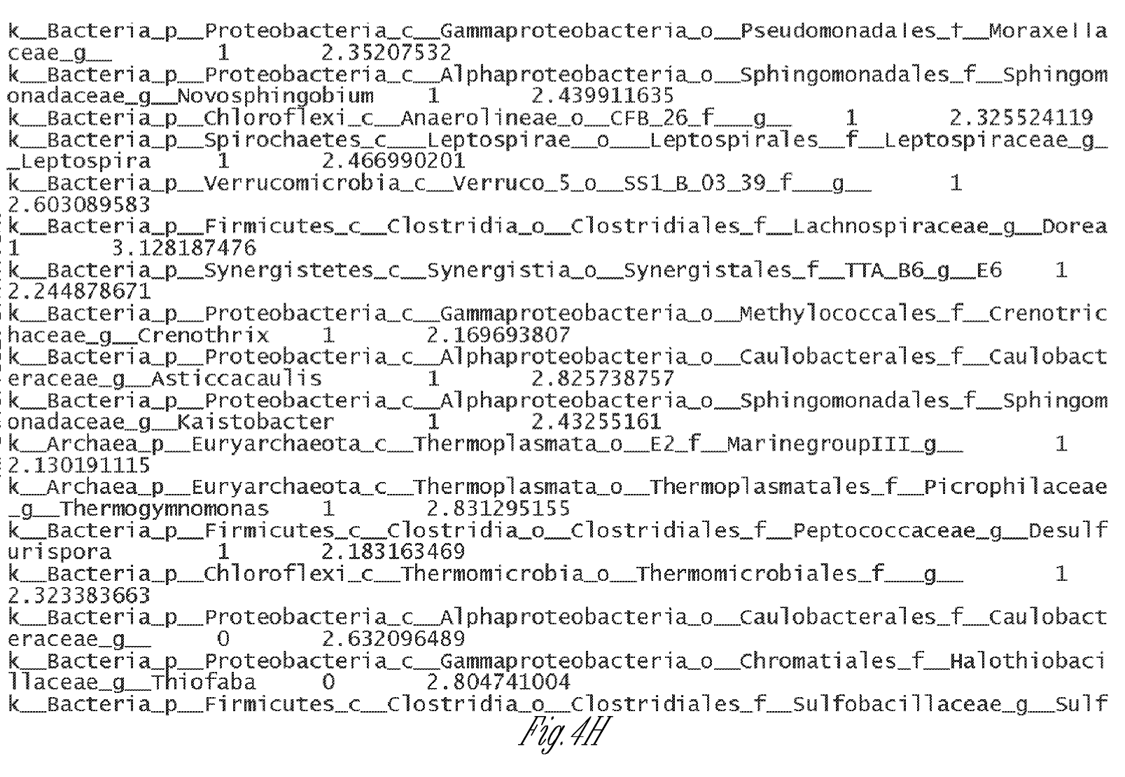

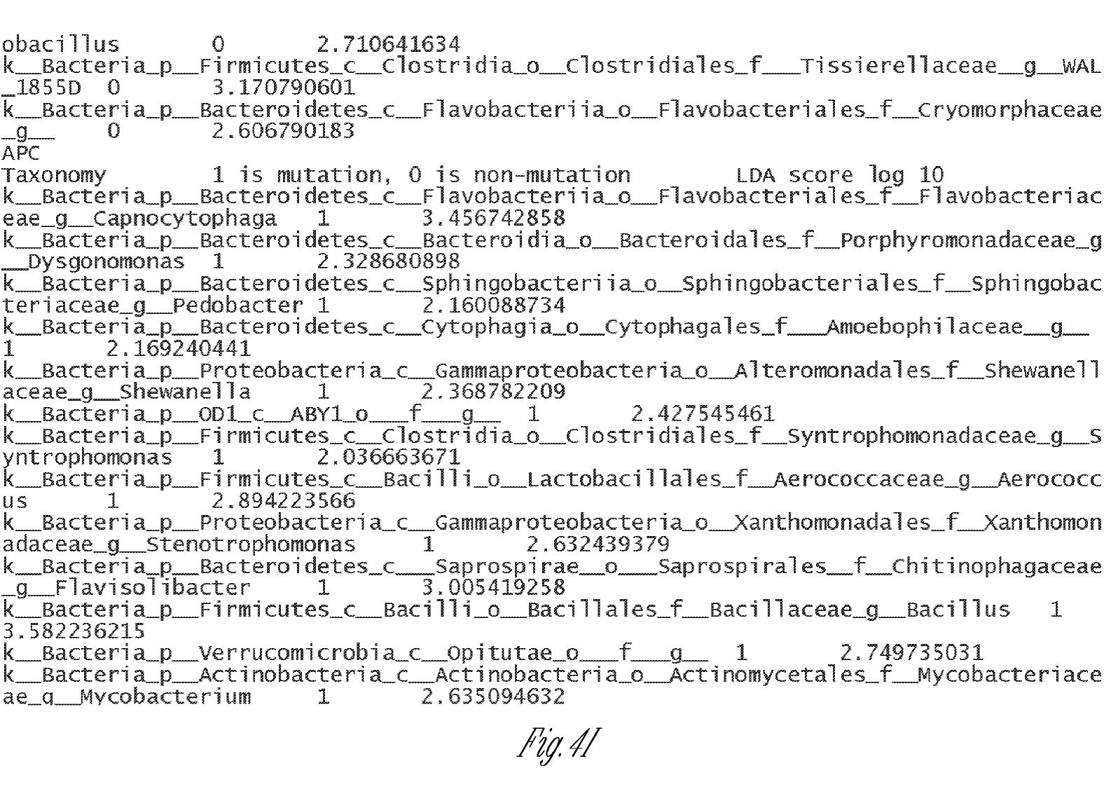

In summary, standard methods for bacterial 16S ribosomal RNA gene sequencing were used to determine the taxonomic profile of the bacterial community living in the tumor microenvironment. This type of analysis led to an association of specific bacterial taxa at the family, genus, and/or species level, with the presence of LoF mutations in particular tumor genes. From a linear combination of the relative abundances of these associated taxa, a LoF risk score was derived that was correlated with the presence of the LoF mutations. FIG. 3 provides bacterial species associated with LoF mutations for 5 different genes. Thus, this data provides for a predictive model in a stool test for categorization of tumor mutations in a non-invasive manner.

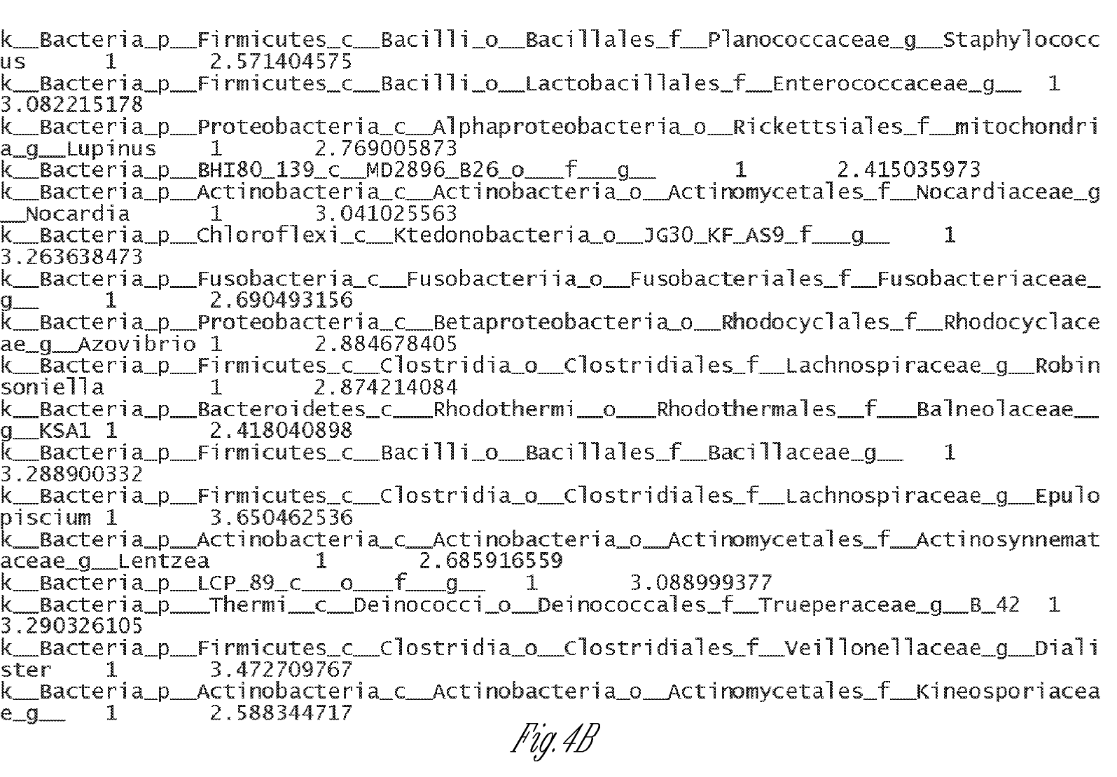

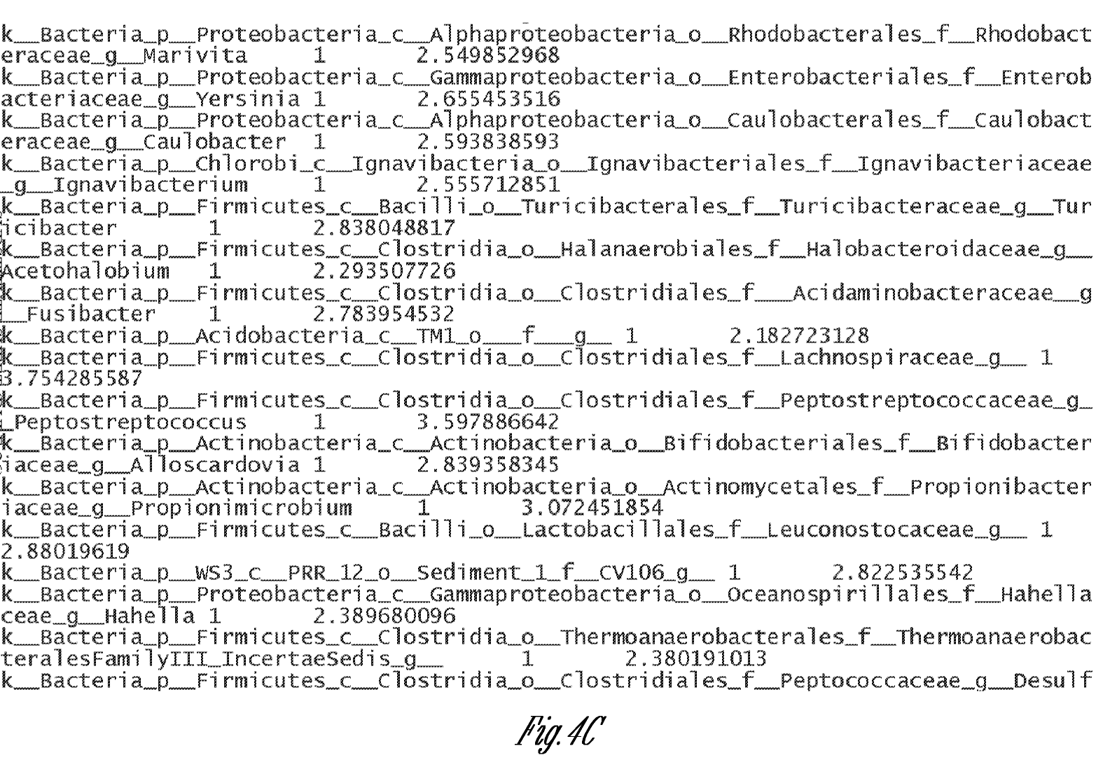

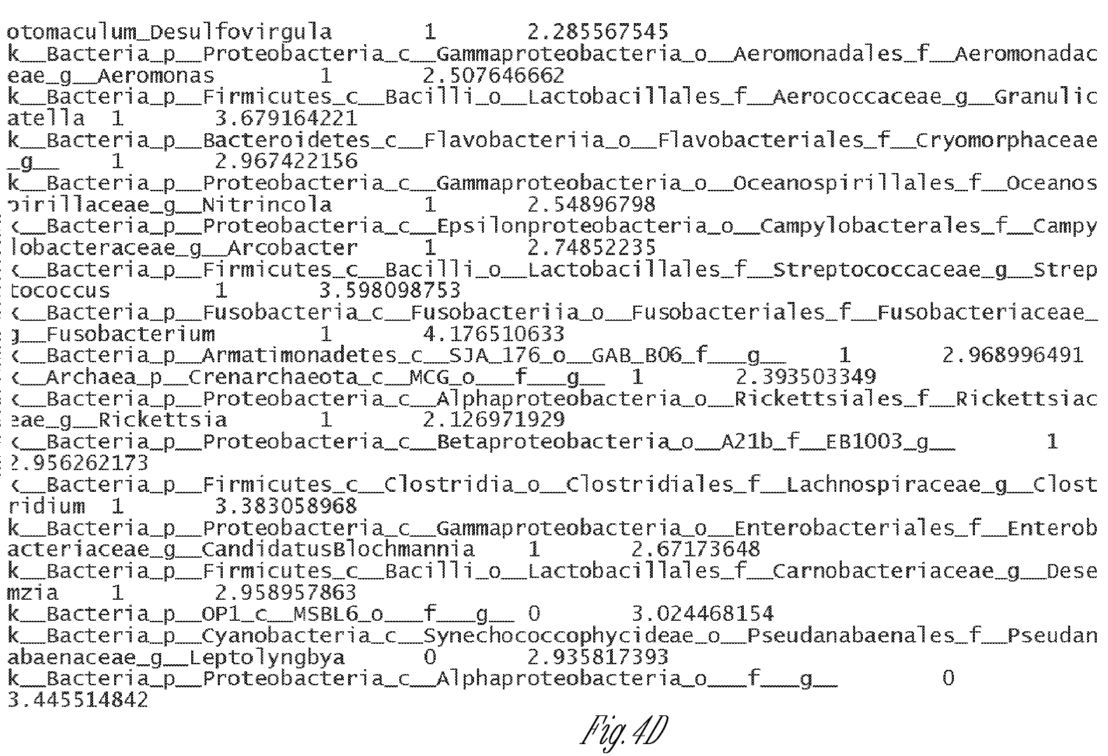

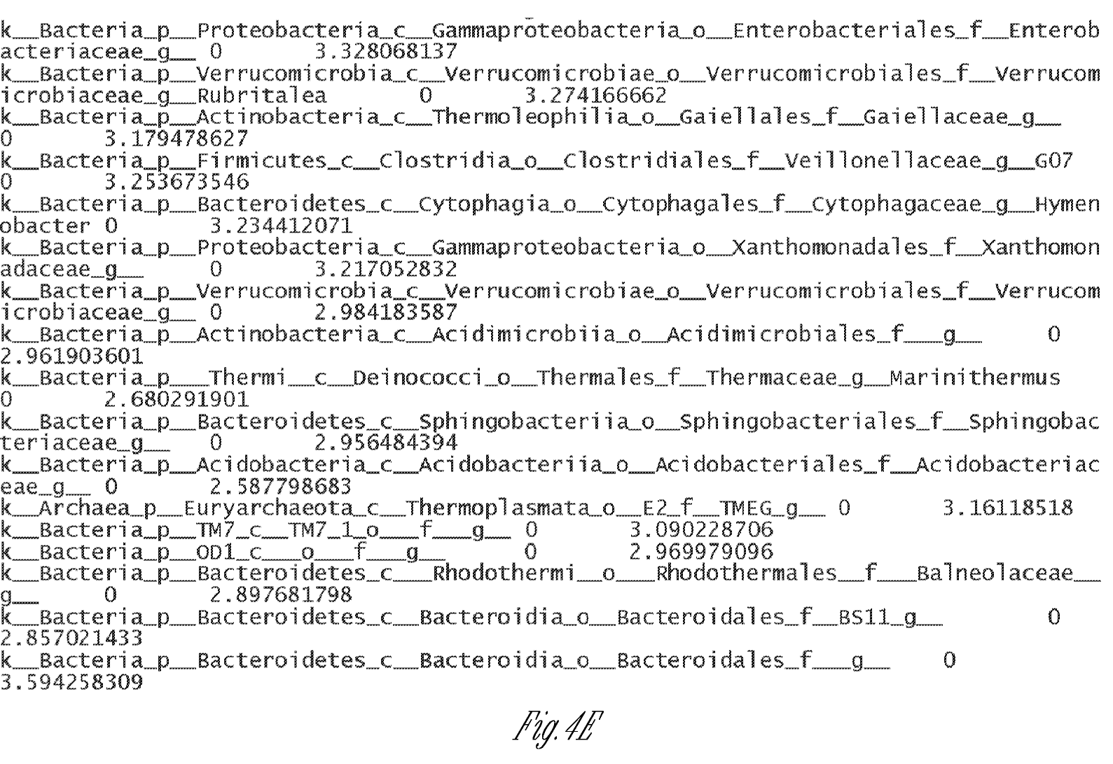

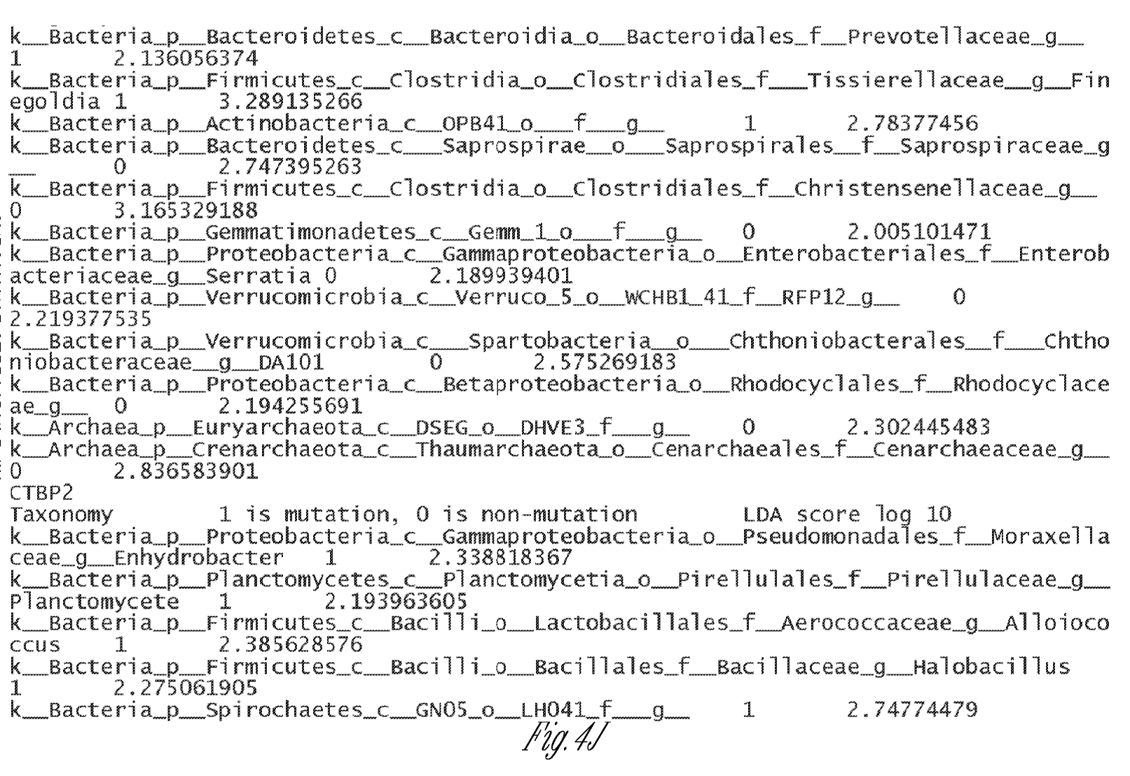

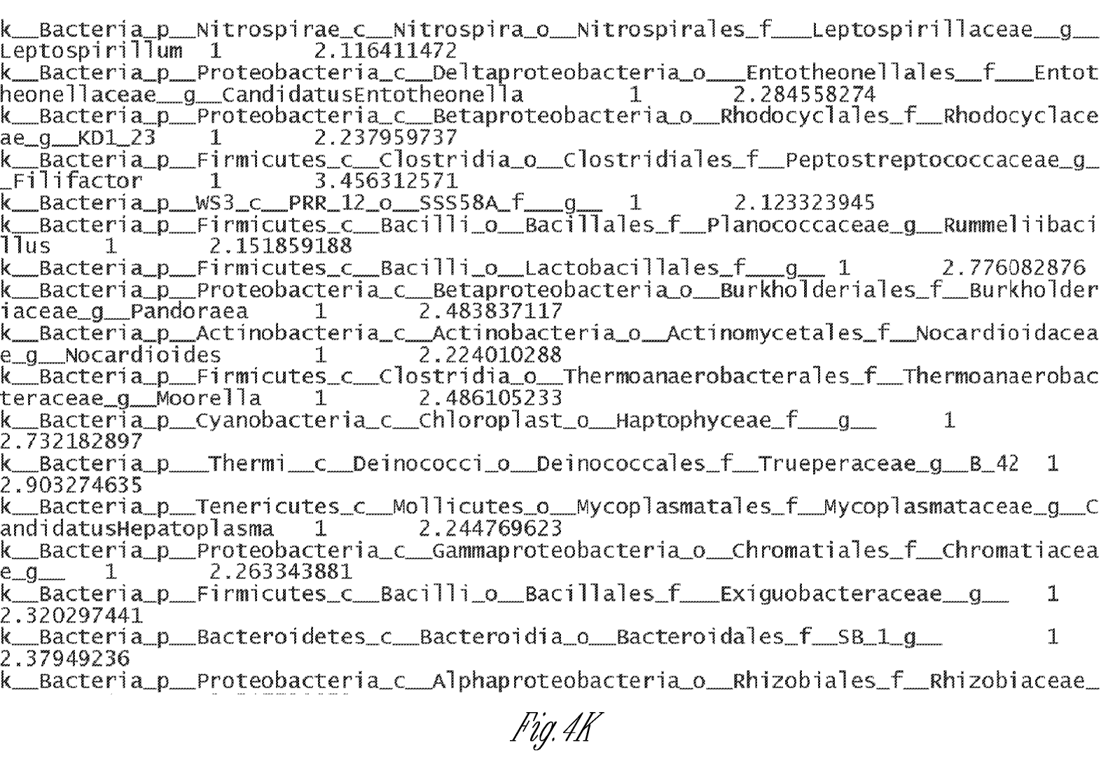

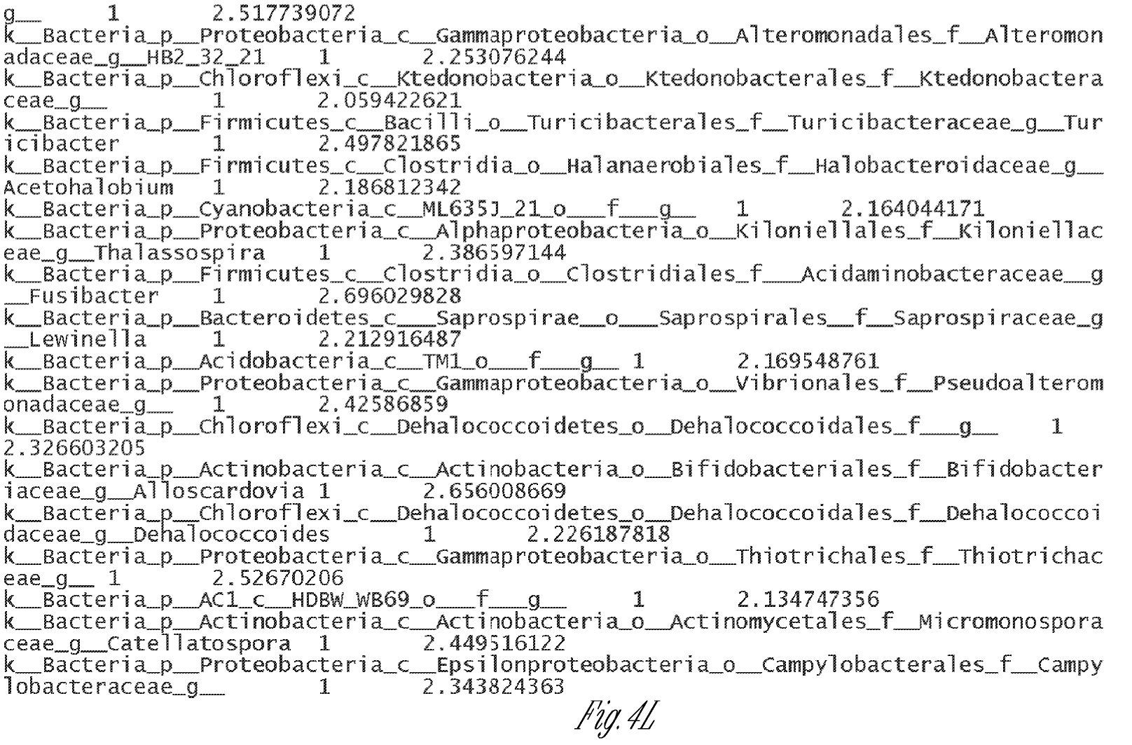

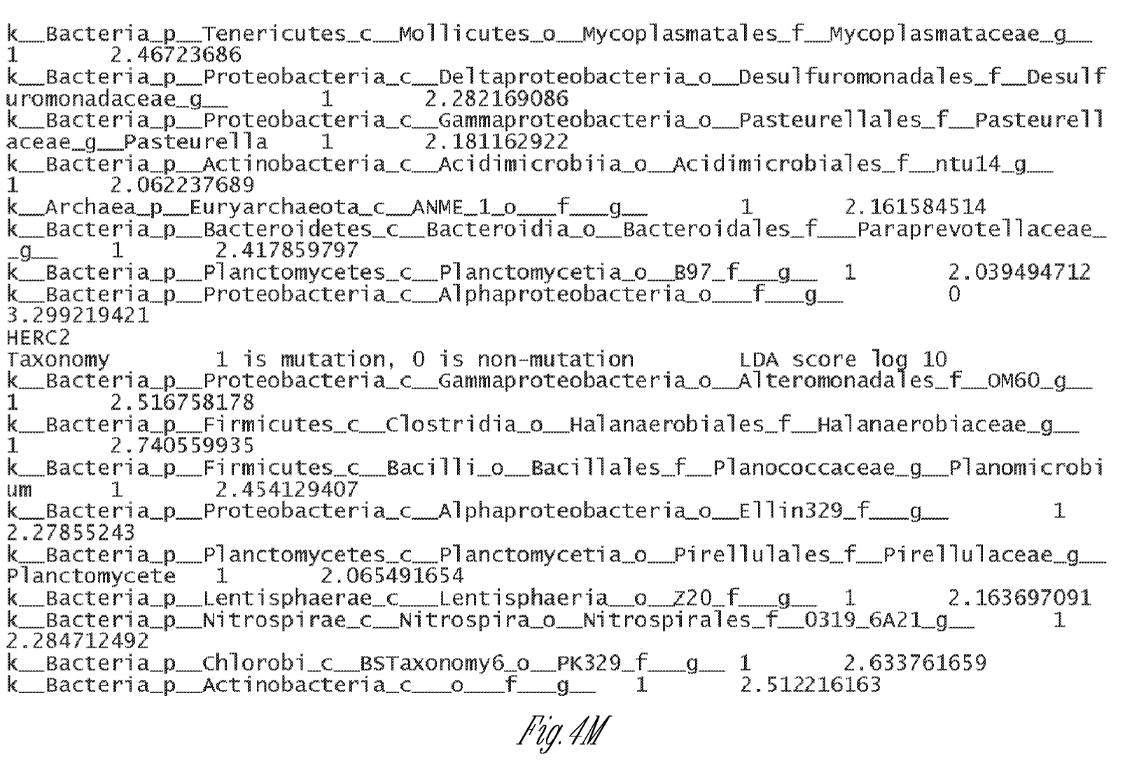

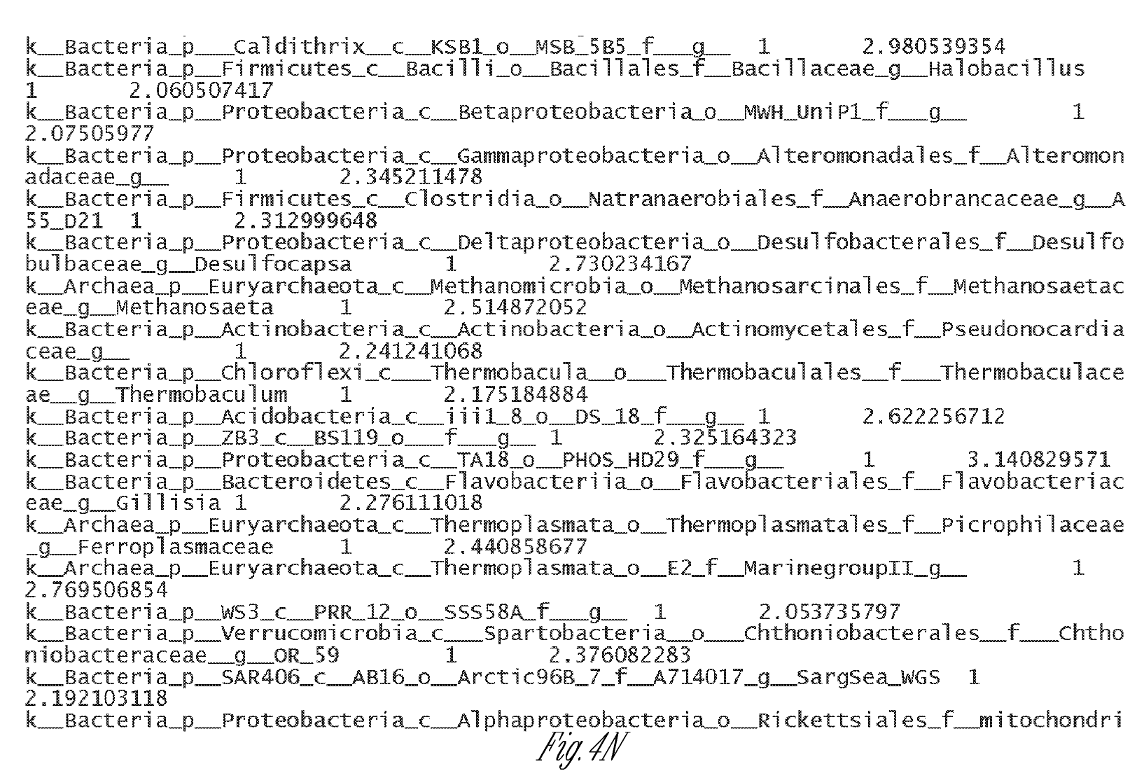

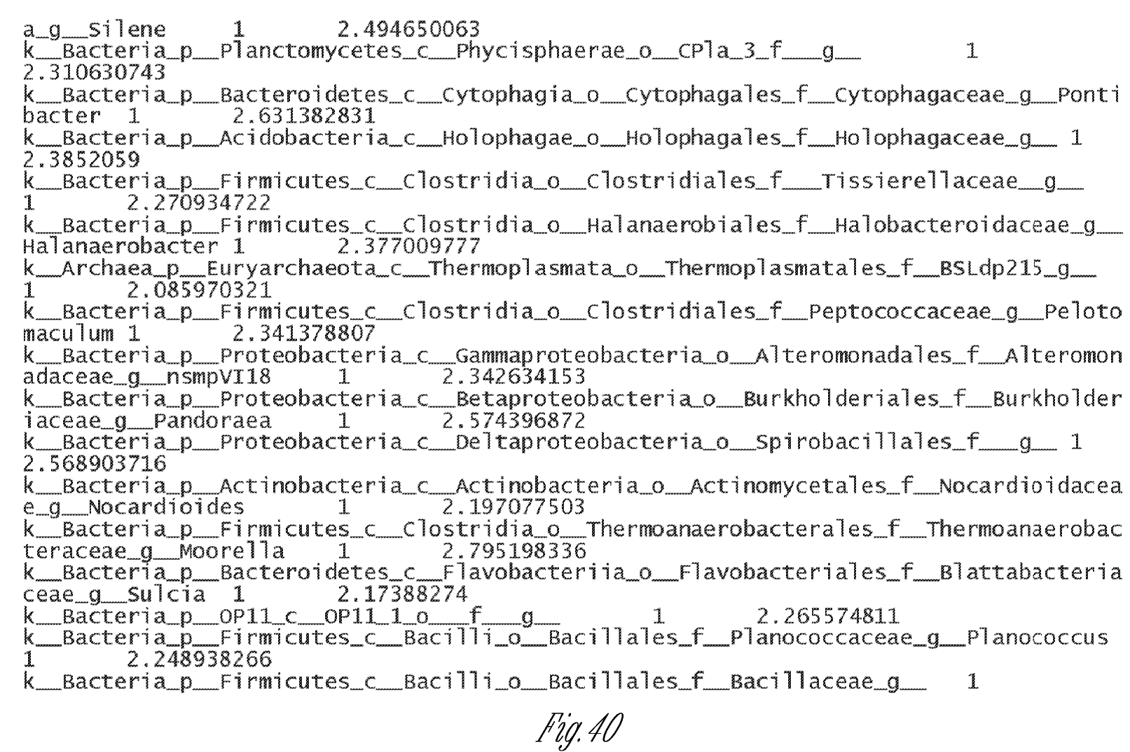

The risk score functions were derived from 44 tumor microenvironment tissue samples where the whole-exome sequence of the tumor genes was obtained as well as amplicon sequencing of the bacterial marker genes. Hold-out predictions of the risk indices for each subject, wherein each subject's index was predicted using a model trained only on the other subjects, are shown in FIG. 2. The significance of the difference in risk indices between the cases and controls (as determined by presence of LoF mutations) for each gene was assessed using a permutation test, in which the case/control labels for a given gene were permuted 10,000 times, each time deriving new hold-out predictions of the risk indices for each subject for that gene. Then the observed difference in means was compared between case and control risk index predictions using the actual LoF mutation labels to the differences observed in the permutations to obtain an empirical p-value. This test was performed in 12 genes total (see FIG. 4, which shows the taxa associated with each gene), the prevalence of LOF mutations in them was selected in the patient population, and the p-values corrected using the false discovery rate (FDR) correction for multiple hypothesis tests.

Although the contribution of bacterial species from the actual tumor micro environment is likely to be reduced in the bulk stool sample, making these species difficult to detect, a targeted assay for specific bacterial species may be employed.

### Example II

Variation in the gut microbiome has been linked to colorectal cancer (CRC), as well as to host genetics. However, it was unknown whether genetic mutations in CRC tumors interact with the structure and composition of the microbial communities surrounding the tumors, and if so, whether changes in the microbiome can be used as a predictor for tumor mutational status. Herein, the association between CRC tumor mutational landscape and its proximal microbial communities was investigated by performing whole-exome sequencing and microbiome profiling in tumors and normal colorectal tissue samples from the same patient. There was a significant association between loss-of-function mutations in relevant tumor genes and pathways and shifts in the abundances of specific sets of bacterial taxa. In addition, by constructing a risk index classifier from these sets of microbes, the existence of loss-of-function mutations in cancer-related genes and pathways, including MAPK and Wnt signaling, could be accurately predicted solely based on the composition of the microbiota. These results can serve as a starting point for understanding the interactions between host genetic alterations and proximal microbial communities in CRC, as well as for the development of individualized microbiota-targeted therapies.

### Introduction

The human gut is host to approximately a thousand different microbial species consisting of both commensal and potentially pathogenic members (Human Microbiome Project Consortium, 2012). In the context of colorectal cancer (CRC), it is clear that bacteria in the microbiome play a role in human cell signaling (Burns et al., 2015; Warren et al., 2013; Nakatsu et al., 2015; Wang et al., 2012; Shen et al., 2010; Chen et al., 2012; Zhu et al., 2014; Sing et al., 2014; Flemer et al., 2016; Wynedaele et al., 2015); for example, in the case of CRC tumors that are host to the bacterium Fusobacterium nucleatum, the microbial genome encodes a virulence factor, FadA, that can activate the β-catenin pathway (Rubinstein et al., 2013). In addition, several attempts have been made to predict CRC status using the microbiome as a biomarker (Zeller et al., 2014; Zackular et al., 2014; Yu et al., 2015; Baxter et al., 2016). It has been shown that focusing on a single bacterial species, F. nucleatum, it is possible to predict some clinically relevant features of the tumor present (Miora at al., 2014). However, as only a minority of CRCs are host to F. nucleatum, this is a somewhat limited application (Kostic et al., 2012). Additionally, in healthy individuals, host genetic variation can affect the composition of the microbiome (Goodrich et al., 2016; Blehkman et al., 2015; Goodrich et al., 2014; Knights et al., 2014; Davenport et al., 2015; Goodrich et al., 2016), and the associated human genetic variants are enriched in cancer-related genes and pathways (Blekhman et al., 2015). However, it is still unknown whether somatic mutations in host cells can affect the composition of the microbiome that directly interacts with host tissues.

### Methods

### Patient Inclusion and DNA Extraction

88 tissue samples from 44 individuals were used, with one tumor and one normal sample from each individual. These de-identified samples were obtained from the University of Minnesota Biological Materials Procurement Network (Bionet), a facility that archives research samples from patients who have provided written, informed consent. These samples were previously utilized and are described in detail in Burns et al. (2015). To reiterate these points, all research conformed to the Helsinki Declaration and was approved by the University of Minnesota Institutional Review Board, protocol 1310E44403. Tissue pairs were resected concurrently, rinsed with sterile water, flash frozen in liquid nitrogen, and characterized by staff pathologists. The criteria for selection were limited to the availability of patient-matched normal and tumor tissue specimens. Additional patient metadata are provided in Burns et al. (2015).

### Microbiome Characterization

The microbiome data used in the study was generated previously (Burns et al. (2015). Briefly, microbial DNA was extracted from patient-matched normal and tumor tissue samples using sonication for lysis and the AllPrep nucleic acid extraction kit (Qiagen, Valencia, Calif.). The V5-V6 regions of the 16S rRNA gene were PCR amplified with the addition of barcodes for multiplexing using the forward and reverse primer sets V5F and V6R from Cai et al. (2013). The barcoded amplicons were pooled and Illumina adapters were ligated to the reads. A single lane on an Illumina MiSeq instrument was used (250 cycles, paired-end) to generate 16S rRNA gene sequences. The sequencing resulted in approximately 10.7 million total reads passing quality filtering in total, with a mean value of 121,470 quality reads per sample. The forward and reverse read pairs were merged using the USEARCH v7 program ‘fastq_mergepairs’, allowing stagger, with no mismatches allowed (66). OTUs were picked using the closed-reference picking script in QIIME v1.7.0 using the Greengenes database (August 2013 release) (Caporase et al., 2010; Navas-Molina at al., 2013; DeSantis et al., 2006). The similarity threshold was set at 97%, reverse-read matching was enabled, and reference-based chimera calling was disabled.

### Exome Sequence Data Generation

Genomic DNA samples were quantified using a fluorometric assay, the Quant-iT PicoGreen dsDNA Assay Kit (Life Technologies, Grand Island, N.Y.). Samples were considered passing quality control (QC) if they contained greater than 300 nanograms (ng) of DNA and display an A260:280 ratio above 1.7. Full workflow details for library preparation are outlined in the Nextera Rapid Capture Enrichment Protocol Guide (Illumina, Inc., San Diego, Calif.). In brief, libraries for Illumina next-generation sequencing were generated using Nextera library creation reagents (Illumina, Inc., San Diego, Calif.). A total of 50 ng of genomic DNA per sample were used as input for the library preparation. The DNA was tagmented (simultaneously tagged and fragmented) using Nextera transposome based fragmentation and transposition as part of the Nextera Rapid Capture Enrichment kit (Illumina, Inc., San Diego, Calif.). This process added Nextera adapters with complementarity to PCR primers containing sequences that allow addition of Illumina flow cell adapters and dual-indexed barcodes. The tagmented DNA was amplified using dual indexed barcoded primers. The amplified and indexed samples were pooled (8 samples per pool) and quantified to ensure appropriate DNA concentrations and fragment sizes using the fluorometric PicoGreen assay and the Bioanalyzer High-Sensitivity DNA Chip (Agilent Technologies, Santa Clara, Calif.). Libraries were considered to pass QC as long as they contained more than 500 ng of DNA and had an average peak size between 200-1000 base pairs. For hybridization and sequence capture, 500 nanograms of amplified library was hybridized to biotinylated oligonucleotide probes complementary to regions of interest at 58° C. for 24 hours. Library-probe hybrids were captured using streptavidin-coated magnetic beads and subjected to multiple washing steps to remove non-specifically bound material. The washed and eluted library was subjected to a second hybridization and capture to further enrich target sequences. The captured material was then amplified using 12 cycles of PCR. The captured, amplified libraries underwent QC using a Bioanalyzer, and fluorometric PicoGreen assay. Libraries were considered to pass QC as long as they contained a DNA concentration greater than 10 nM and had an average size between 300-400 base pairs. Libraries were hybridized to a paired end flow cell at a concentration of 10 pM and individual fragments were clonally amplified by bridge amplification on the Illumina cBot (Illumina, Inc., San Diego, Calif.). Eleven lanes on an Illumina HiSeq 2000 (Illumina, Inc., San Diego, Calif.) were required to generate the desired sequences. Once clustering was complete, the flow cell was loaded on the HiSeq 2000 and sequenced using Illumina's SBS chemistry at 100 bp per read. Upon completion of read 1, base pair index reads were performed to uniquely identify clustered libraries. Finally, the library fragments were resynthesized in the reverse direction and sequenced from the opposite end of the read 1 fragment, thus producing the paired end read 2. Full workflow details are outlined in Illumina's cBot User Guide and HiSeq 2000 User Guides. Base call (.bcl) files for each cycle of sequencing were generated by Illumina Real Time Analysis (RTA) software. The base call files and run folders were then exported to servers maintained at the Minnesota Supercomputing Institute. Primary analysis and de-multiplexing was performed using Illumina's CASAVA software 1.8.2. The end result of the CASAVA workflow was de-multiplexed FASTQ files that were utilized in subsequent analysis for read QC, mapping, and mutation calling.

### Exome Data Analysis

The exome sequence data contained approximately 4.2 billion reads in total following adapter removal and quality filtering, inclusive of forward and reverse reads, with a mean value of 47.8 million high-quality reads per sample. The raw reads were assessed using FastQC v0.11.2 and the Nextera adapters removed using cutadapt v1.8.1 (Babraham Biointomatics; Martin, 2011). Simultaneously, cutadapt was used to trim reads at bases with quality scores less than 20. Reads shorter than 40 bases were excluded. The trimmed and filtered read pairs were aligned and mapped to the human reference genome (hg19) using bwa v0.7.10 resulting in a bam file for each patient sample (Li et al., 2009). These files were further processed to sort the reads, add read groups, correct the mate-pair information, and mark and remove PCR duplicates using picard tools v1.133 and samtools v0.1.18 (Broad Institute Picard Tools; Lie et al., 2009). Tumor-specific mutations were identified using FreeBayes v0.9.14-24-gc292036 (Gaurisin et al., 2012). Following these steps, 94.0% of the remaining read pairs mapped to the reference genome, hg19. Specifically, SNPs only were assessed and a minimum coverage at each identified mutation position of more than 30× was required in both the patient normal and tumor samples. These mutations were filtered to only include those that were within protein-coding regions and compiled into a single vcf file. This vcf file was assessed using SNPeffect v4.1 K (2015-09-0) in order to predict the potential impact of each of the mutations (Cingolani et al., 2012). Based on these results, the mutations were grouped into three categories: (1) total mutations (2) non-synonymous mutations and (3) loss of function (LoF) mutations. The total mutations group is self-explanatory. The non-synonymous mutations included all the mutations in the total mutations group that were non-silent. The LoF group only included those mutations that resulted in a premature stop codon, a loss of a stop codon, or a frameshift mutation.

### Joint Analysis of Microbiome and Exome Data

Taxa that differentiated patients with or without LoF mutation were identified using LEfSe (Segata et al., 2011). All the taxa with a LDA score (log 10)>2 were included in the calculation of the risk indices, built to predict the presence or absence of a LoF mutation based on the OTU table collapsed at genus level. To build the risk index, the relative abundances (arcsine square root transformed) of the taxa associated with the LoF mutation (based on the LEfSe output) were summed and the relative abundances of the taxa associated with no mutation (based on the LEfSe output) were summed. The use of the unweighted sum in the risk index, rather than relying on the regression coefficients from LDA, is a simple way to control the degree of flexibility of the model when training on small sample sizes (Muntassier et al., 2016). Then the difference between these two sums was calculated, thereby obtaining a risk index. This procedure was repeated 44 times to obtain a risk index for each patient.

A leave-one-out procedure (also described above) was conducted to evaluate the taxa that differentiated patients with or without LoF mutation in the held-out patient, based on the LEfSe output of n−1 patients. In detail, the taxa that differentiated patients with or without LoF mutation were identified using LEfSe in the n−1 dataset. The relative abundances of the taxa associated with the LoF mutation (based on the LEfSe output of the n−1 dataset) were summed and the relative abundances of the taxa associated with no mutation (based on the LEfSe output of the n−1 dataset) were summed and were used to build the risk index in the held-out patient. In detail, the difference between these two sums was calculated to obtain the risk index of the held-out patient. This procedure was repeated 44 times, to produce a risk index in each of the held-out patients, based on the difference between the sum of the taxa associated with the absence of LoF mutation minus the sum of the taxa associated with the presence of the LoF mutation found in each of the n−1 datasets. The significance of the difference in risk indexes between the patients with LoF mutation and patients with LoF mutation for each gene was assessed using a Mann-Whitney U test and a permutation test, in which we permuted the labels for a given gene 999 times, each time deriving new held-out predictions of the risk indexes for each subject for that gene. Then the observed difference in means between the patients with LoF mutation and patients with LoF mutation risk index predictions using the method on the actual LoF mutation labels to the differences observed in the permutations to obtain an empirical P-value was compared. The resulting P-values were corrected using the false discovery rate (FDR) correction for multiple hypothesis tests.

Receiving Operating Characteristic (ROC) curves were plotted and the area under the curve (AUC) values computed on a dataset containing 10 sets of predictions and corresponding labels obtained from 10-fold cross-validation using ROCR package in R (Sing et al., 2005). A risk index threshold was also obtained that best predicts the presence or absence of LoF mutation with a leave-one-out cross-validation on the risk index. Each held-out sample was treated as a new patient on whom the optimal risk index cutoff was tested and subsequently refined to separate patients who had a LoF mutation and patient who did not have a LoF.

Correlation analysis was performed using SparCC on a reduced OTU table containing significant taxa identified using the above prediction methods collapsed to the genus level (Friedman et al., 2012). Pseudo p-values were calculated using 100 randomized sets. Networks of correlations were visualized using Cytoscape v3.1.0 (Shannon et al., 2003).

As this work is an extension of a previous study of the CRC-associated microbiome, each of the patients in this project have associated clinical data (Burns et al., 2015). A linear model was used to determine the extent to which any of these factors may correlate with mutation load. These included patient sex, tumor stage, patient age, patient body mass index (BMI), and microsatellite instability (MSI) status. None of these factors, alone or in combination, were found to significantly impact the mutational data, though it bears noting that MSI status was only available for a subset (13 out of 44) of the patients.

### Results

### Changes in the Microbiome Reflect Tumor Stage.

Whole-exome sequencing was performed on a set of 88 samples, comprised of 44 pairs of tumor and normal colon tissue sample from the same patient, with previously characterized tissue-associated microbiomes (Burns et al., 2015. The mutations in each of the tumors' protein-coding regions were identified relative to the patient-matched normal sample and annotated as either synonymous, non-synonymous, or loss-of-function (LoF) mutations. The mutations were collapsed by gene as well as by pathways using both Kyoto Encyclopedia of Genes and Genomes (KEGG) and pathway interaction database (PID) annotations (Kanehisa et al., 2000; Kanehisa et al., 2014; Kanehisa et al., 2015; Schaefer et al., 2009).

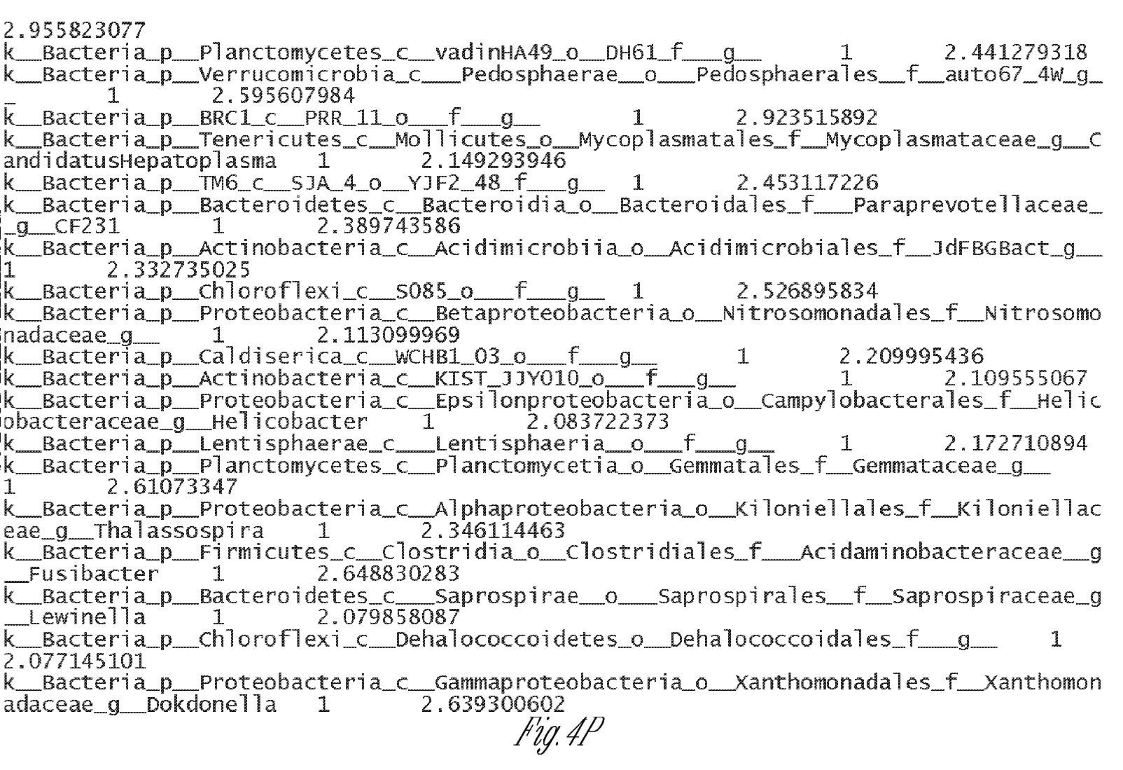

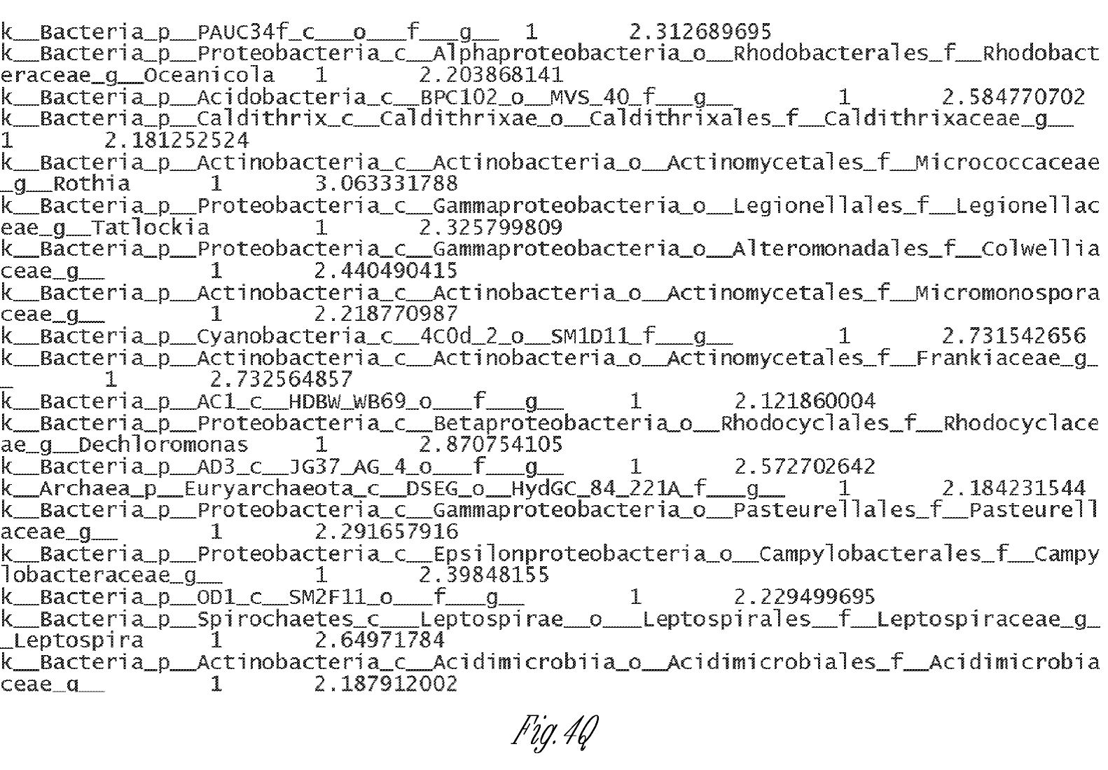

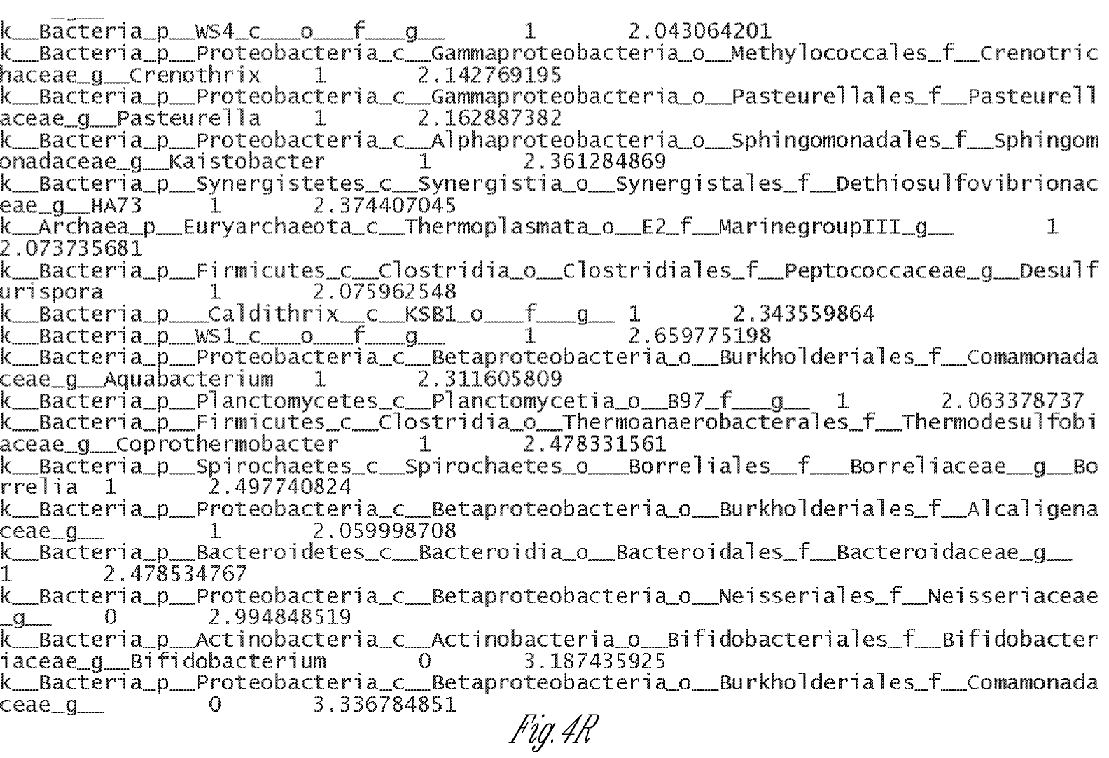

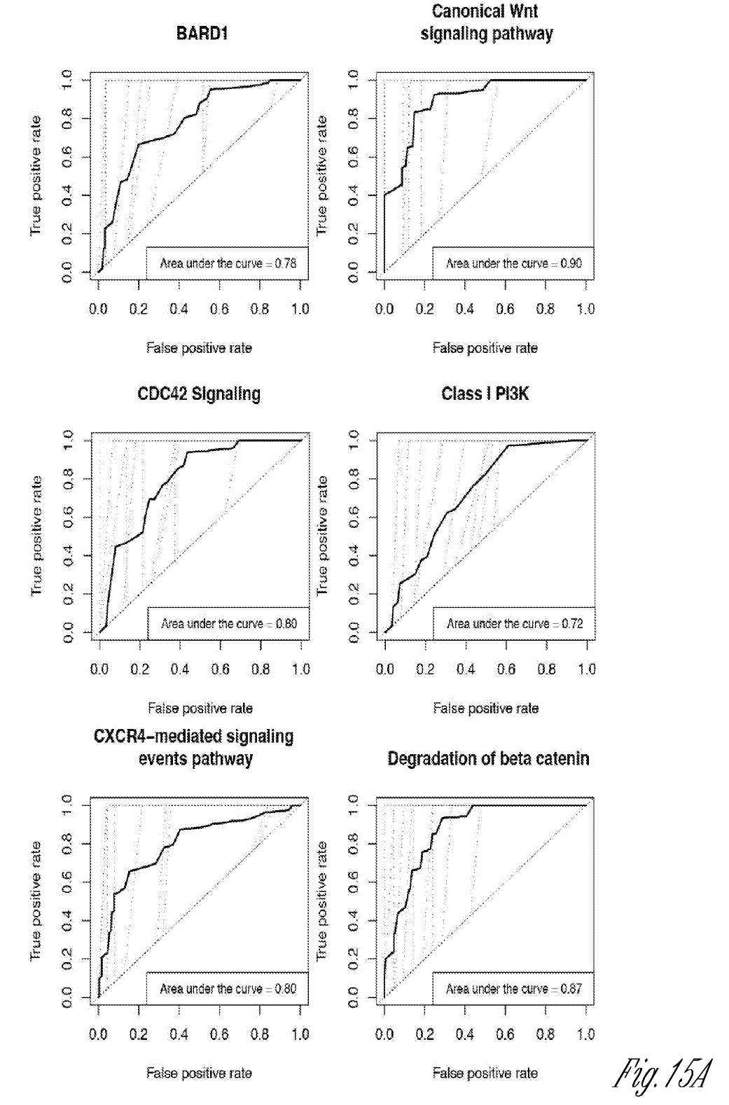

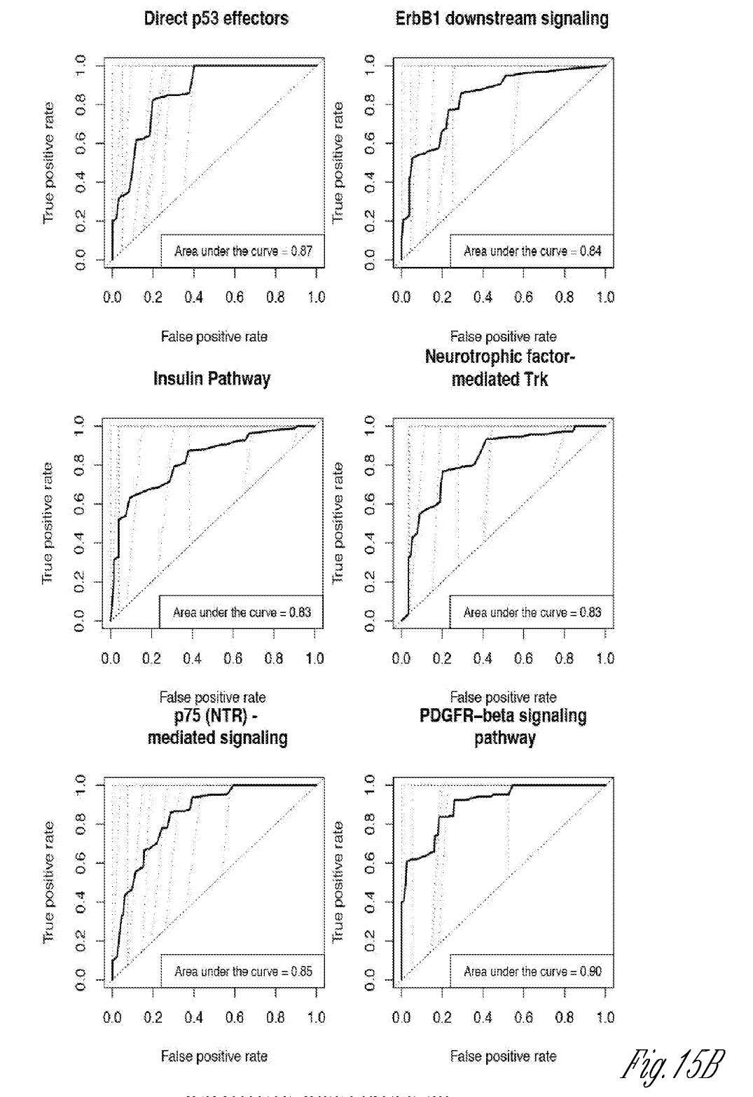

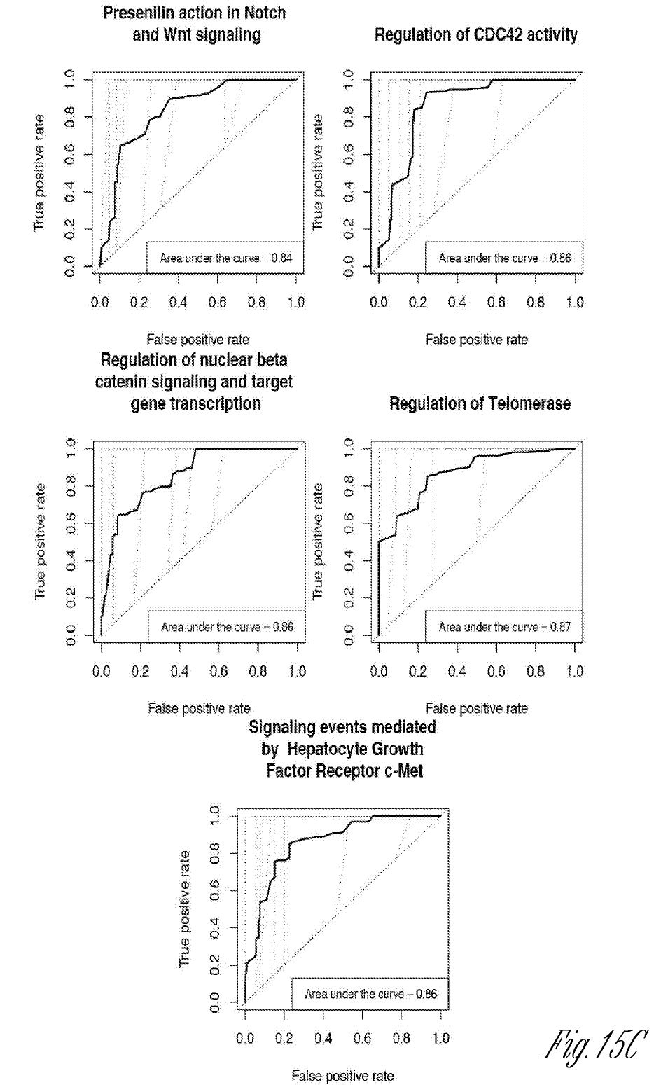

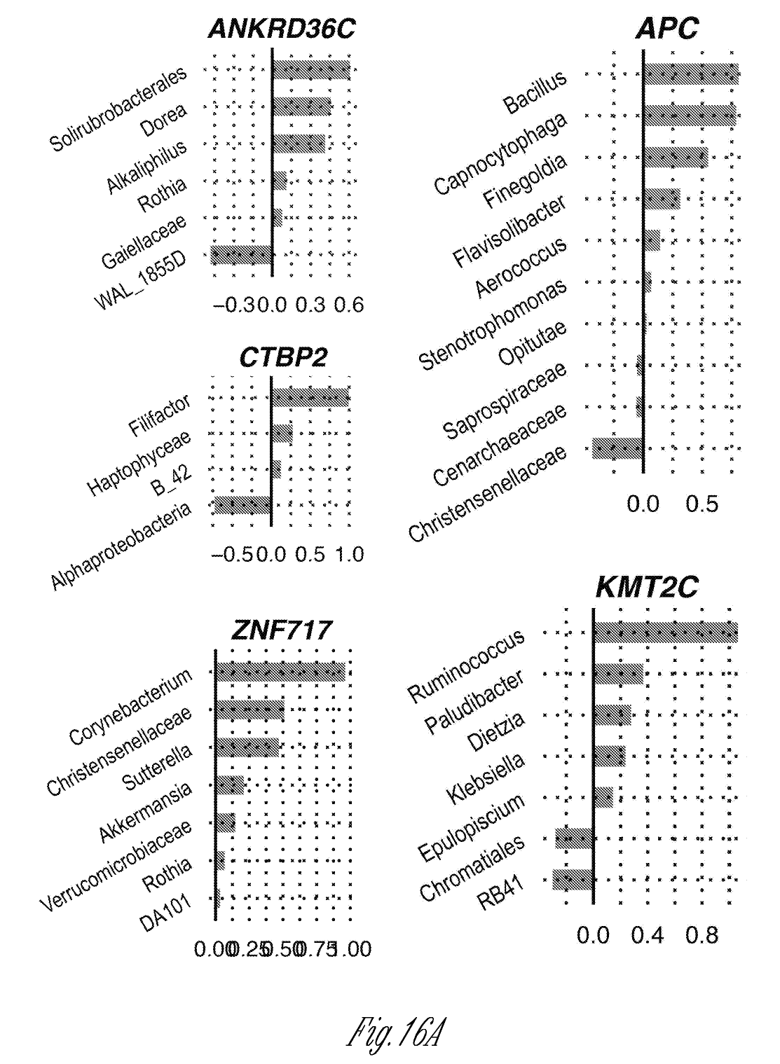

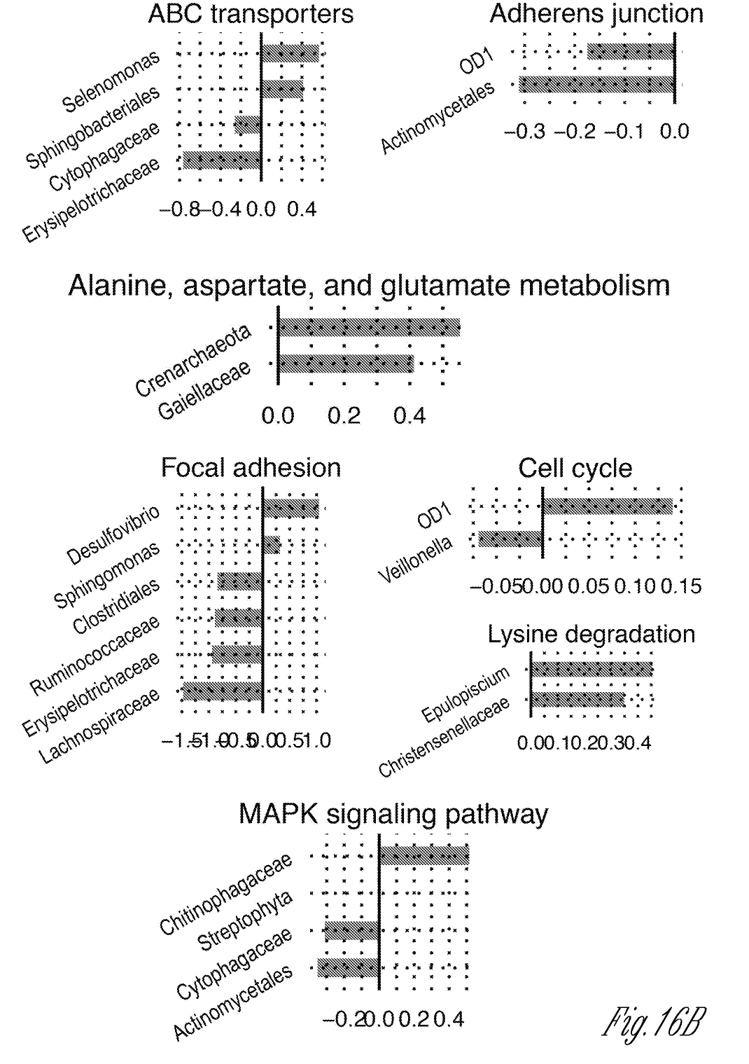

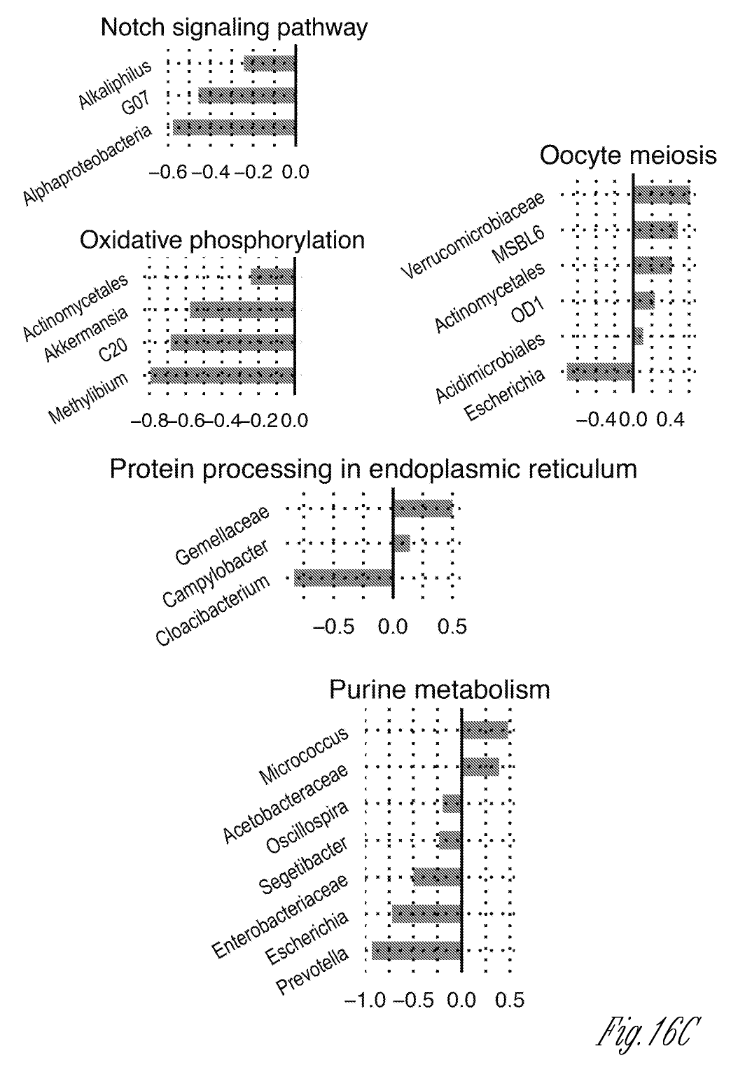

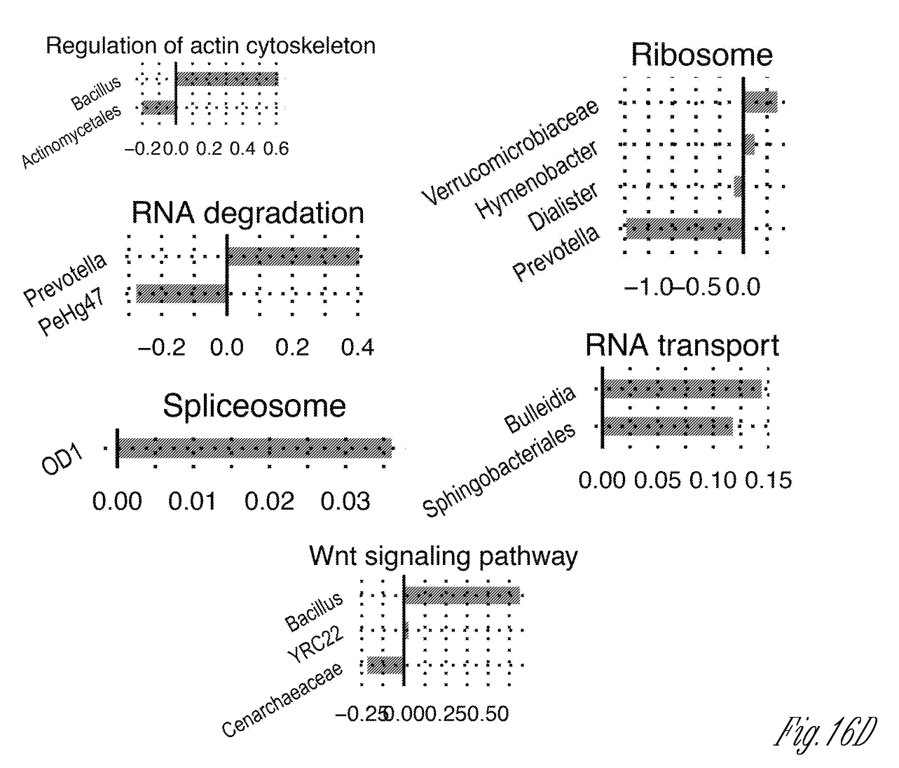

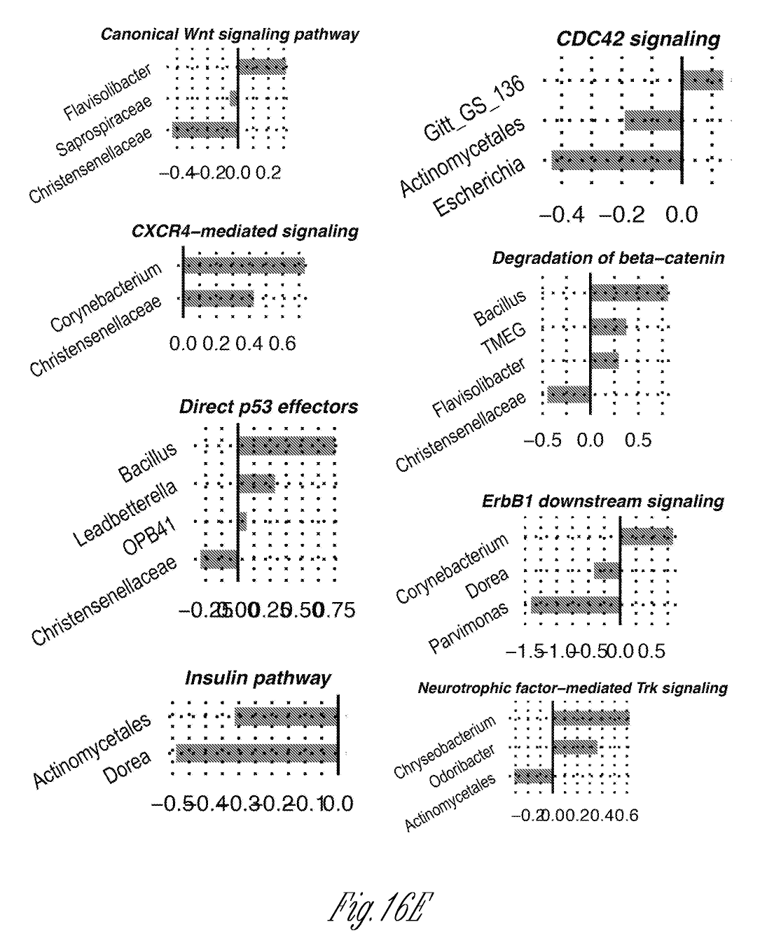

The relationship between microbial communities and tumor stage was investigated (FIG. 5). It was hypothesize that the structure and composition of the associated microbiome can be affected by relevant physiological and anatomical differences between the tumors at different stages that would provide different microenvironmental niches for microbes. The changes in the microbial communities surrounding each tumor were identified as a function of stage by grouping the tumors into low stage (stages 1-2) and high stage (stages 3-4) classes and linear discriminant analysis (LDA) effect size (LEfSe) was applied to the raw operational taxonomic unit (OTU) tables corresponding to these tumors (FIGS. 17-18) (Segata at al., 2011). The set of taxon abundances was transformed to generate a single value representing a risk index classifier for membership in the low-stage or high-stage group (FIG. 5A). To ascertain the error associated with these risk indices, a leave-one-out (LOO) cross-validation approach was applied. The LOO results were also used to generate receiver operating characteristic (ROC) curves and to calculate the area under the curve (AUC; see FIG. 5B). In addition, a permutation test was performed to assess the method's robustness (FIG. 18). Using this approach, it was demonstrate that the changes in abundances of 31 microbial taxa can be used to generate a classifier that distinguishes between low-stage and high-stage tumors at a fixed specificity of 80% and an accuracy of 77.5% (P=0.02 by Mann-Whitney U test, and P=0.007 by a permutation test; FIG. 18). The resulting changes seen in this analysis of the microbial communities that vary by tumor stage were similar to those found in previous studies, including one using a Chinese patient cohort (Nakatsu et al., 2015; Ahn et al., 2013). In both cases, there were significant changes among several taxa within the phylum Bacteroidetes, including Porphyromonadaceae, Paludibacter, and Cydobacteriaceae (FIGS. 5 and 18).

Tumor Mutations Correlate with Consistent Changes in the Proximal Microbiome.

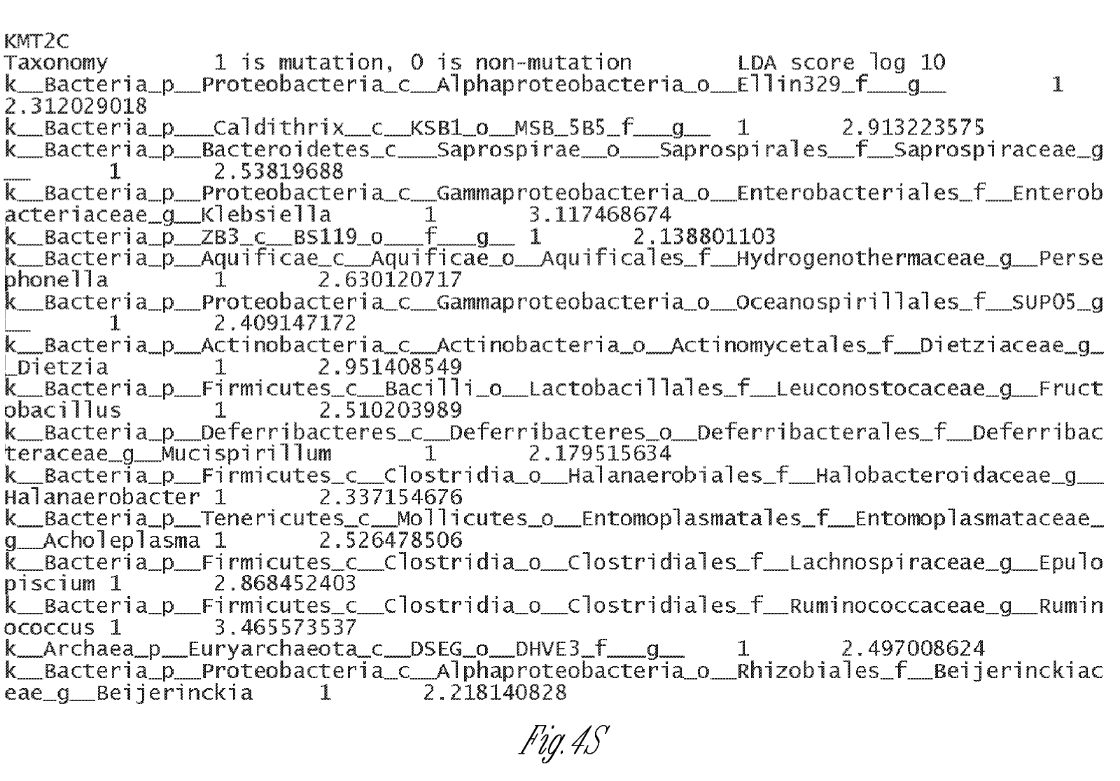

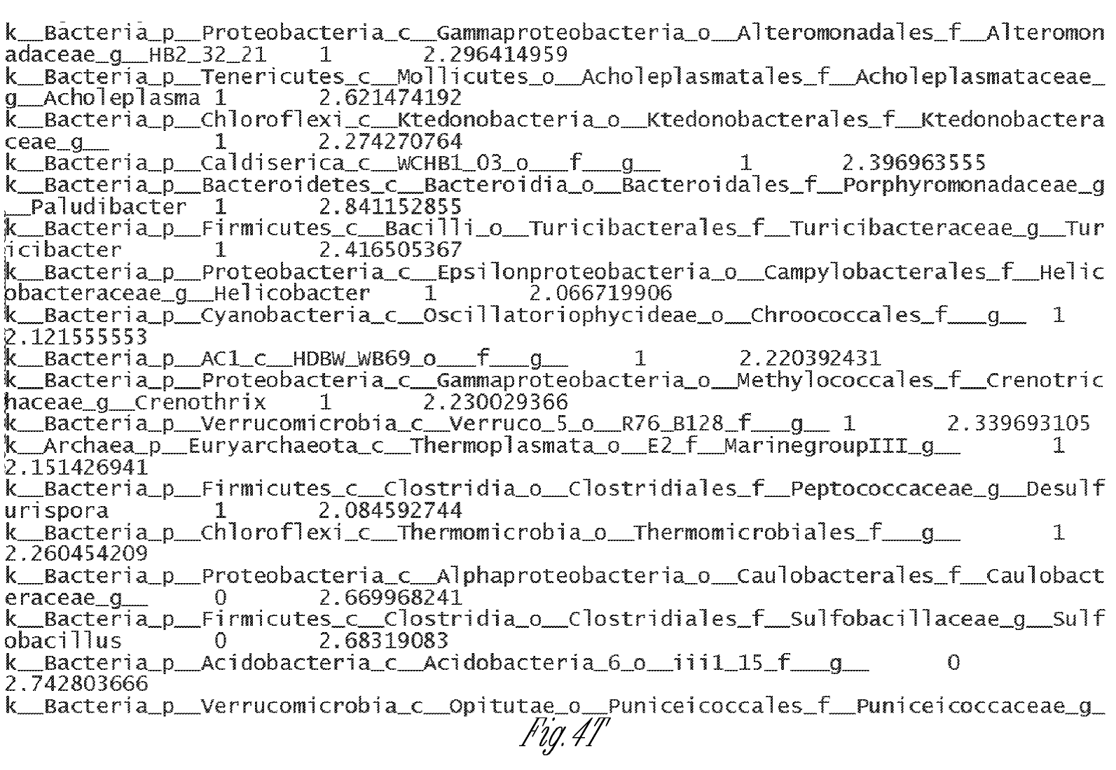

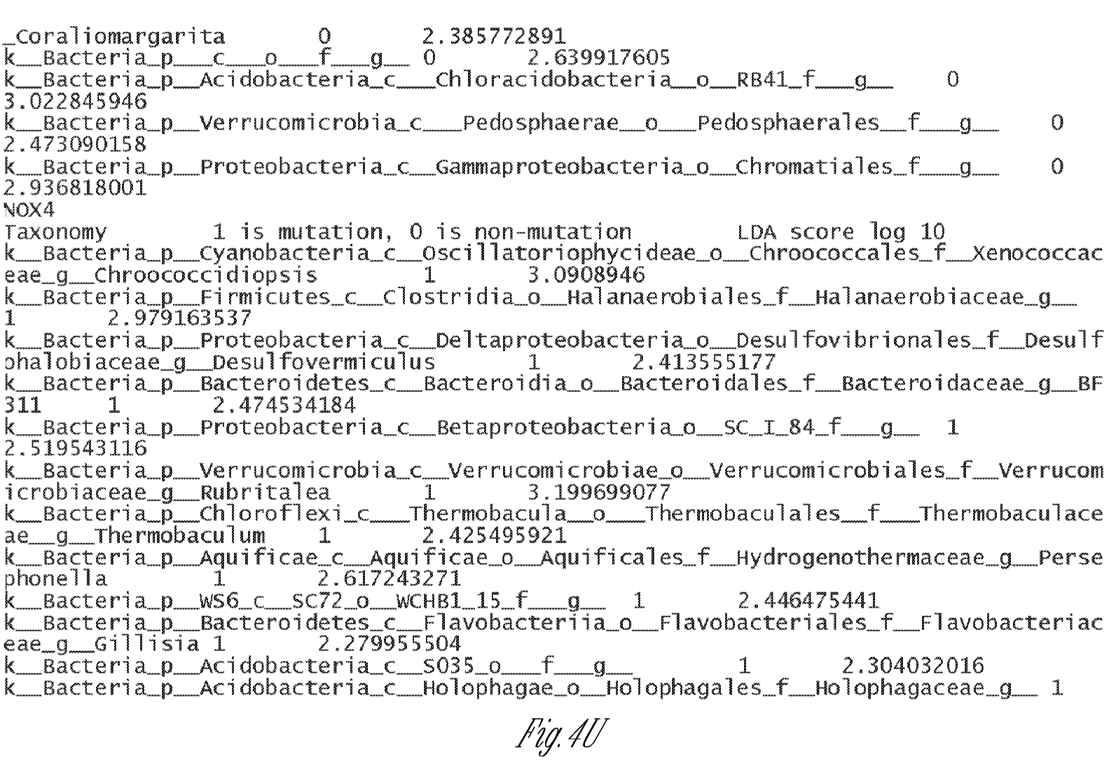

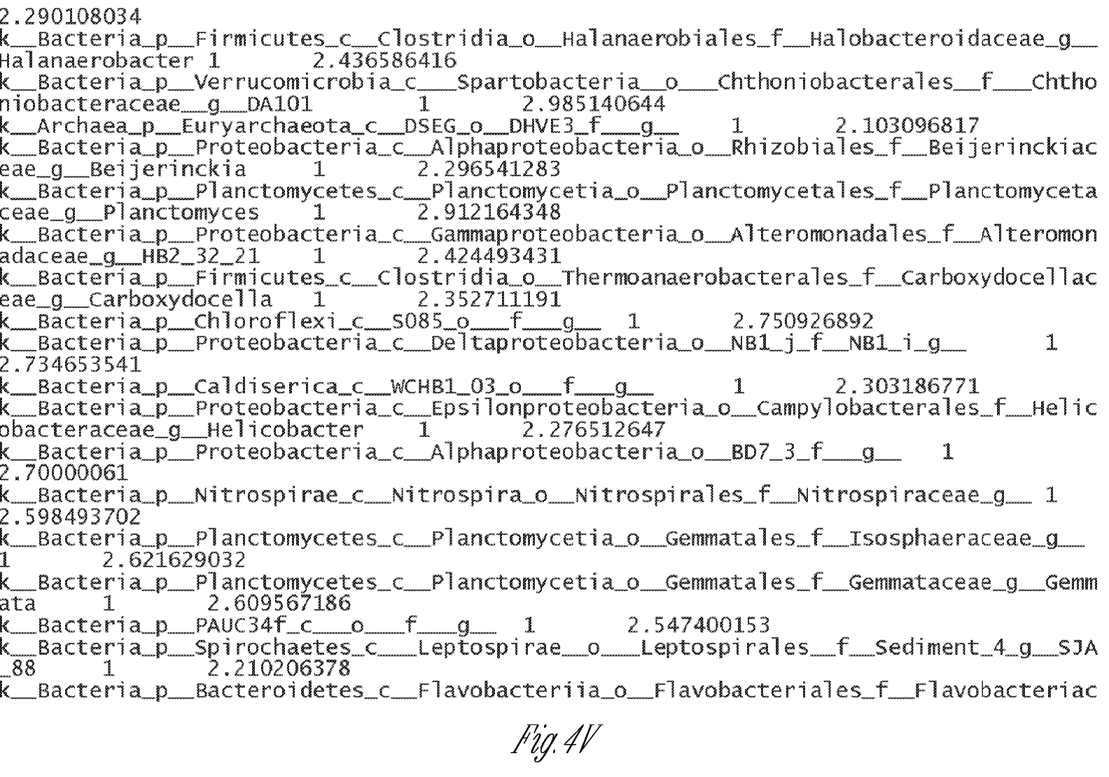

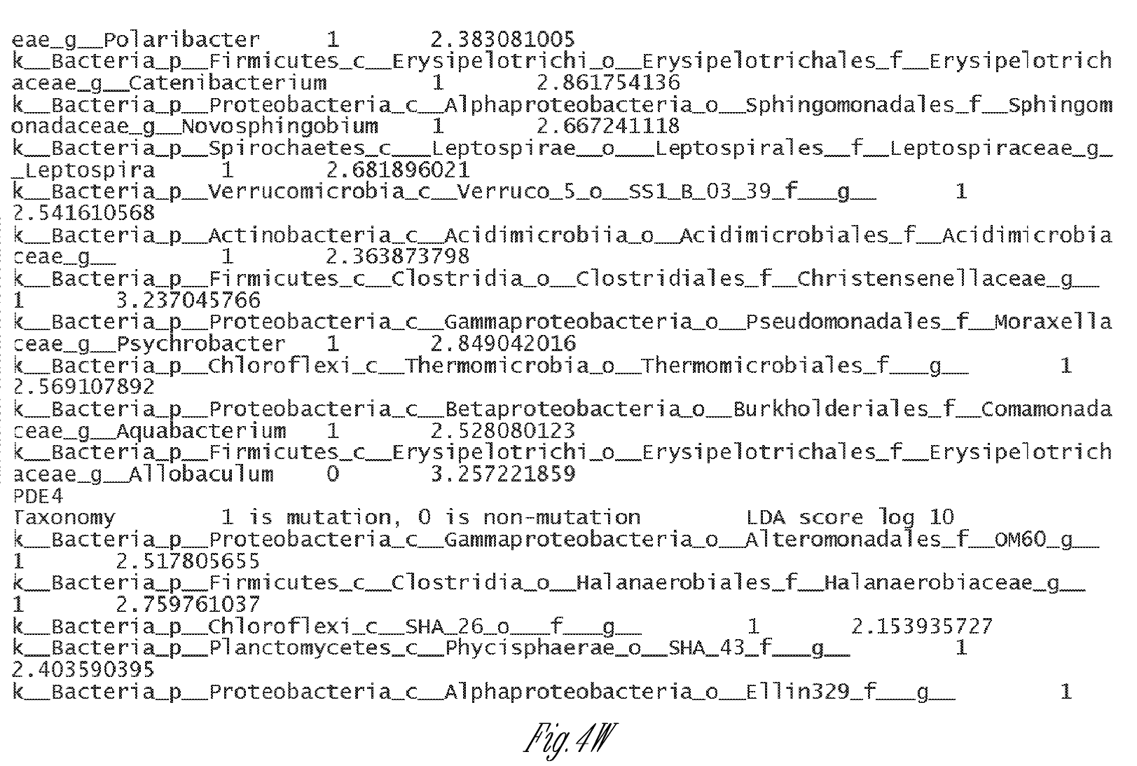

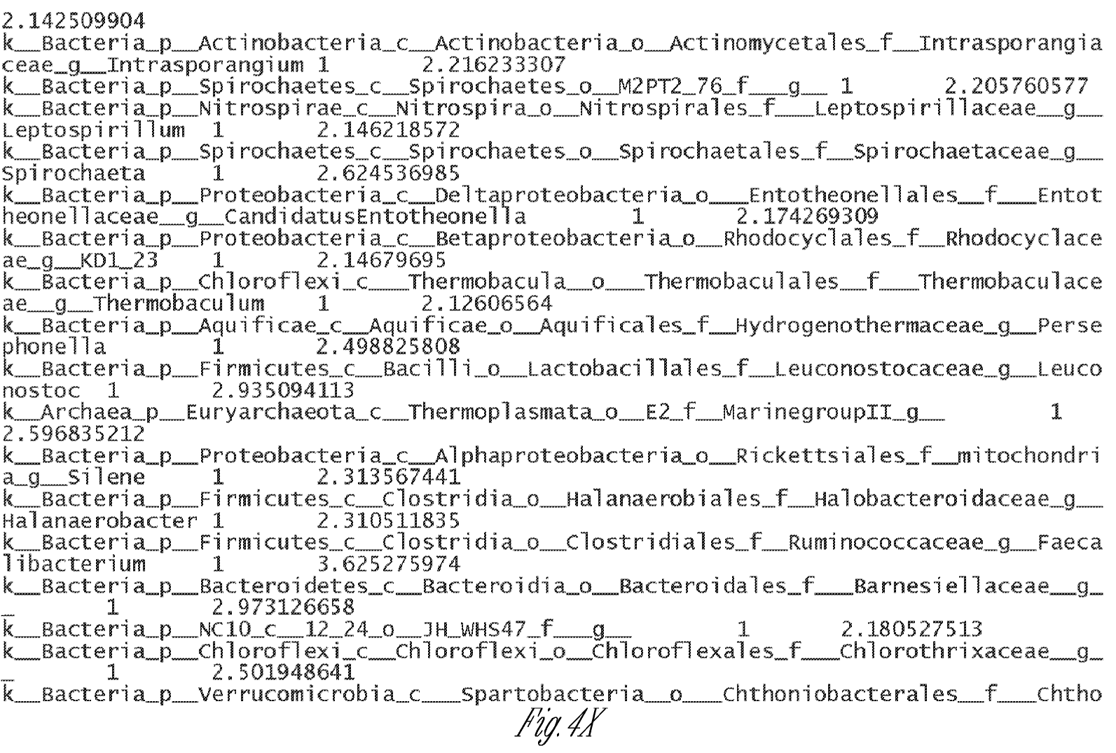

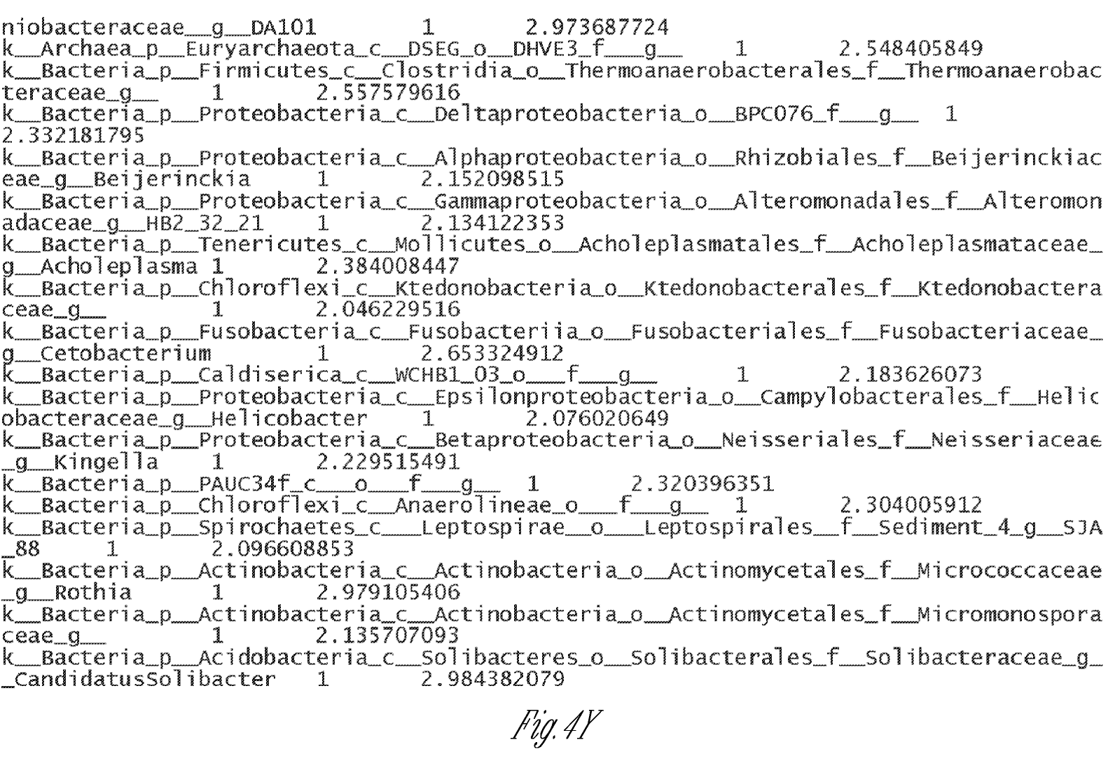

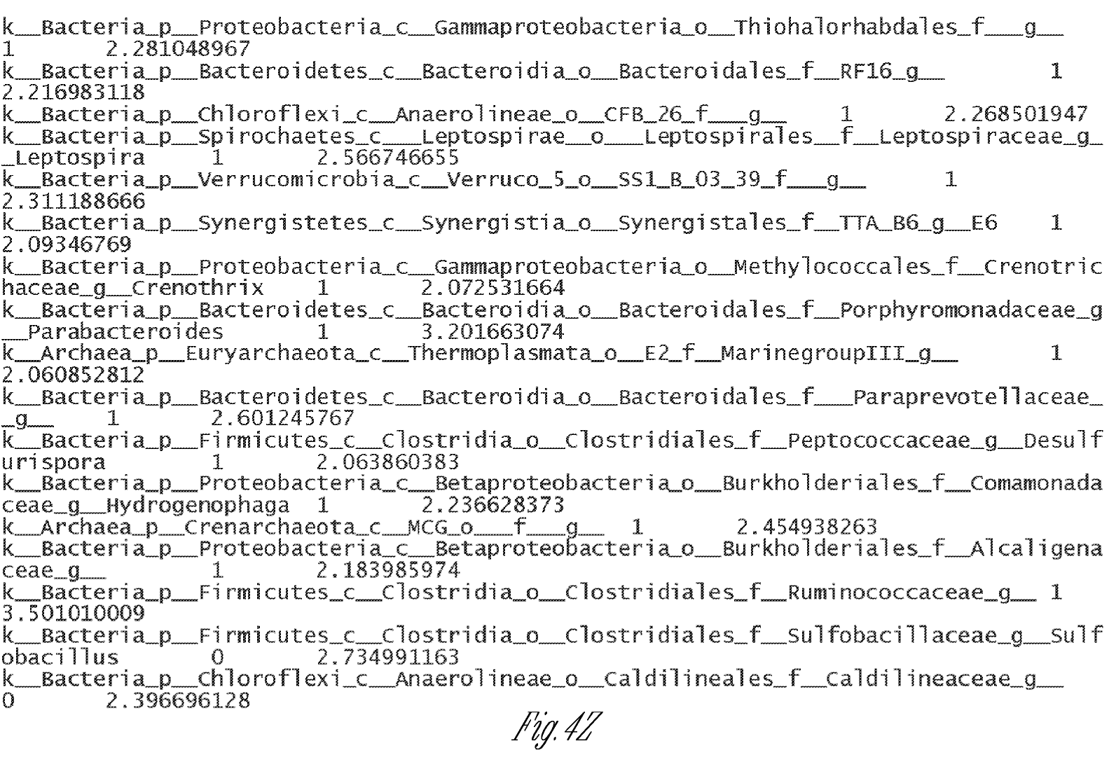

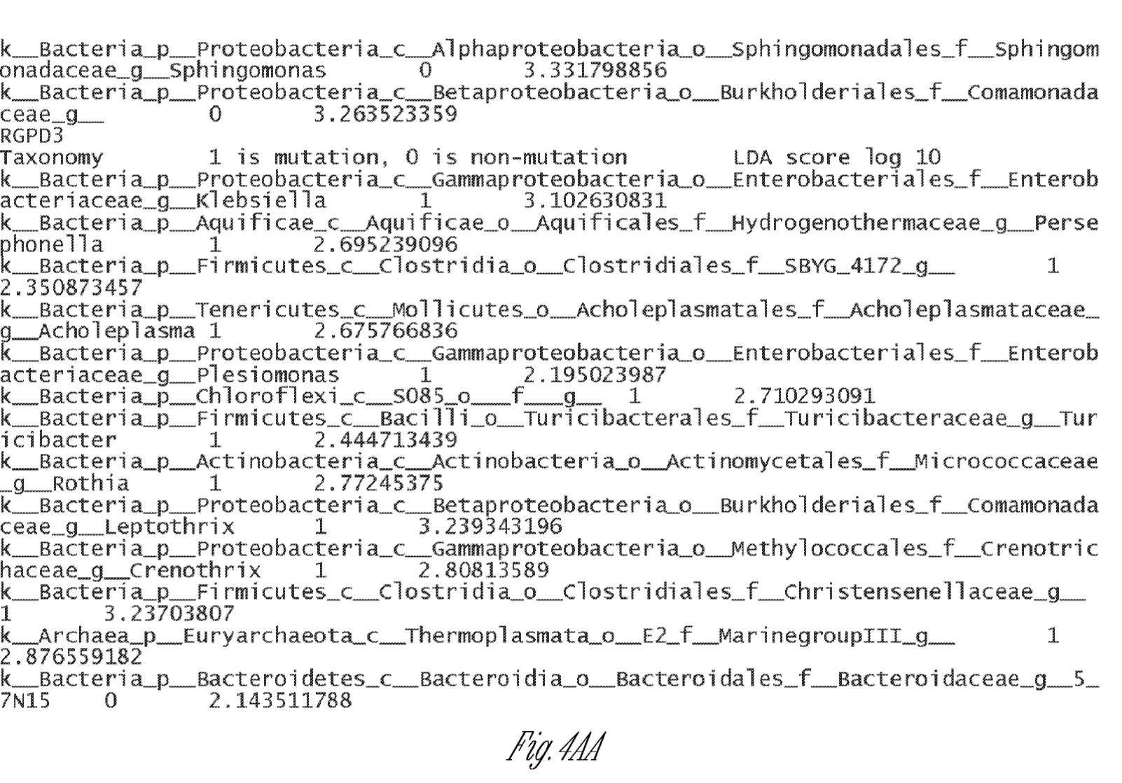

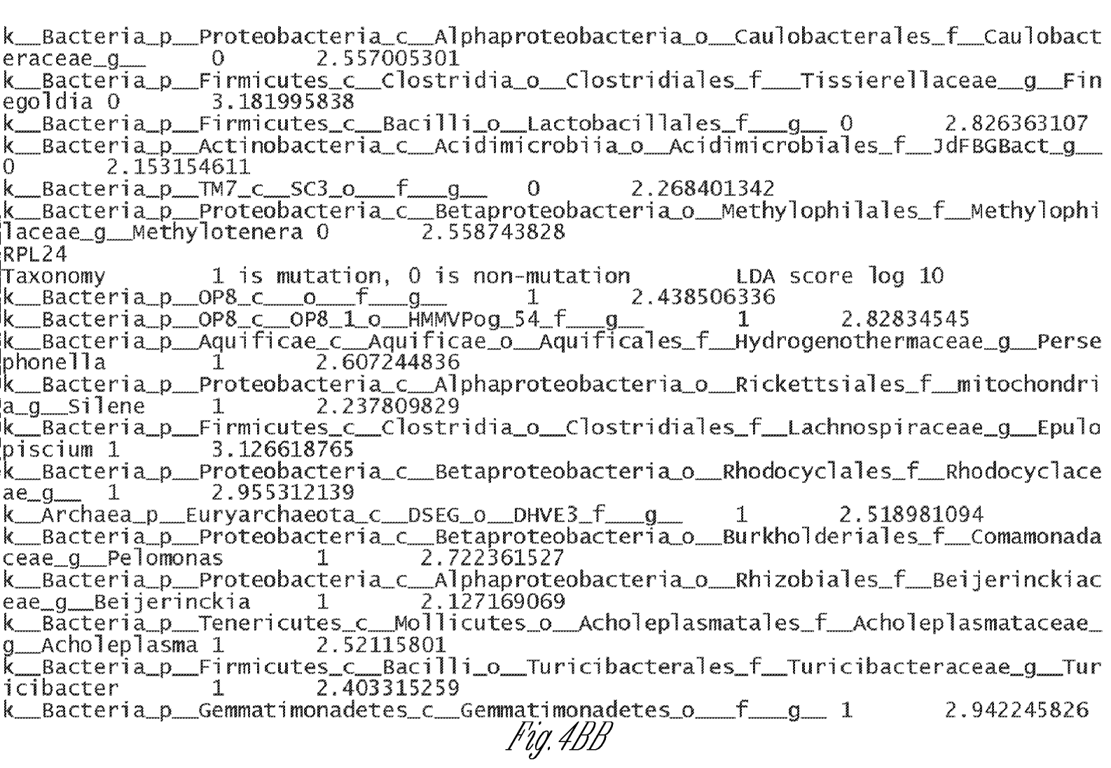

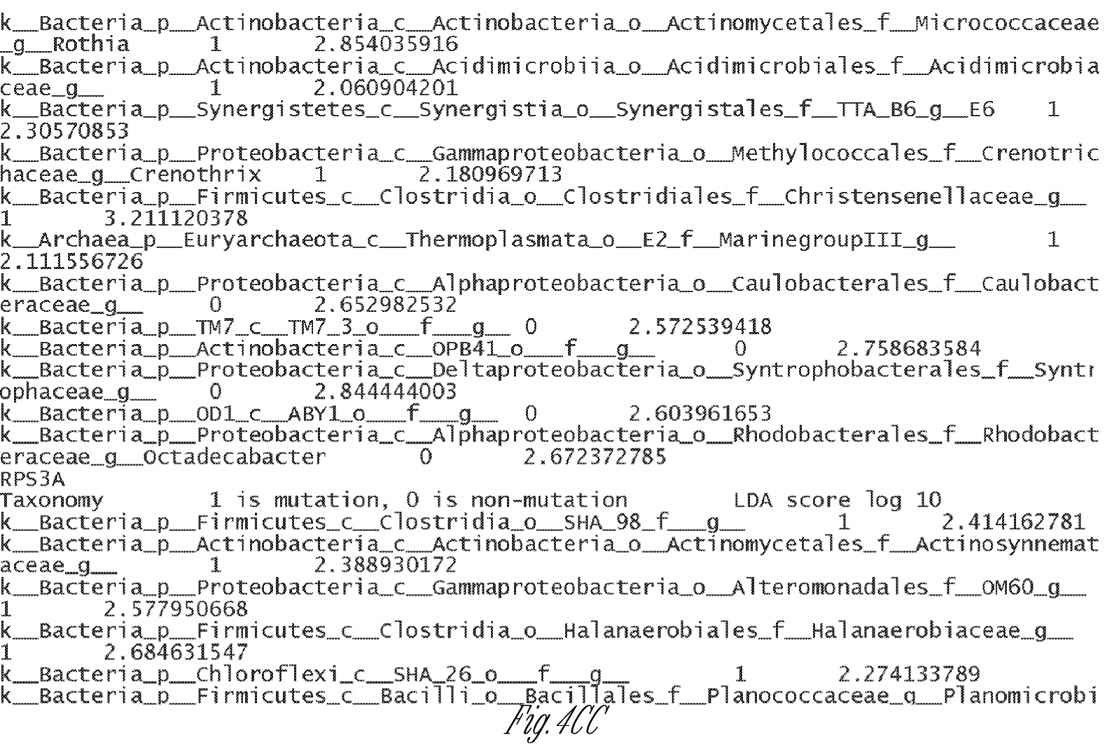

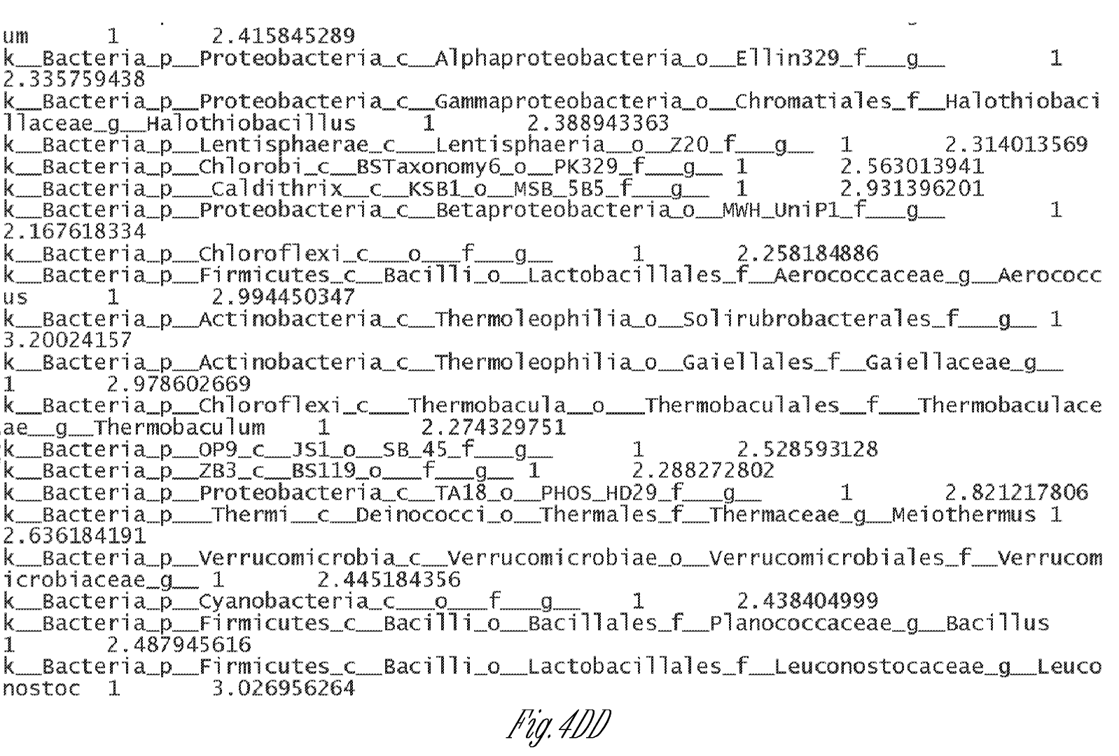

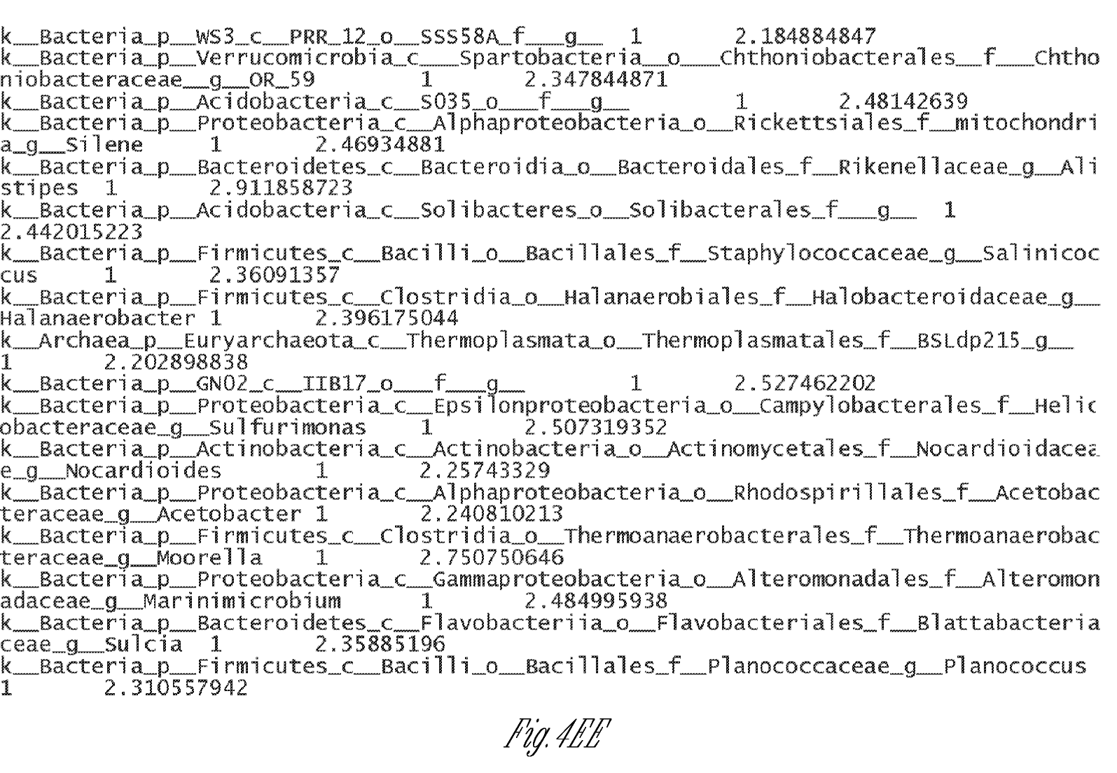

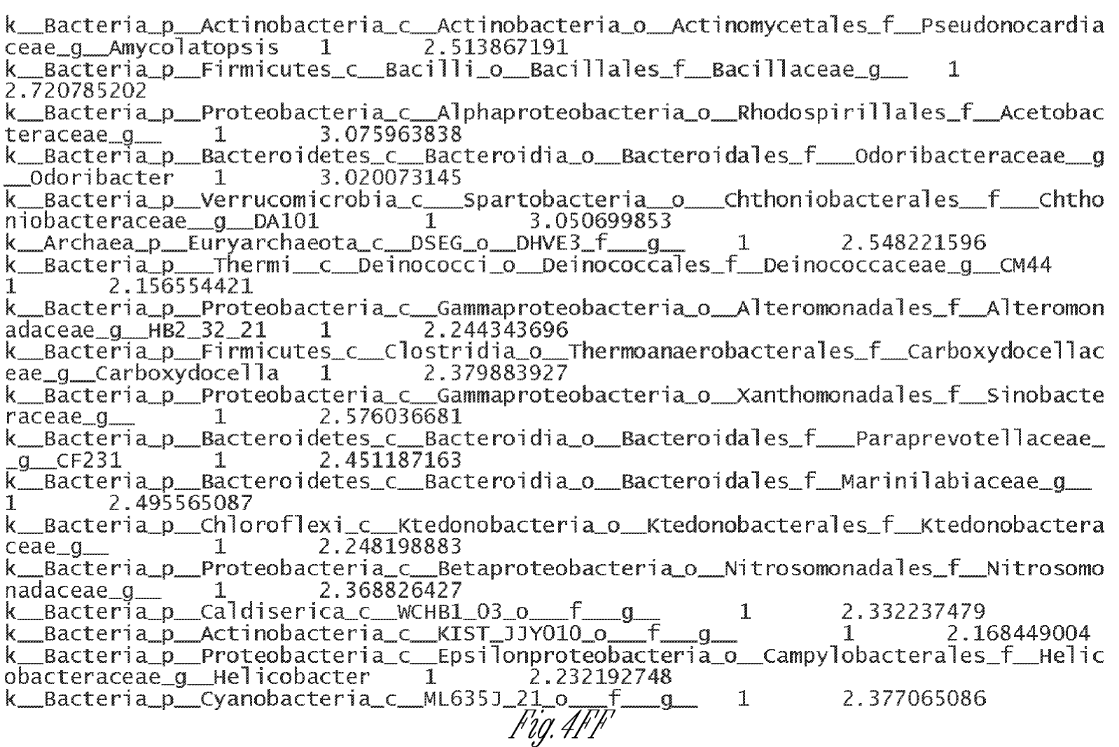

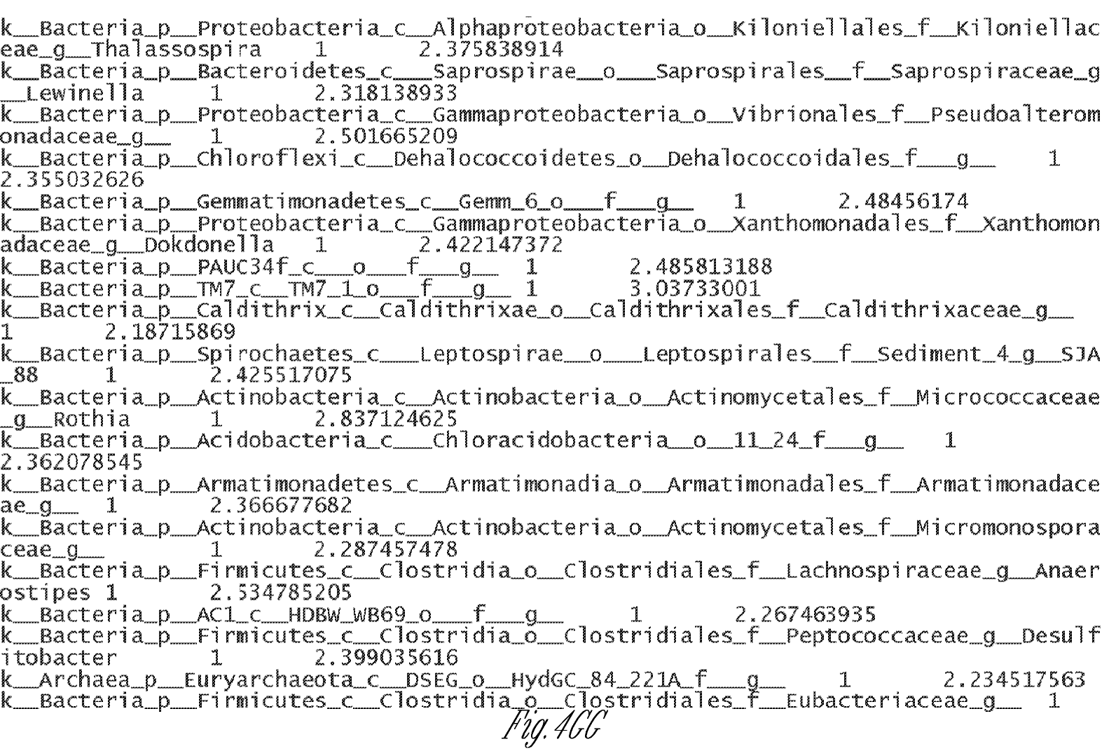

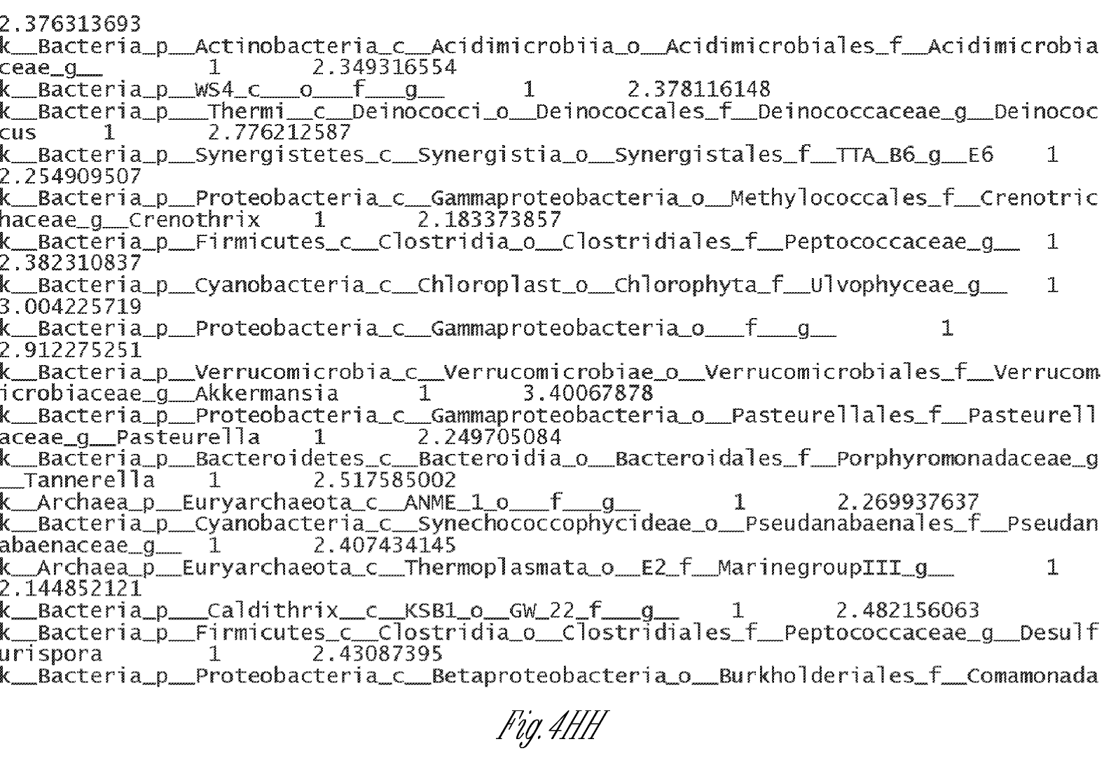

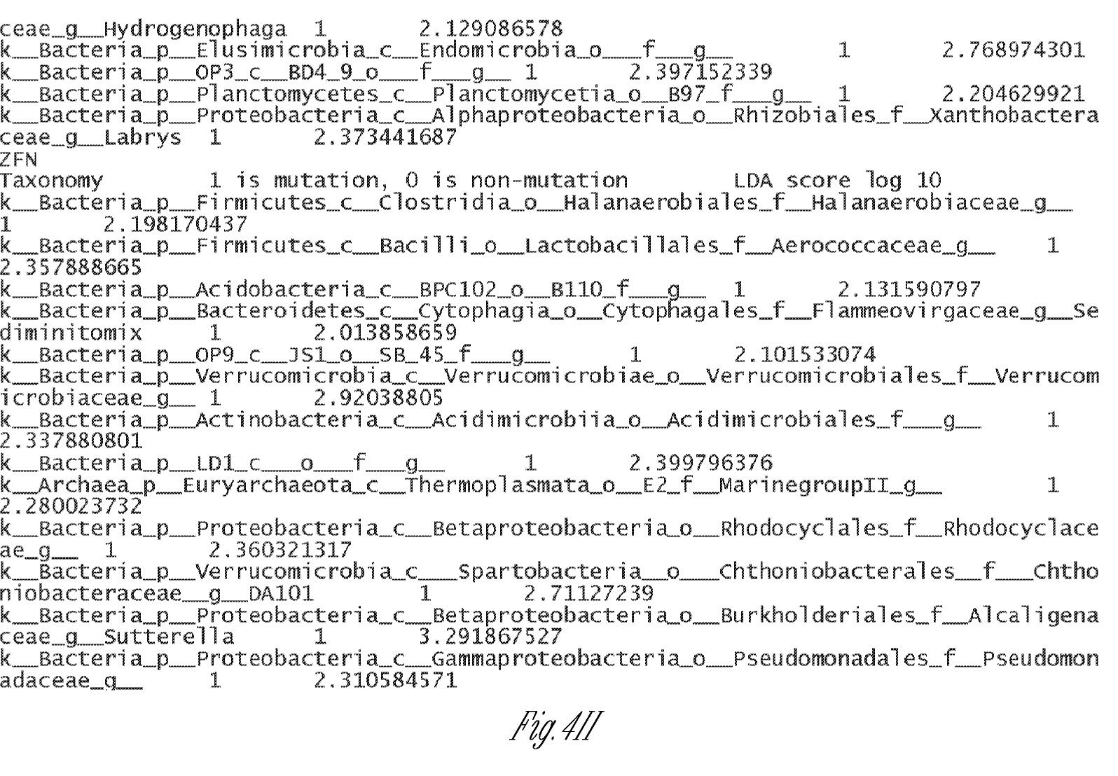

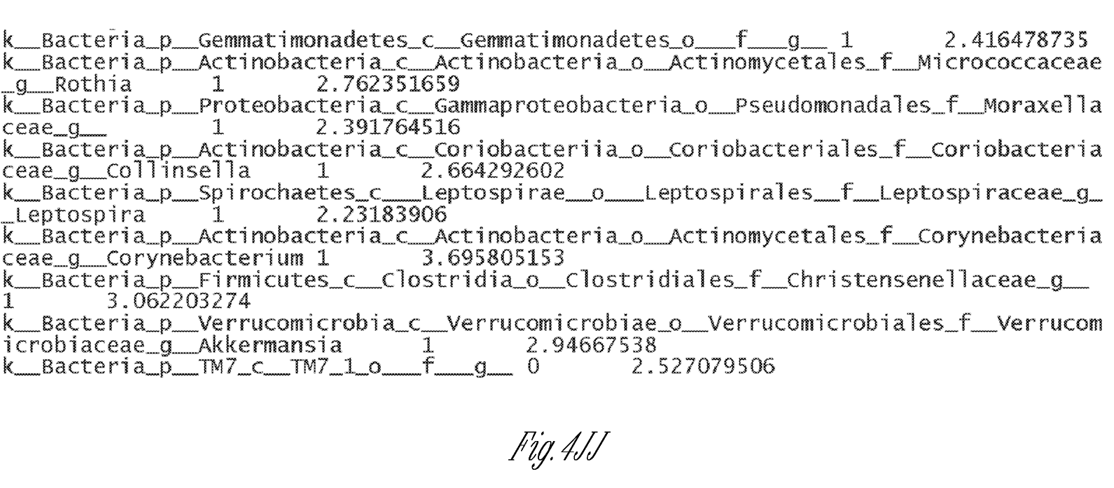

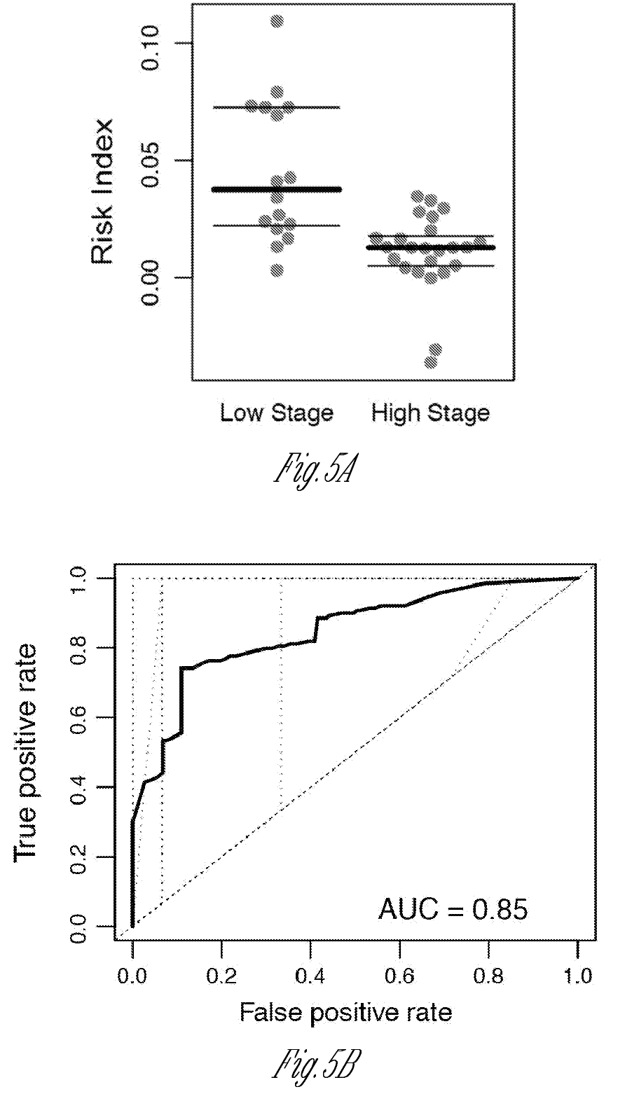

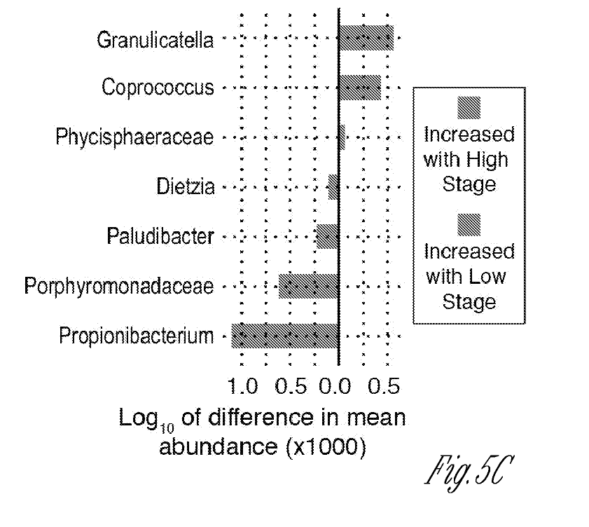

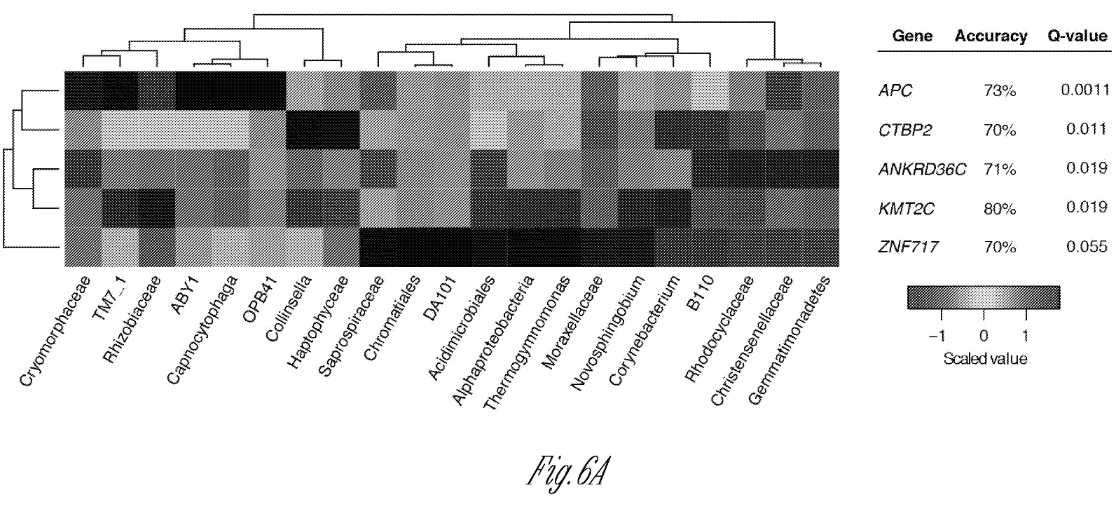

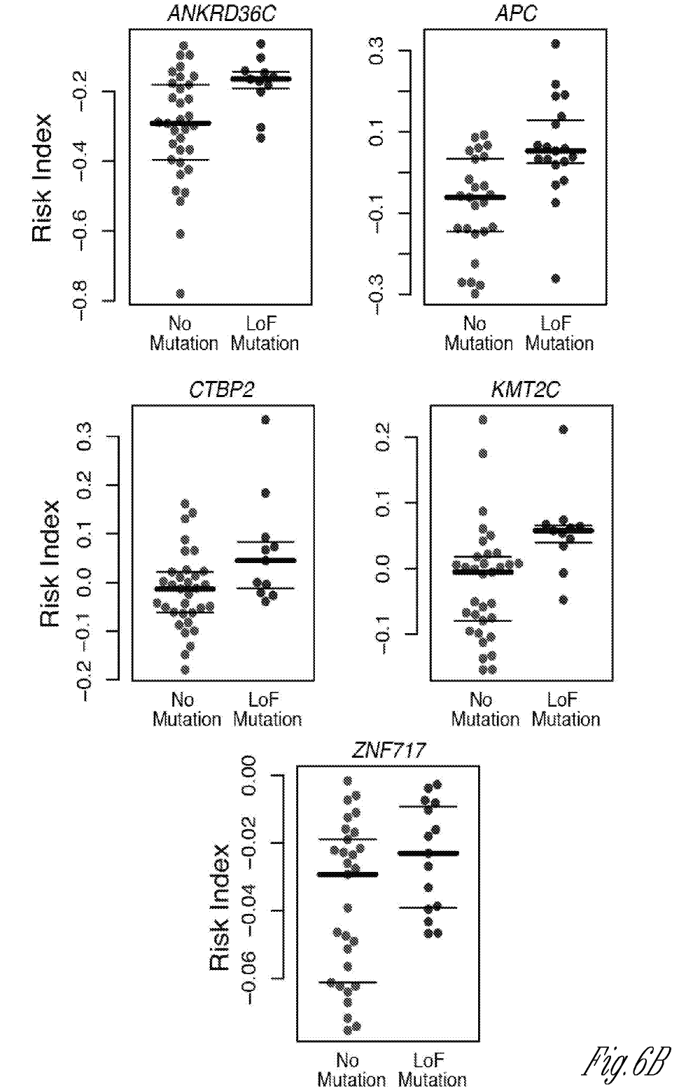

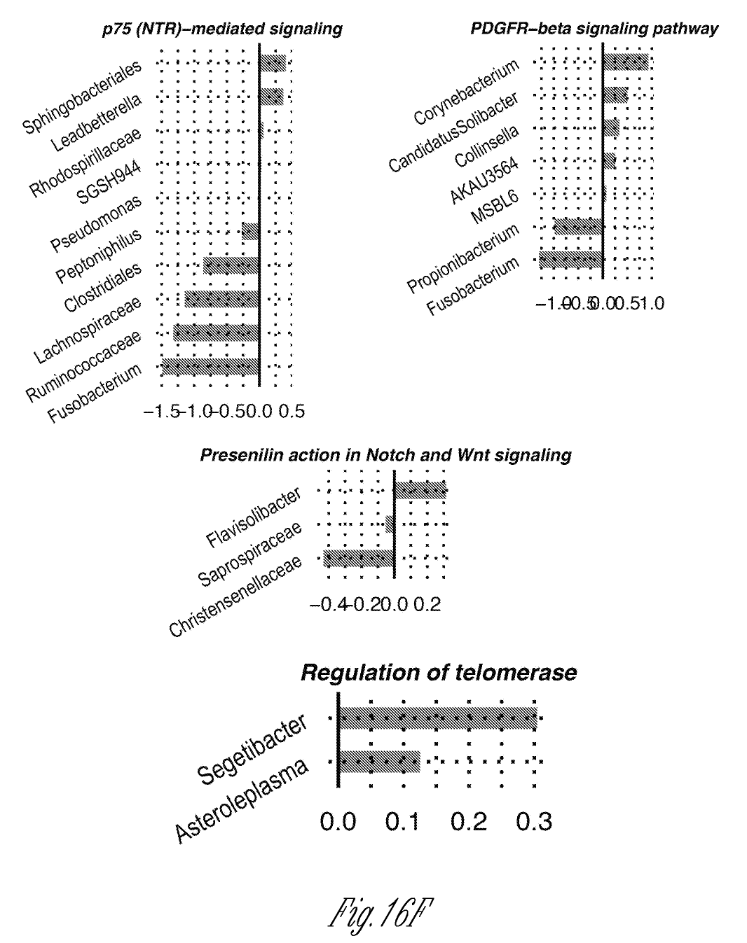

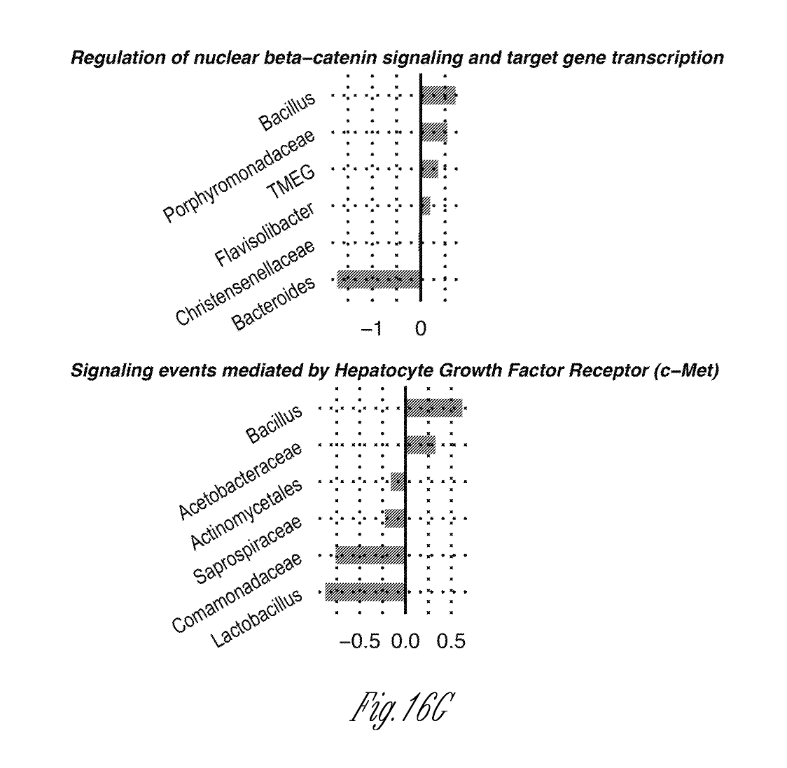

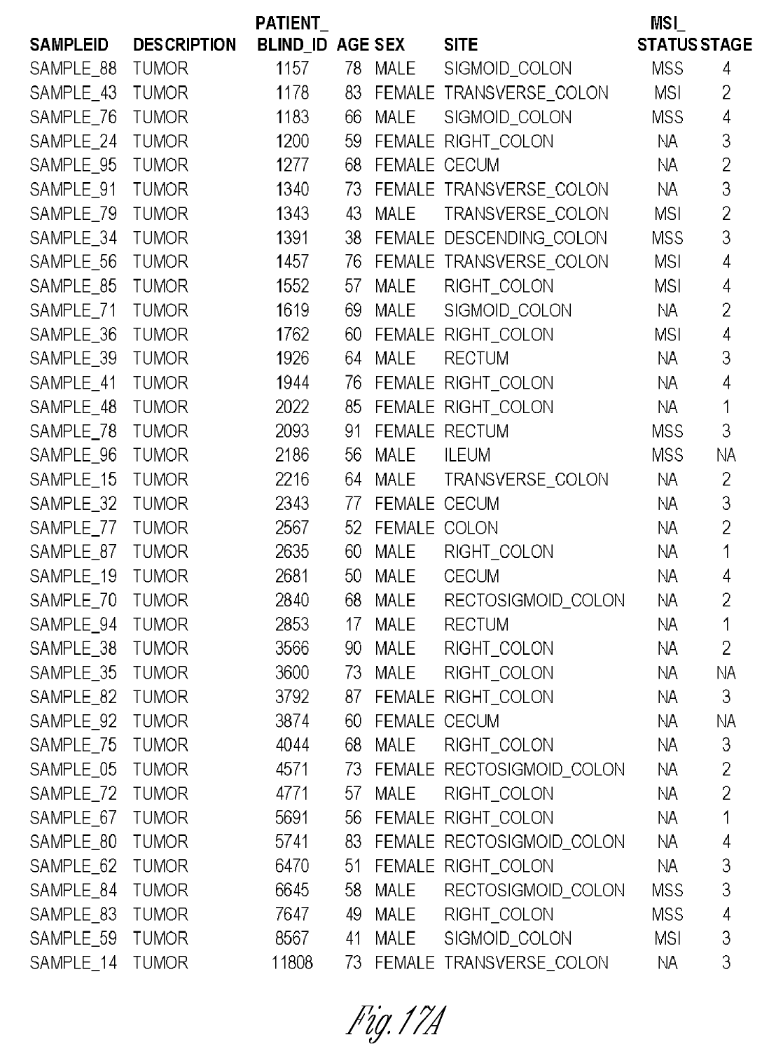

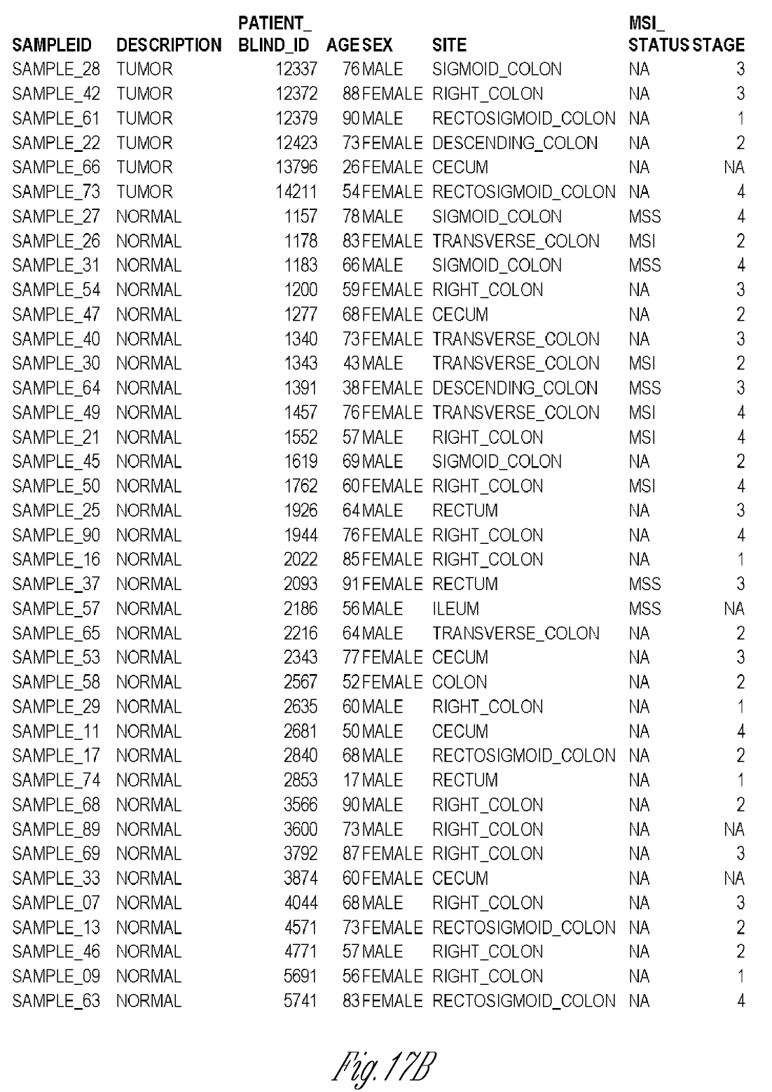

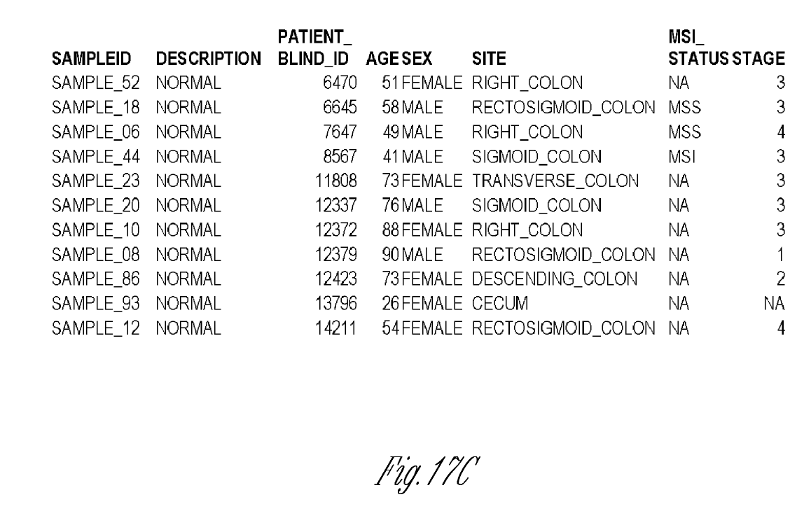

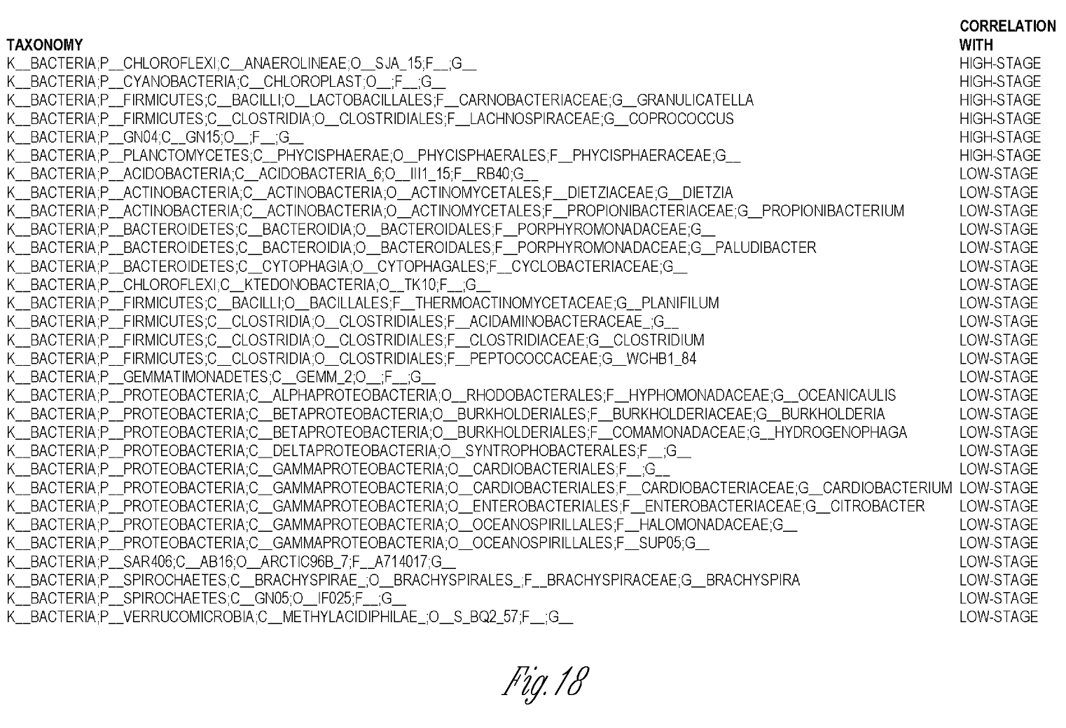

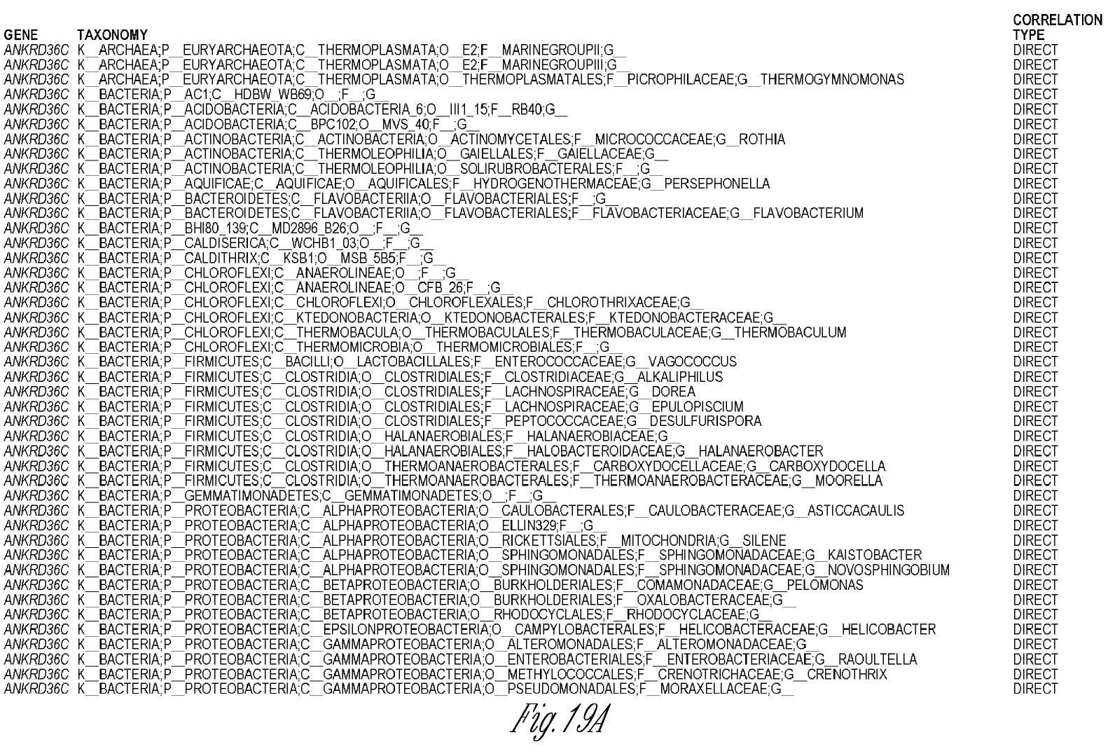

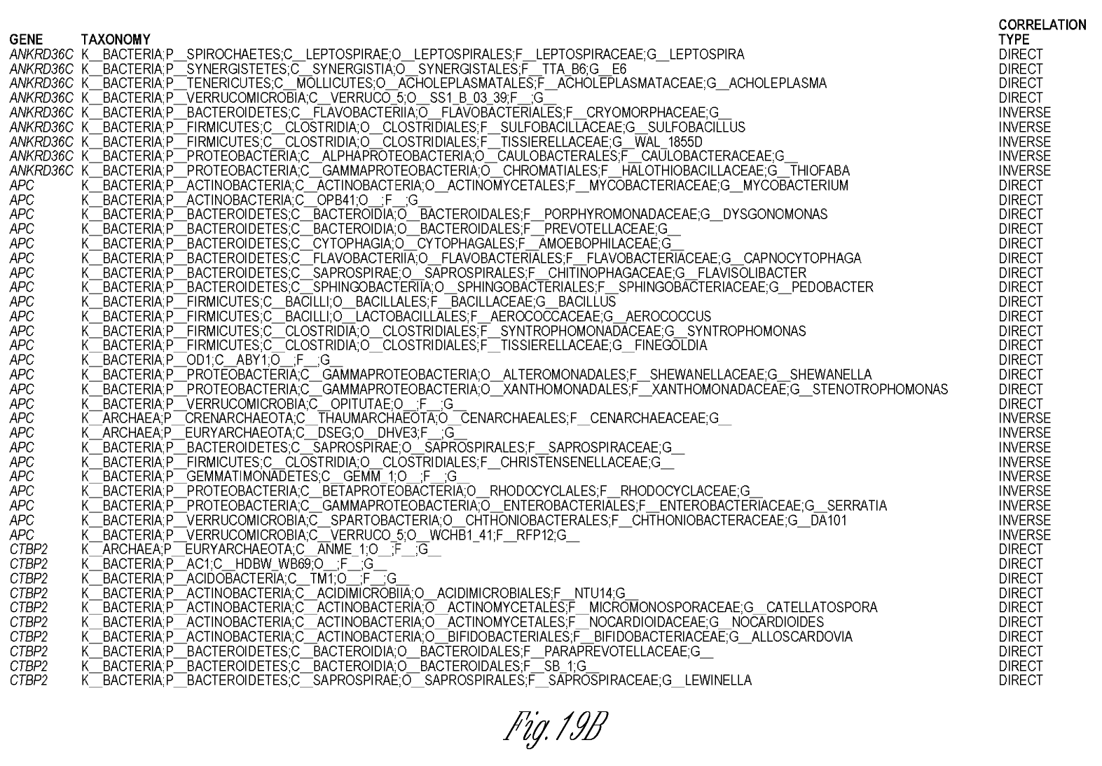

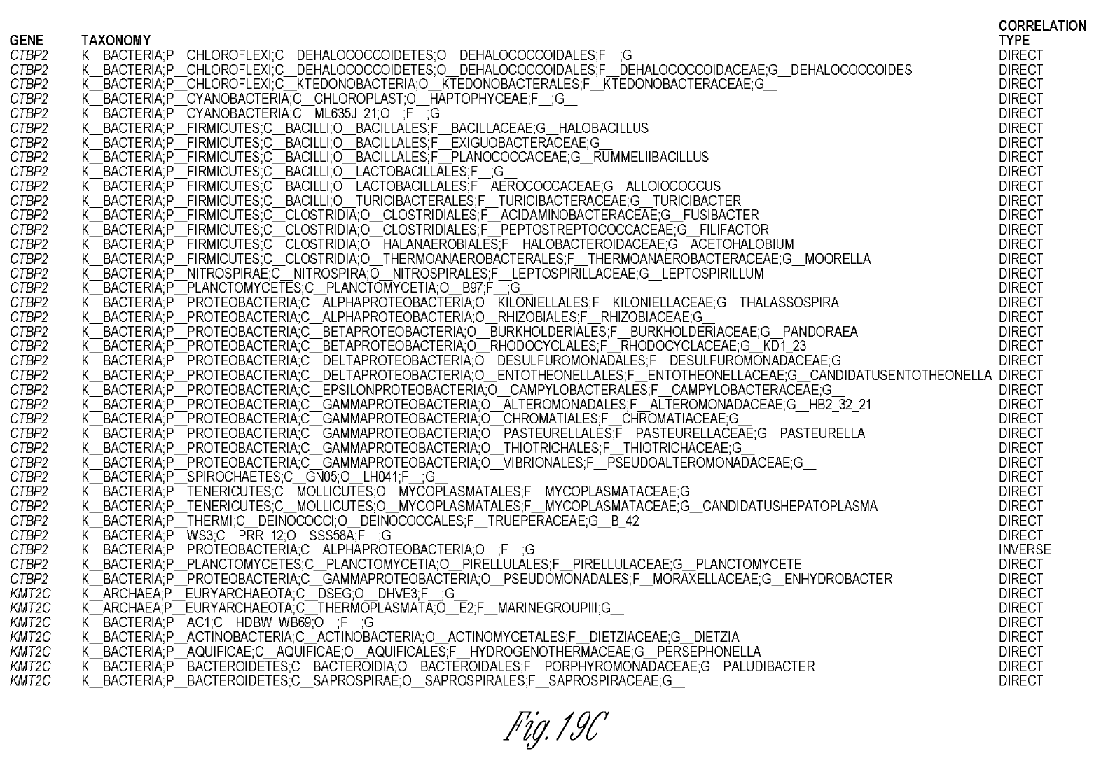

Next, a similar approach was used to classify tumors based on mutational profiles. The initial focus was on individual genes that harbor loss-of-function (LoF) mutations, as those, it was predicted, would be the most likely to have a physiologically relevant interaction with the surrounding microbiome. A prevalence filter was applied to include only those mutations that were present in at least 10 or more patients at the gene level. The raw OTU table was collapsed to the level of genus for the analysis. A visualization of the correlations between gene-level mutational status and the associated microbial abundances revealed differing patterns of abundances that suggests an interaction between the 11 most prevalent LoF tumor mutations and the microbiome (FIG. 9). It was hypothesized that the presence of mutation-specific patterns of microbial abundances could be statistically described by prediction of tumor LoF mutations in individual genes using the microbiome. For each of eleven genes that passed prevalence filtering cutoff, we identified the associated microbial taxa (FIG. 6A and FIGS. 18-19), generated risk indices for each patient (FIGS. 6B-C), and plotted the mean differences in abundances for a subset of microbial taxa interacting with each mutation (FIG. 6D). The microbiome composition profiles could be used to predict the existence of tumor LoF mutations in the human genes APC, ANKRD36C, CTBP2, KMT2C, and ZNF717 (Q-value=0.0011, 0.0011, 0.019, 0.019, and 0.055, respectively, by permutation test after False Discovery Rate (FDR) correction for multiple tests with a Q value threshold of 0.10; FIG. 6). The risk indices for each mutation were generated using sets of microbial taxa that ranges from 22 (ZNF717) to 53 (ANKRD36C) taxa (FIG. 19). The taxa that showed the most dramatic differences in abundance when comparing tumors with and without mutations are shown in FIG. 6D. For example, the abundance of Christensenellaceae is relatively lower in tumors with APC mutations, but relatively higher in tumors with ZNF717 mutations.

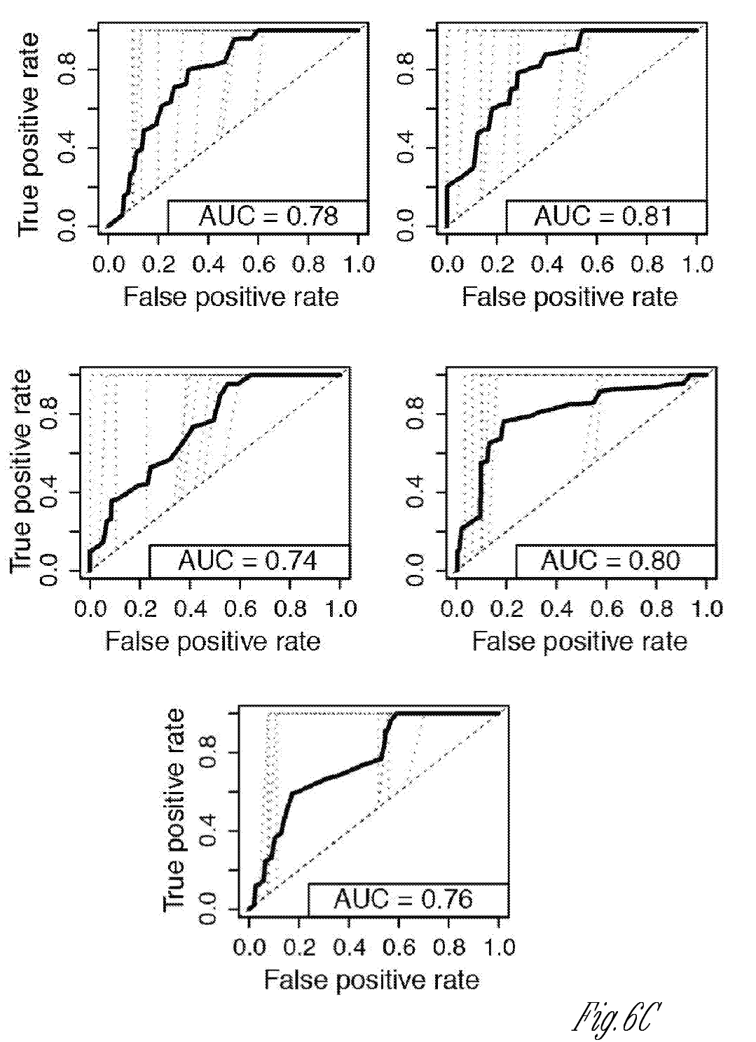

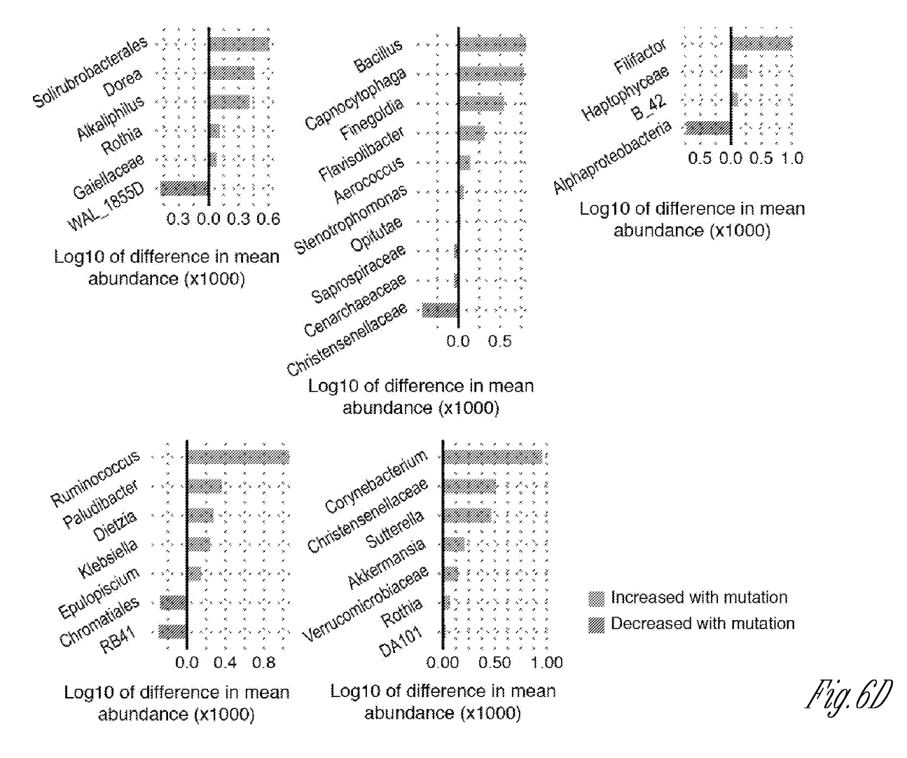

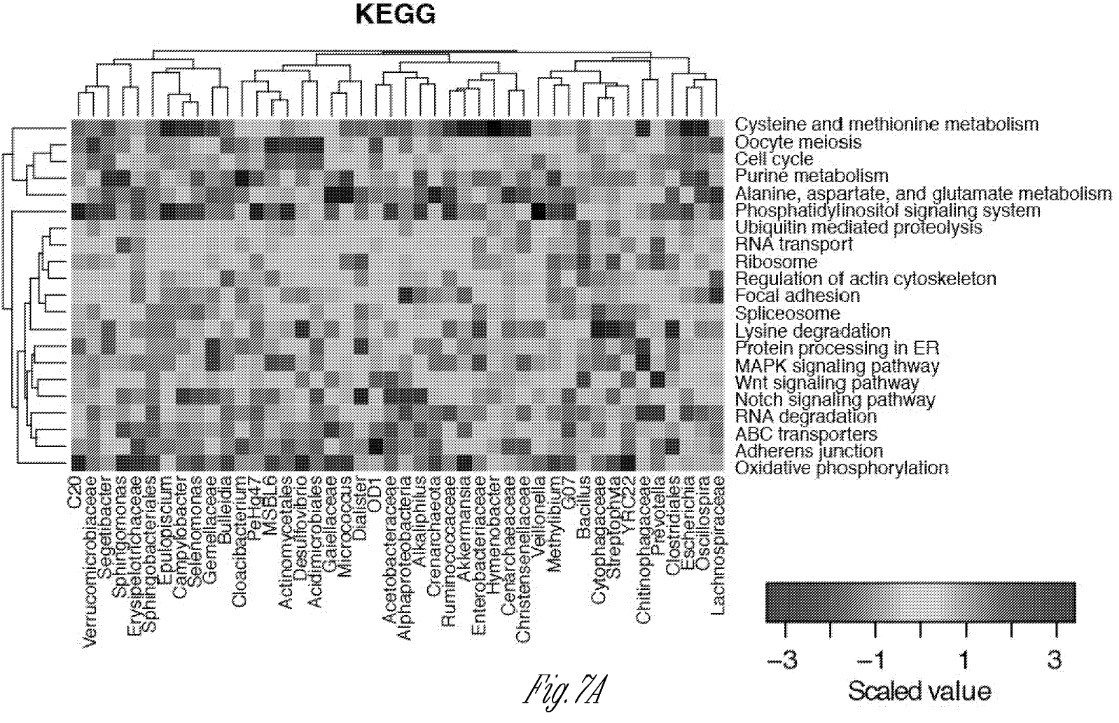

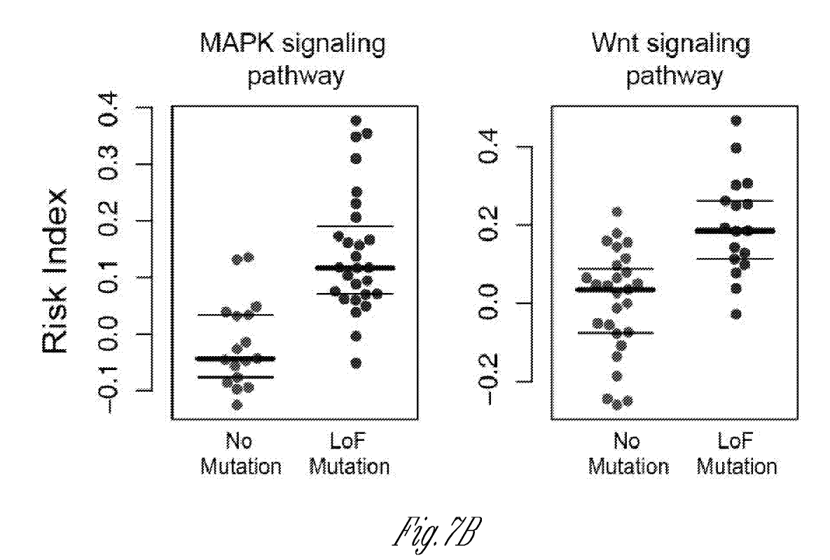

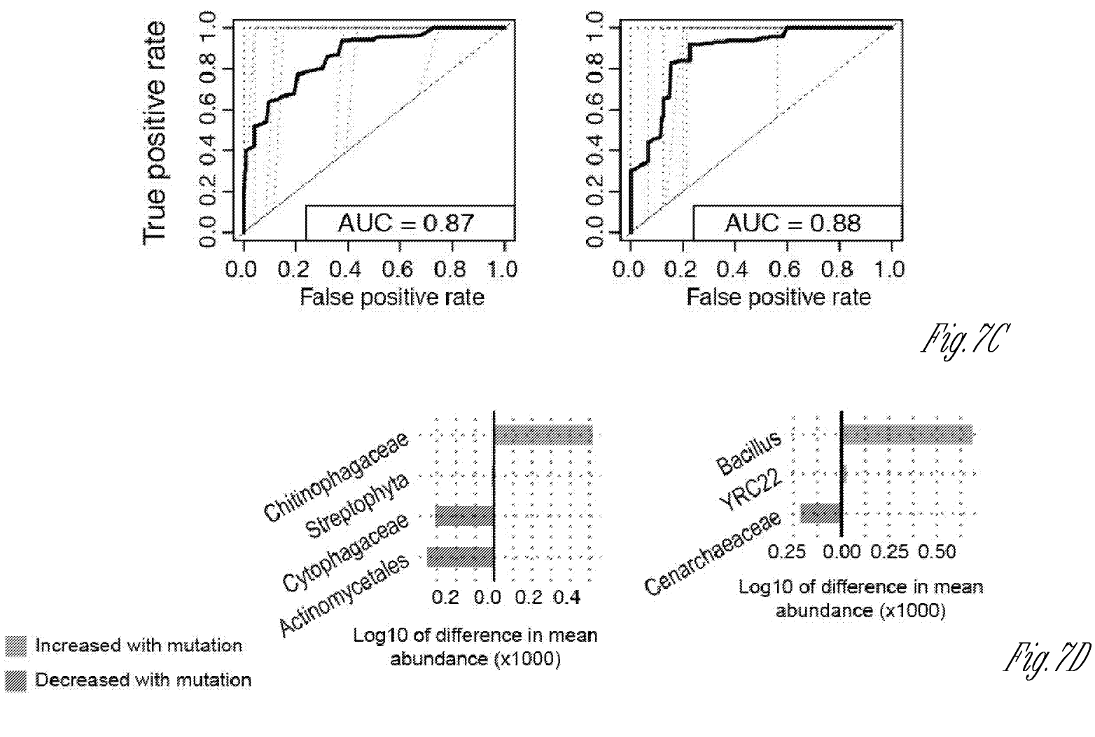

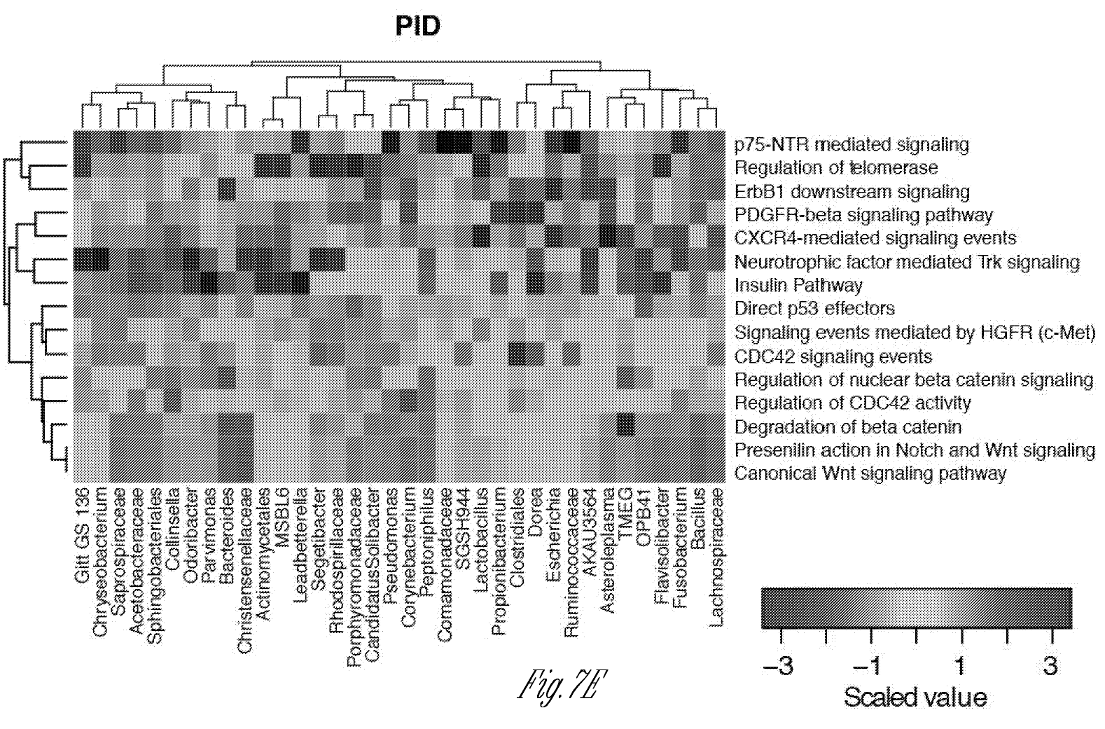

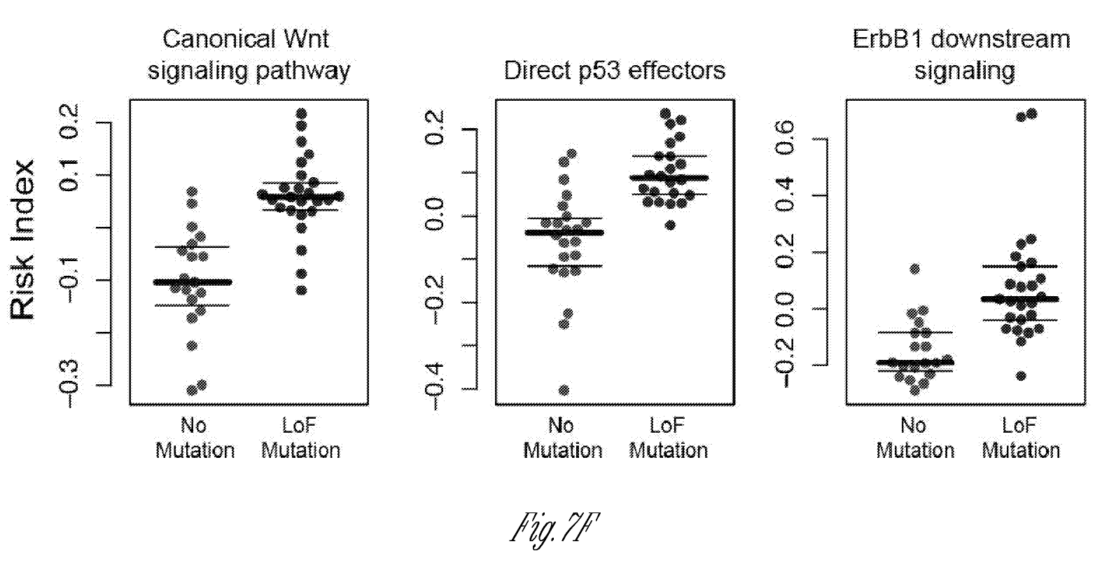

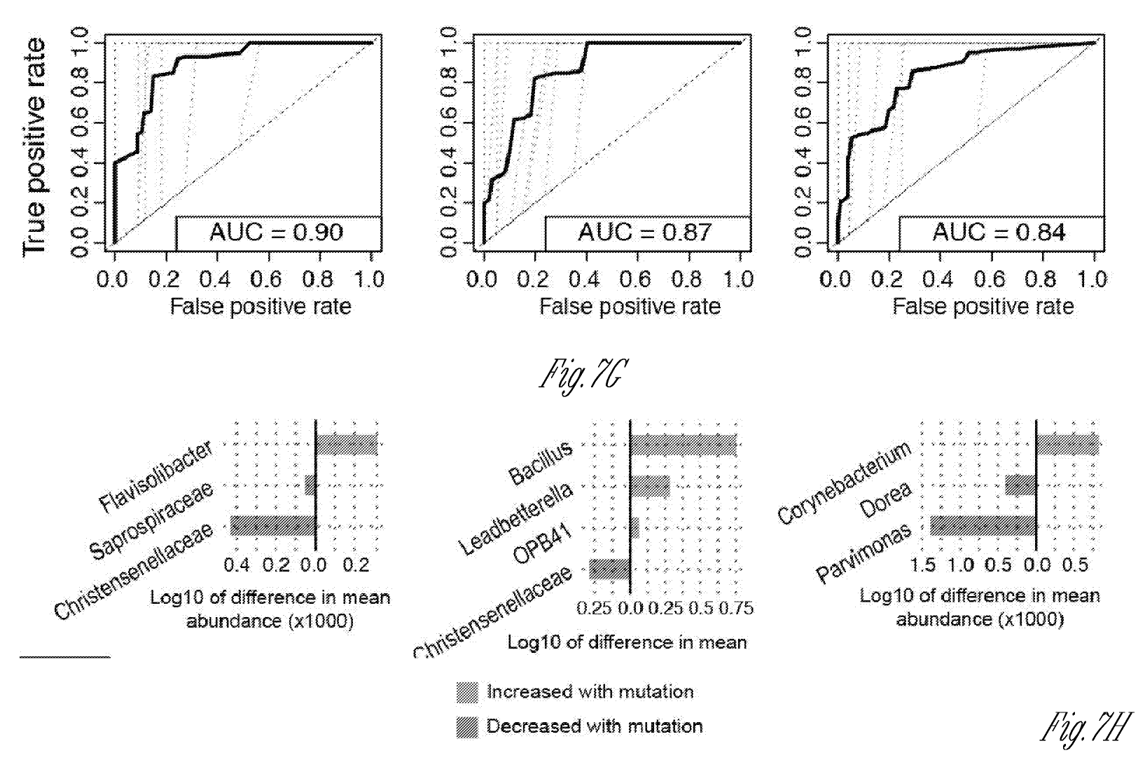

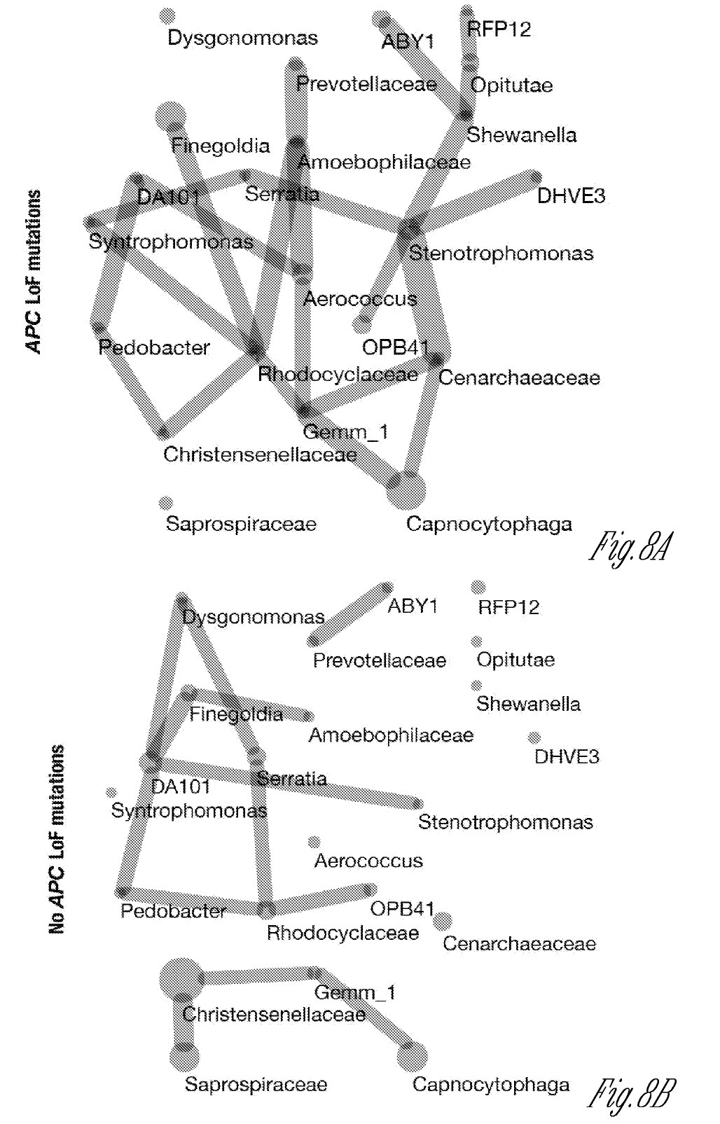

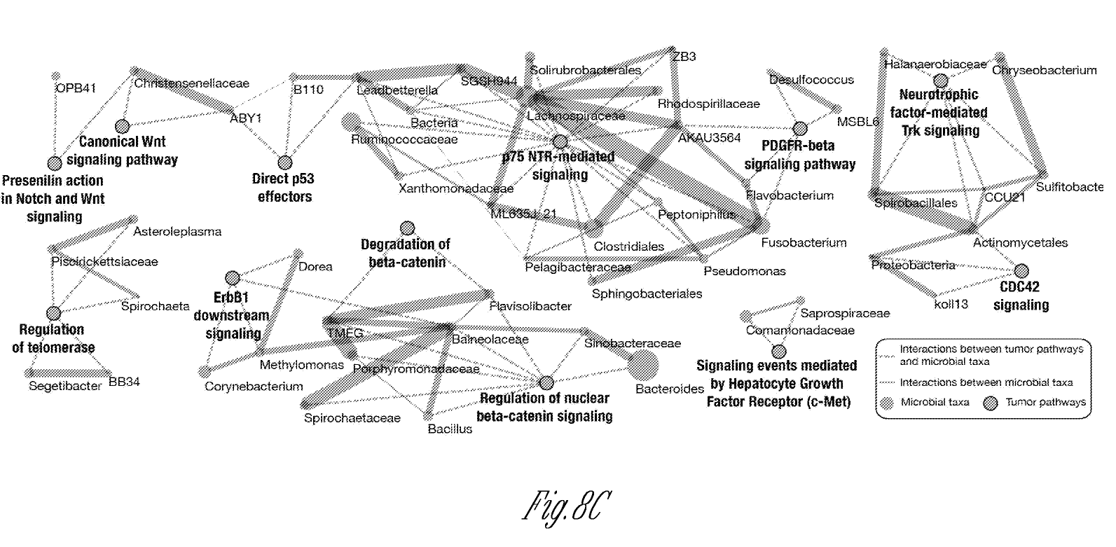

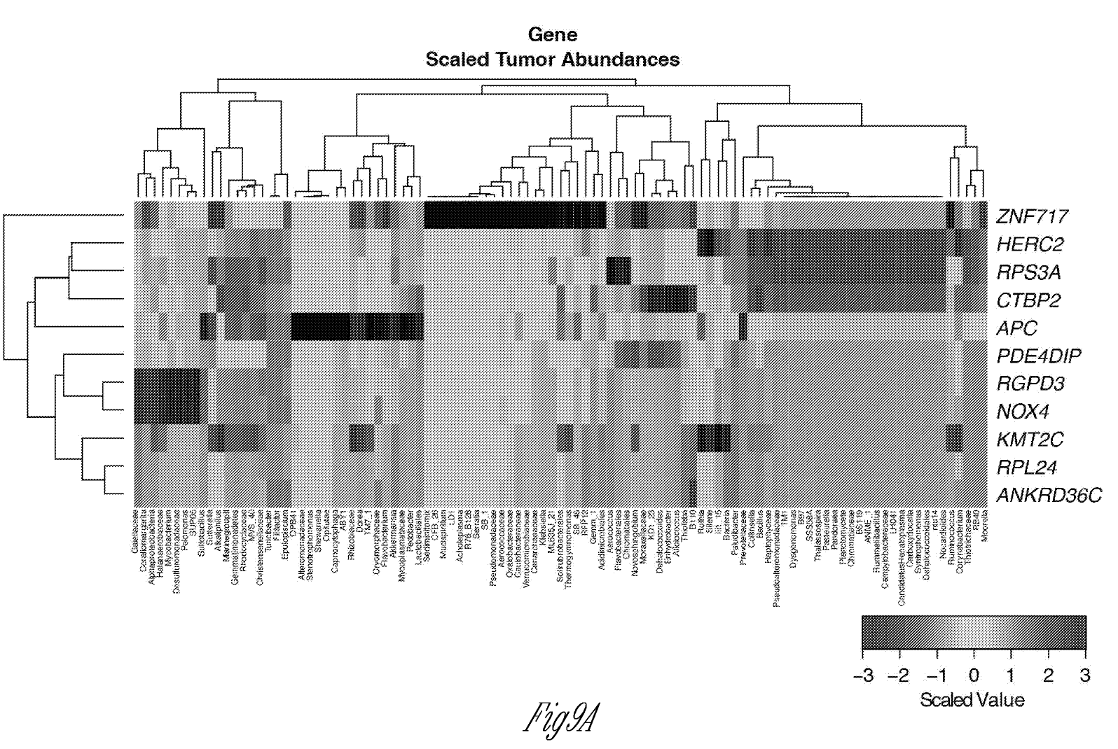

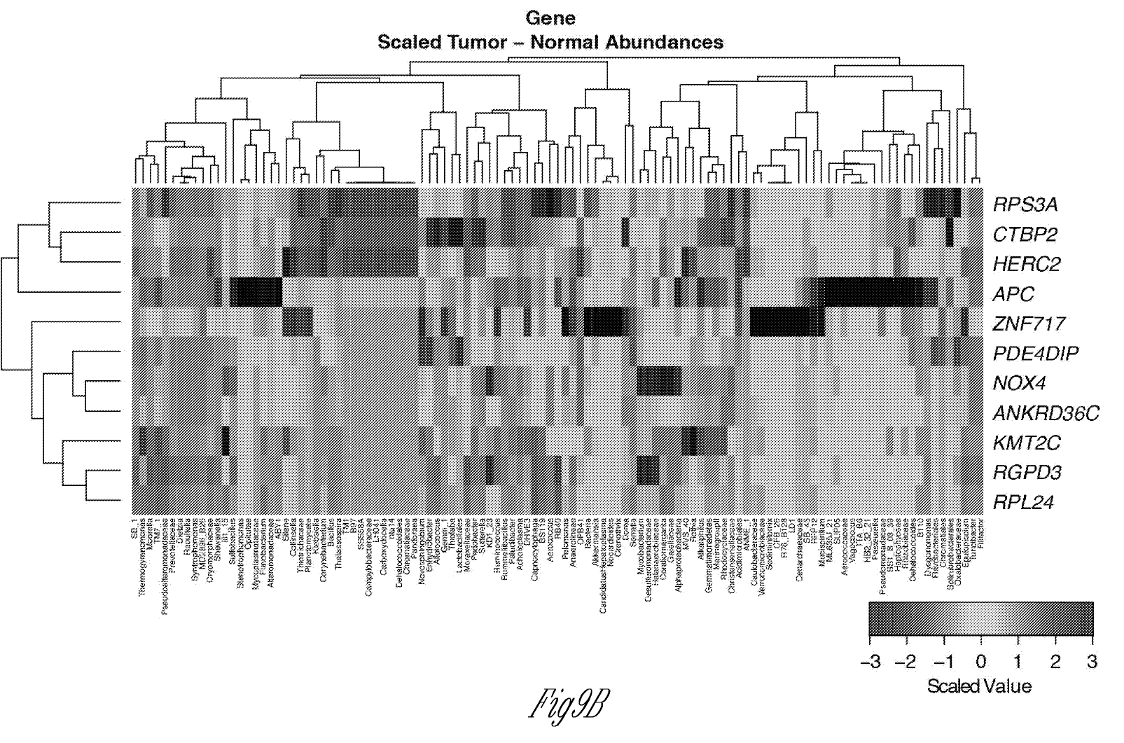

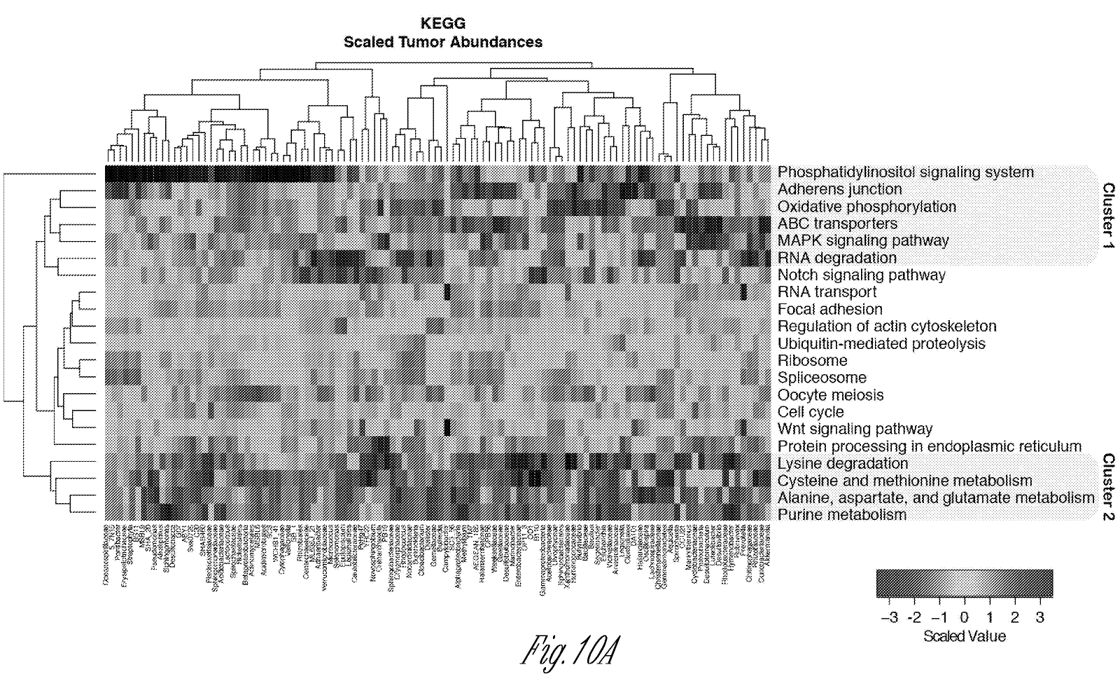

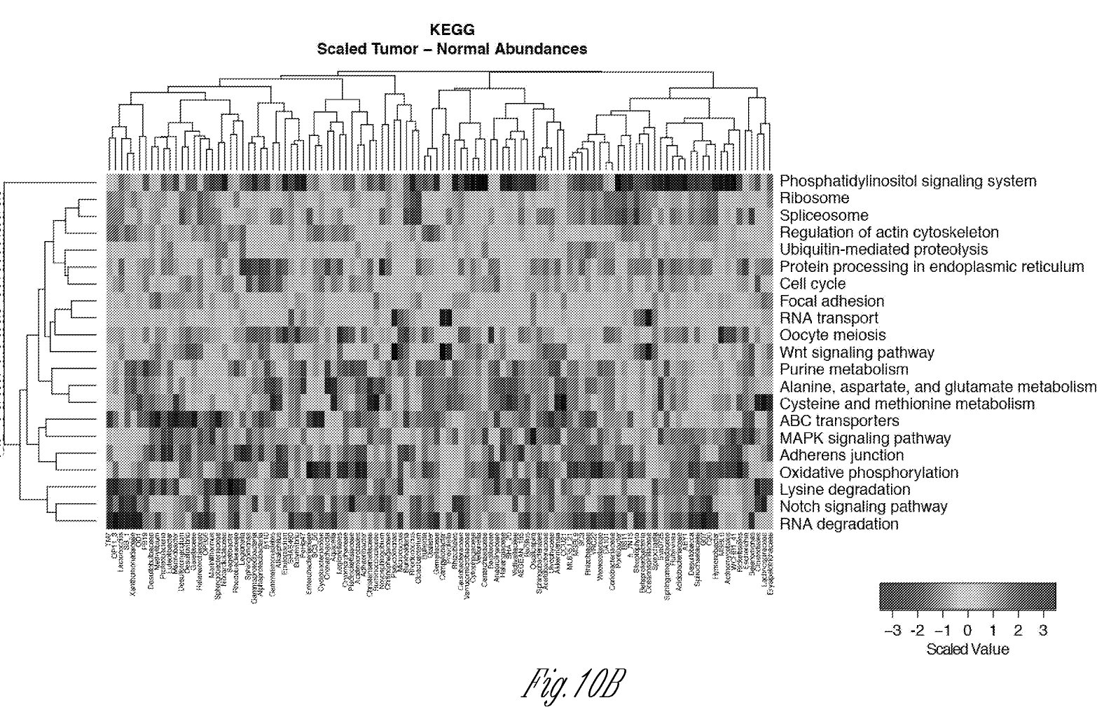

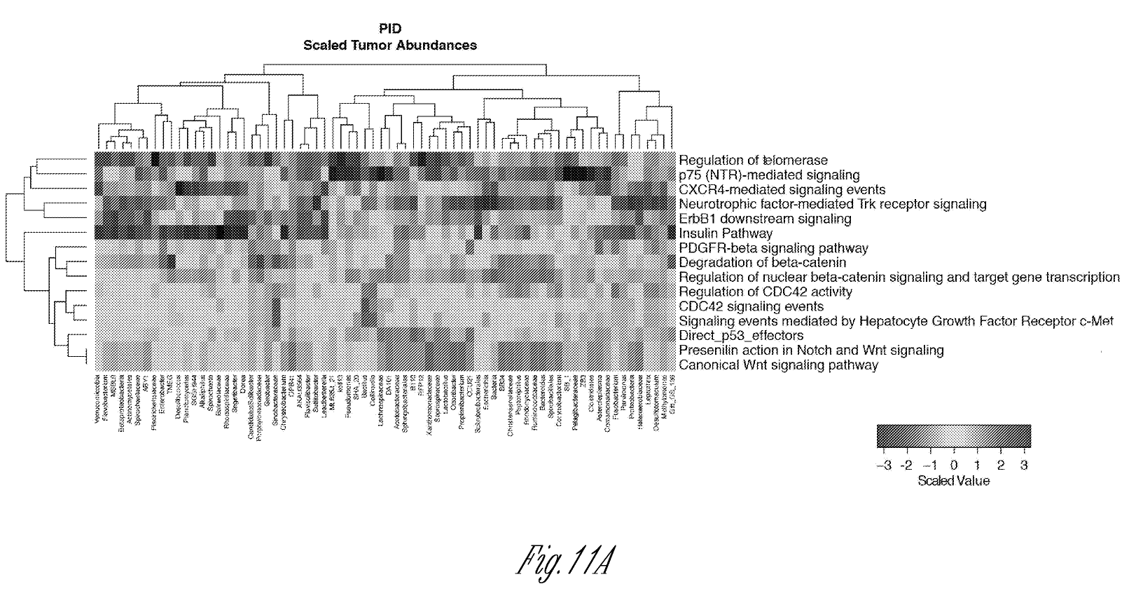

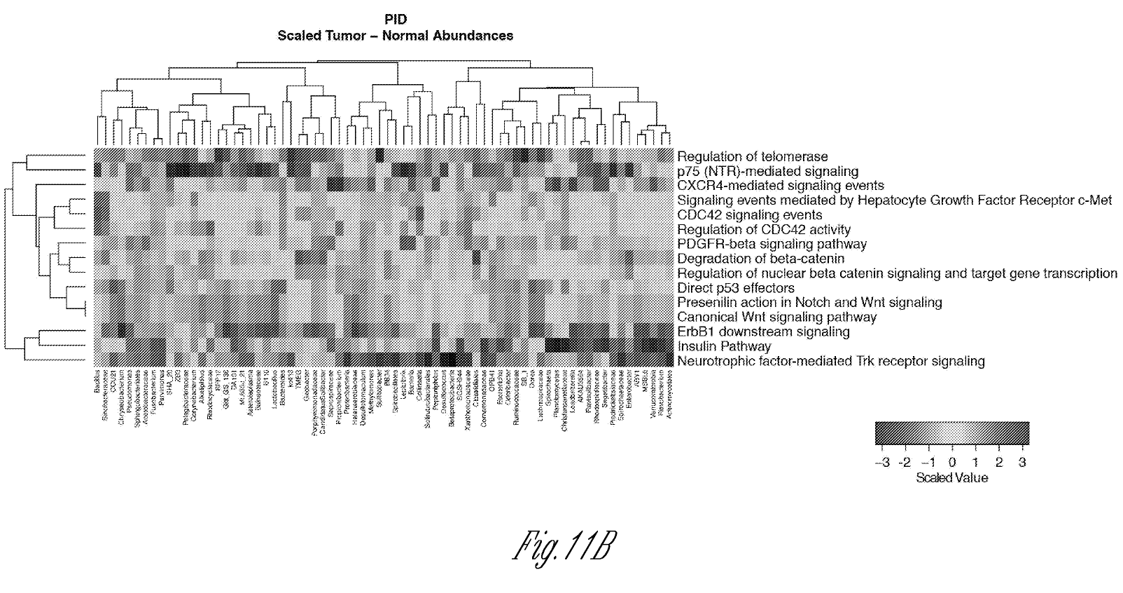

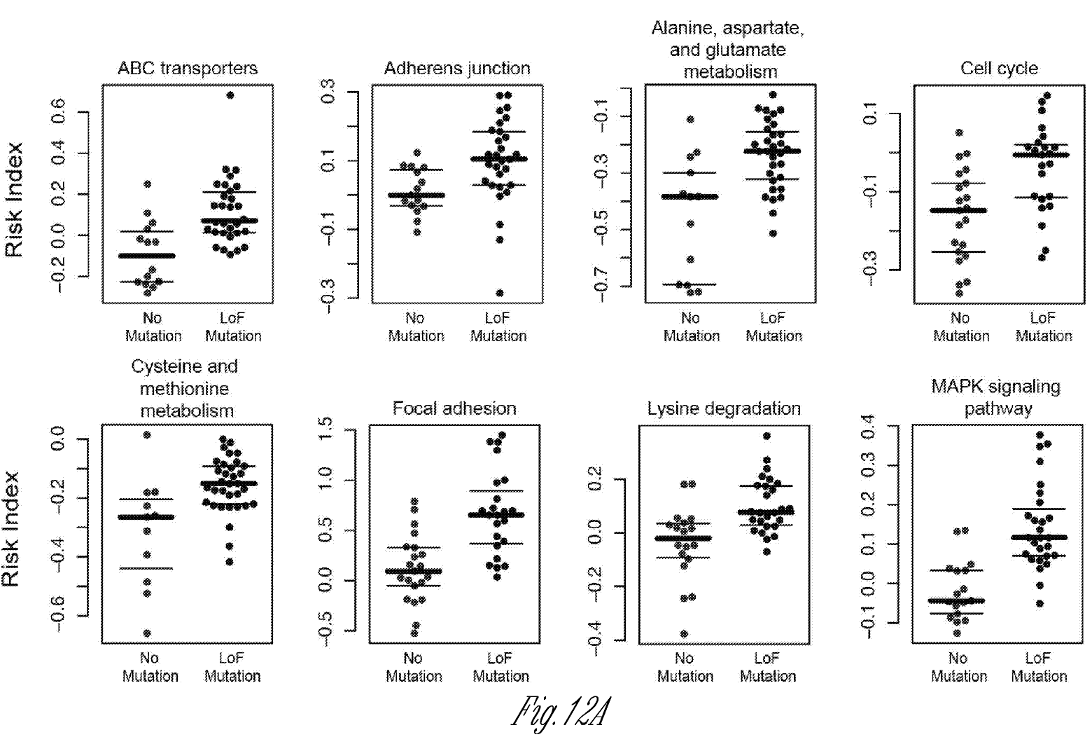

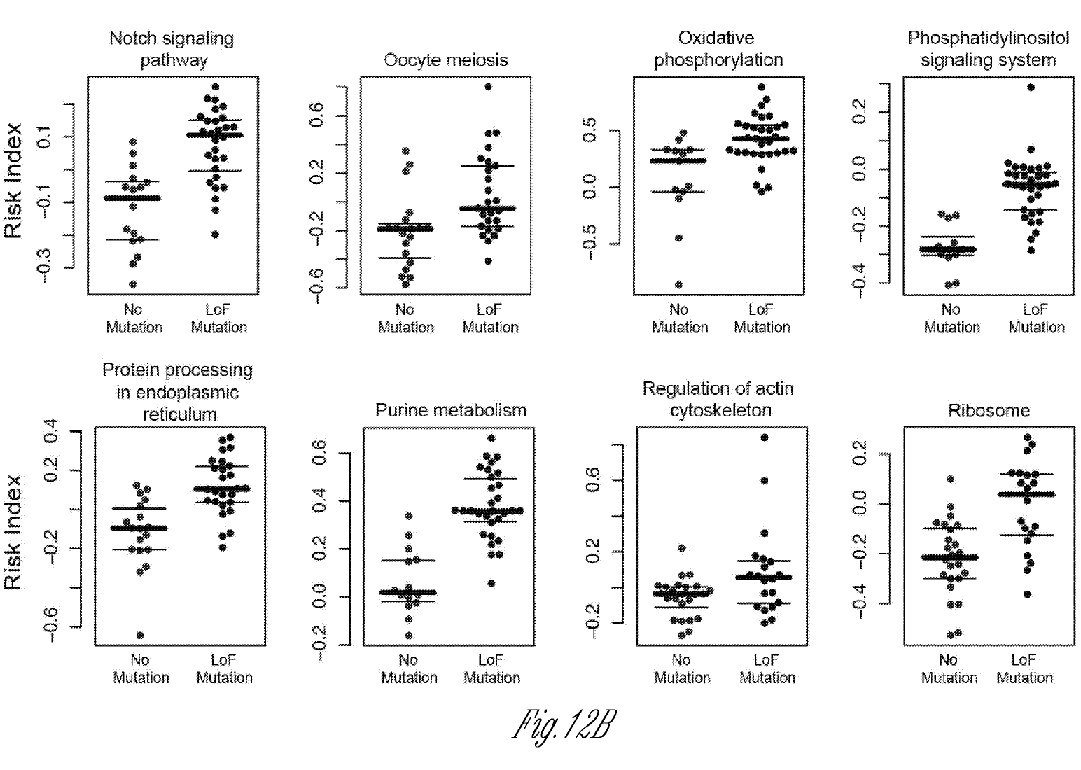

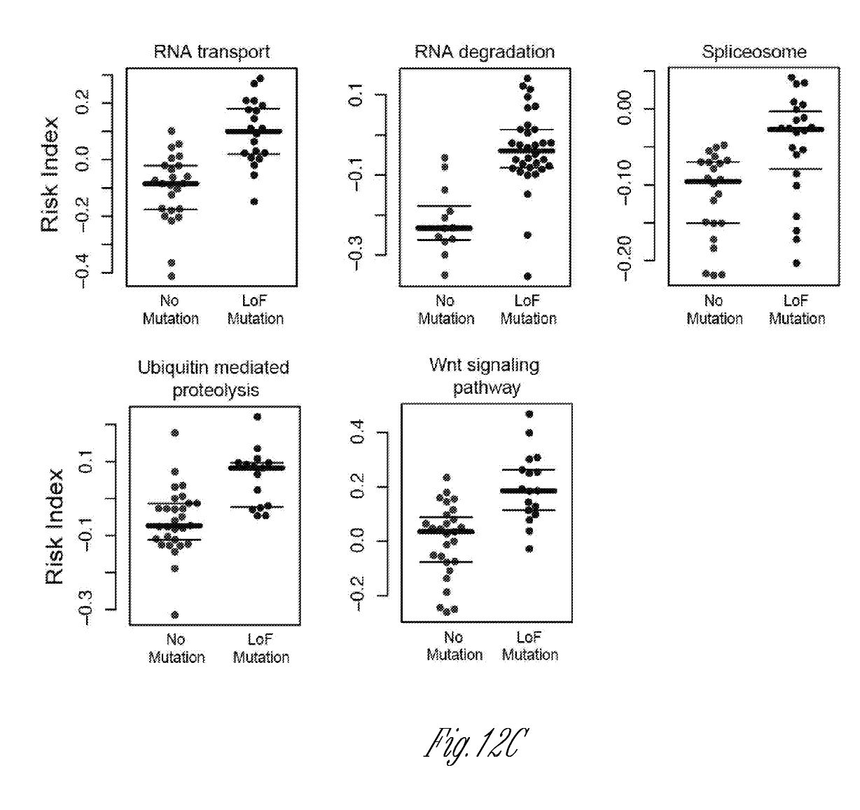

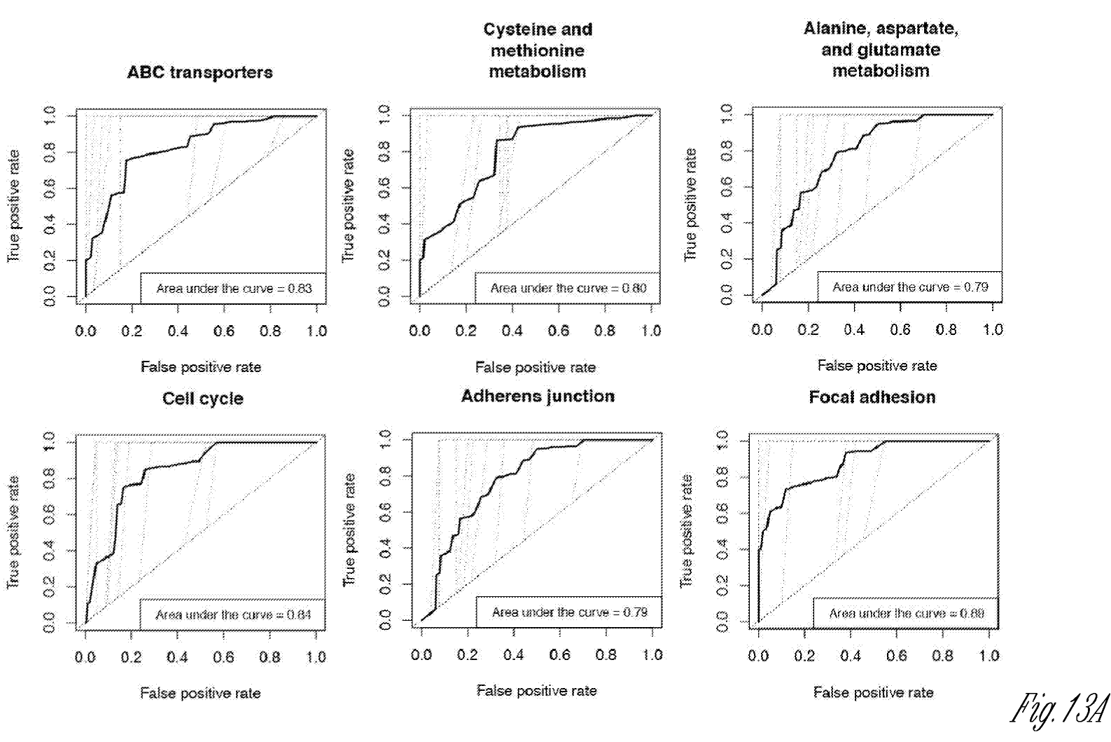

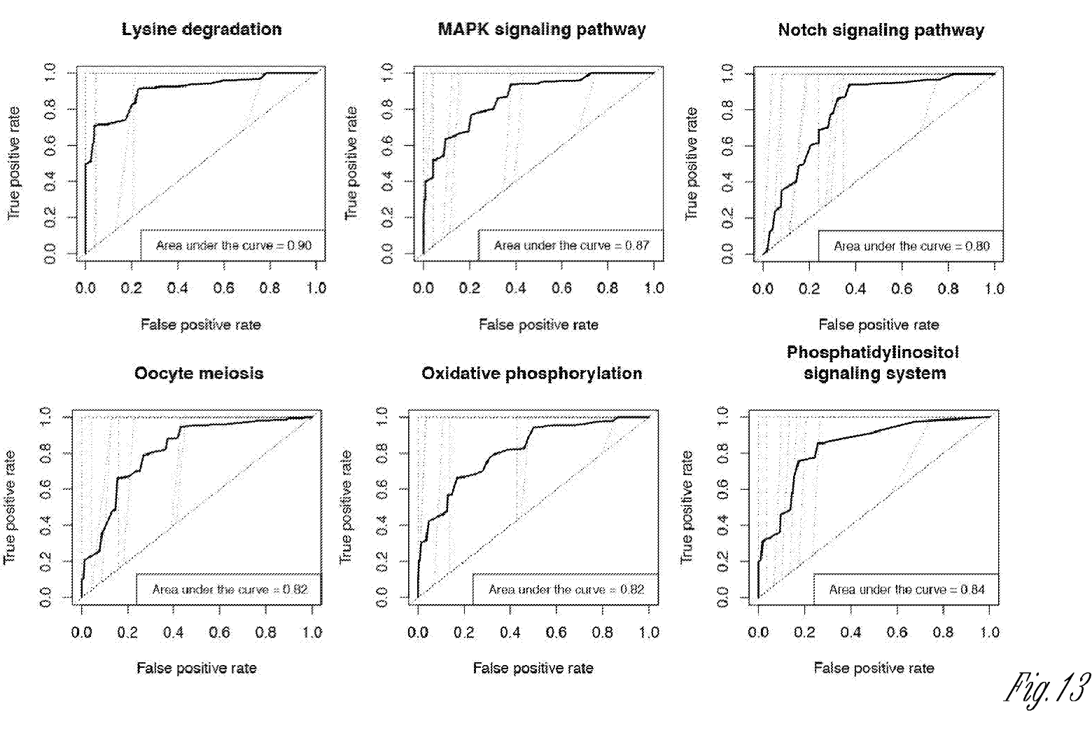

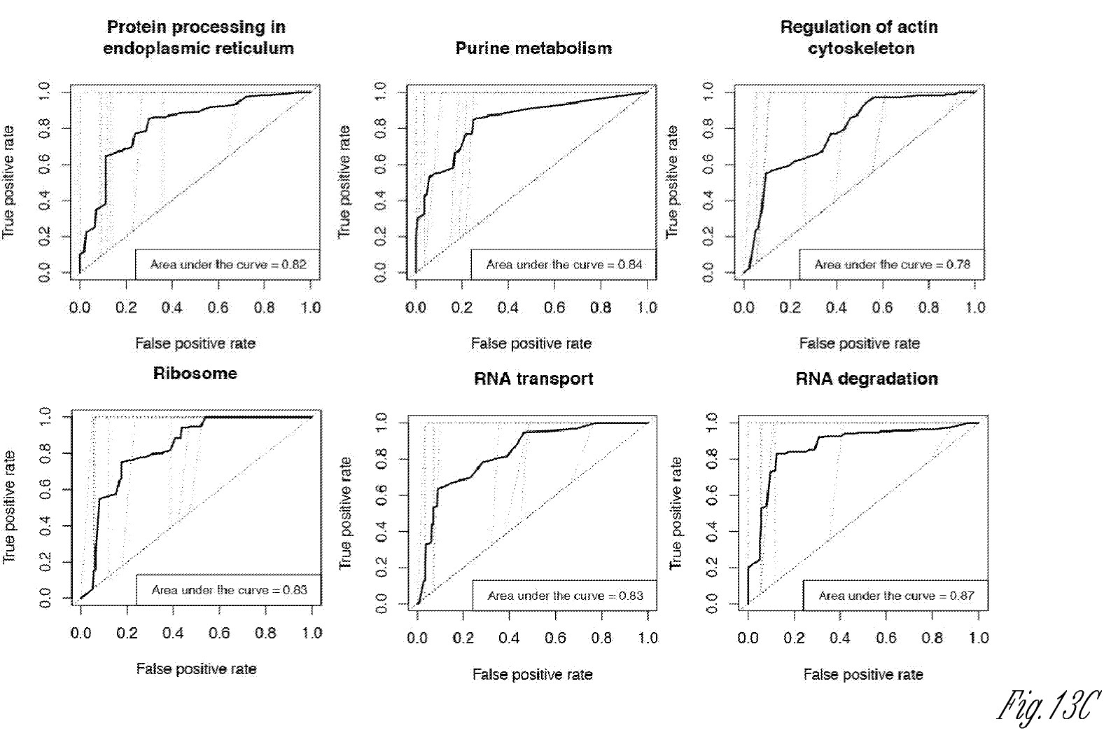

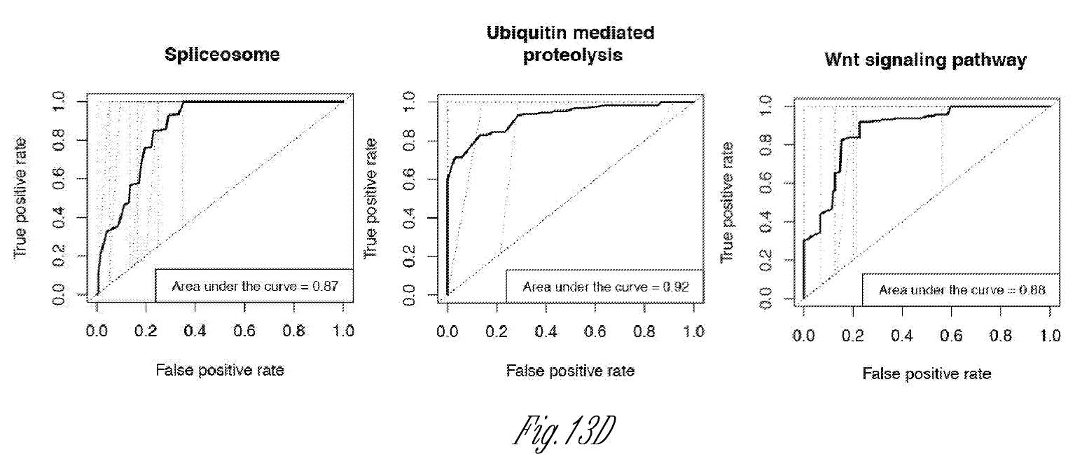

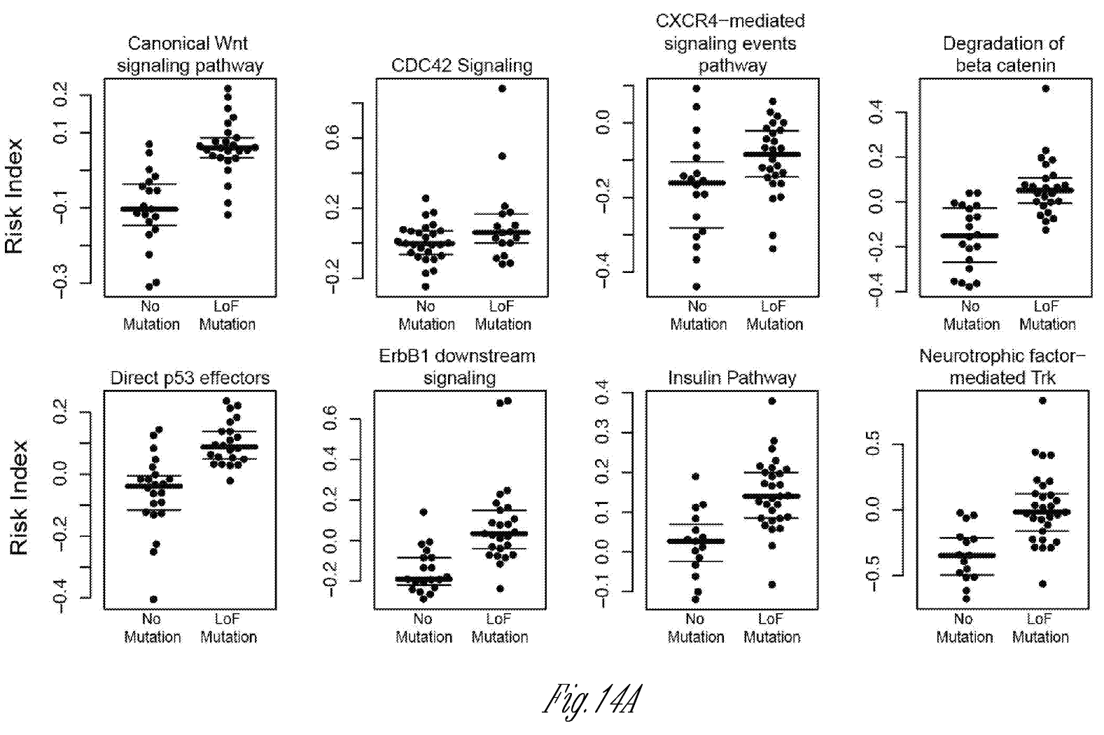

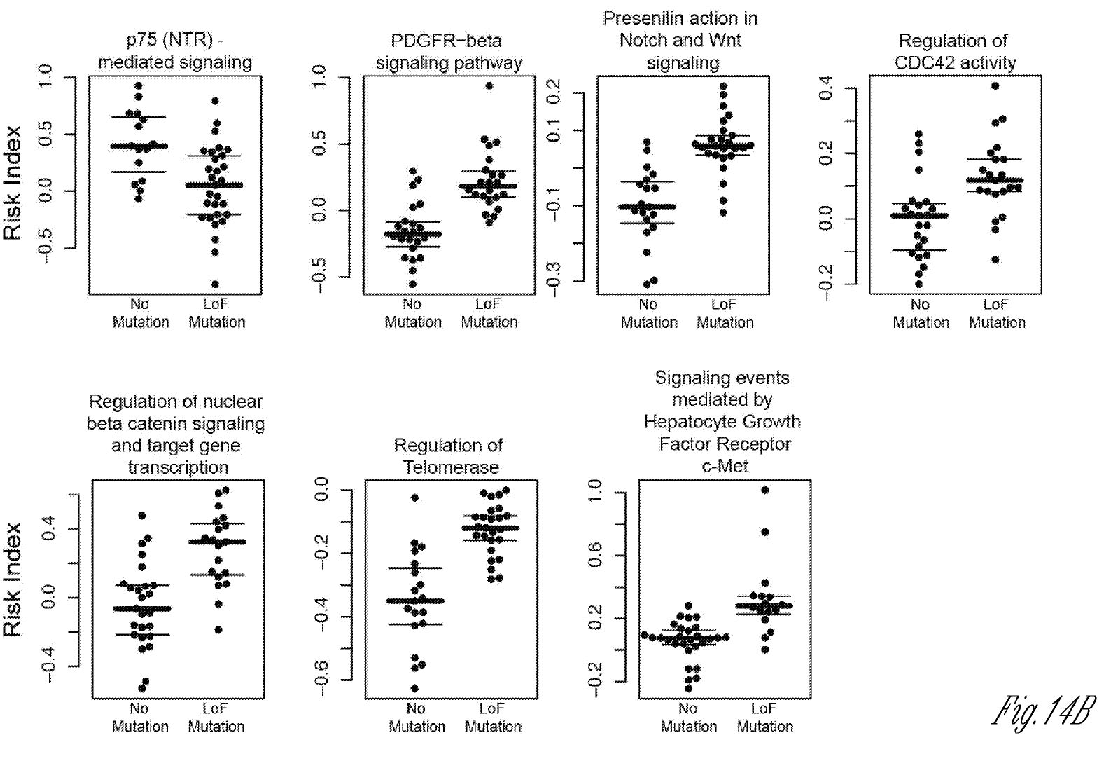

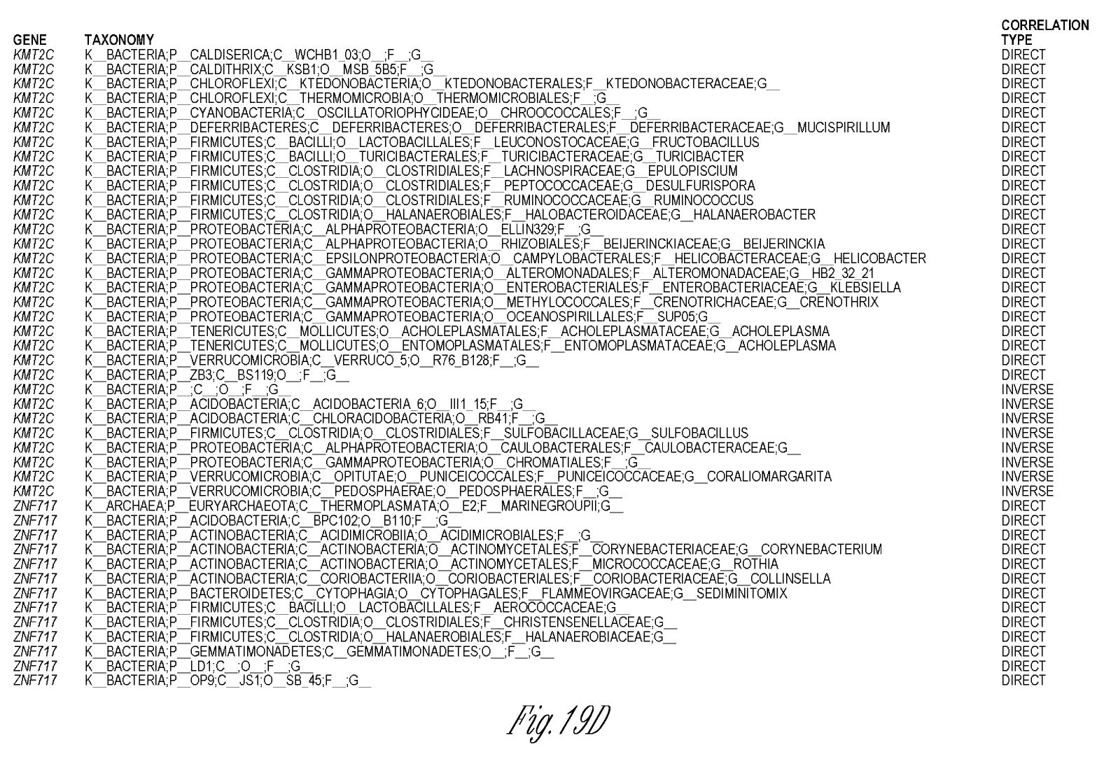

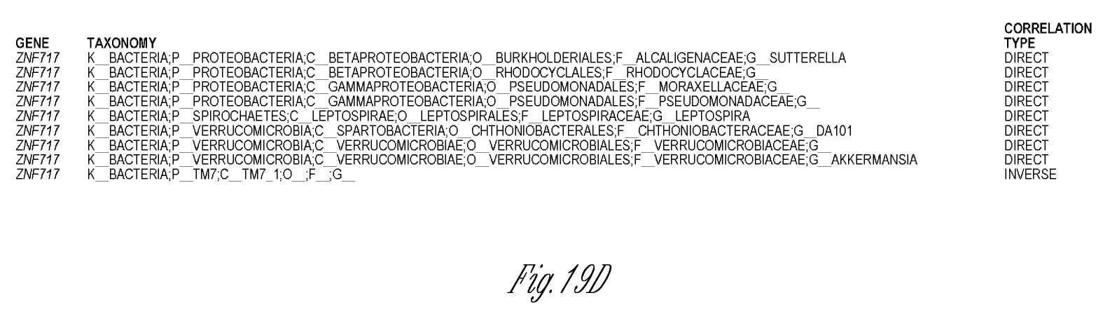

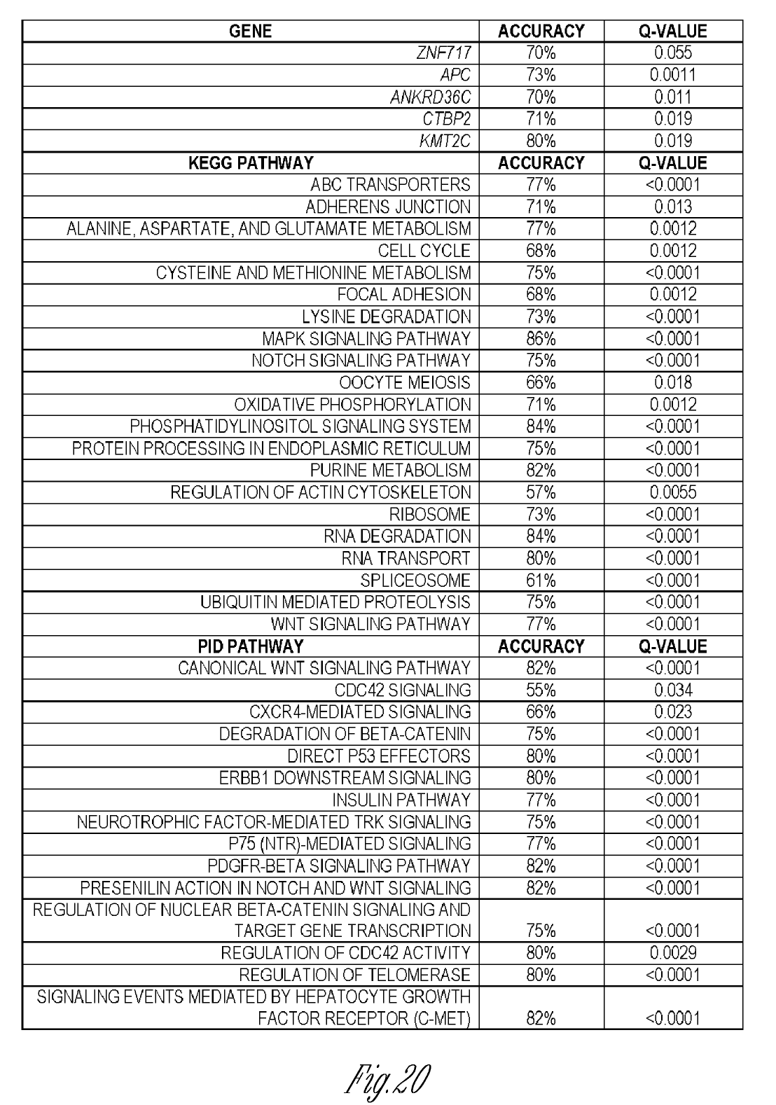

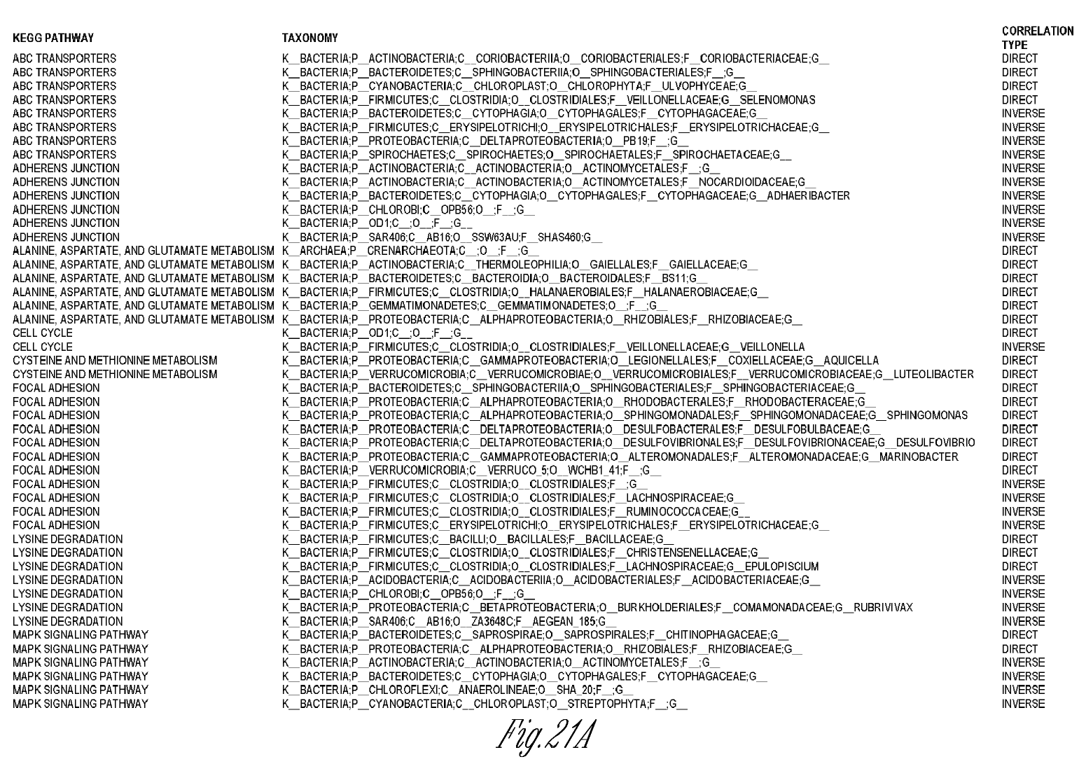

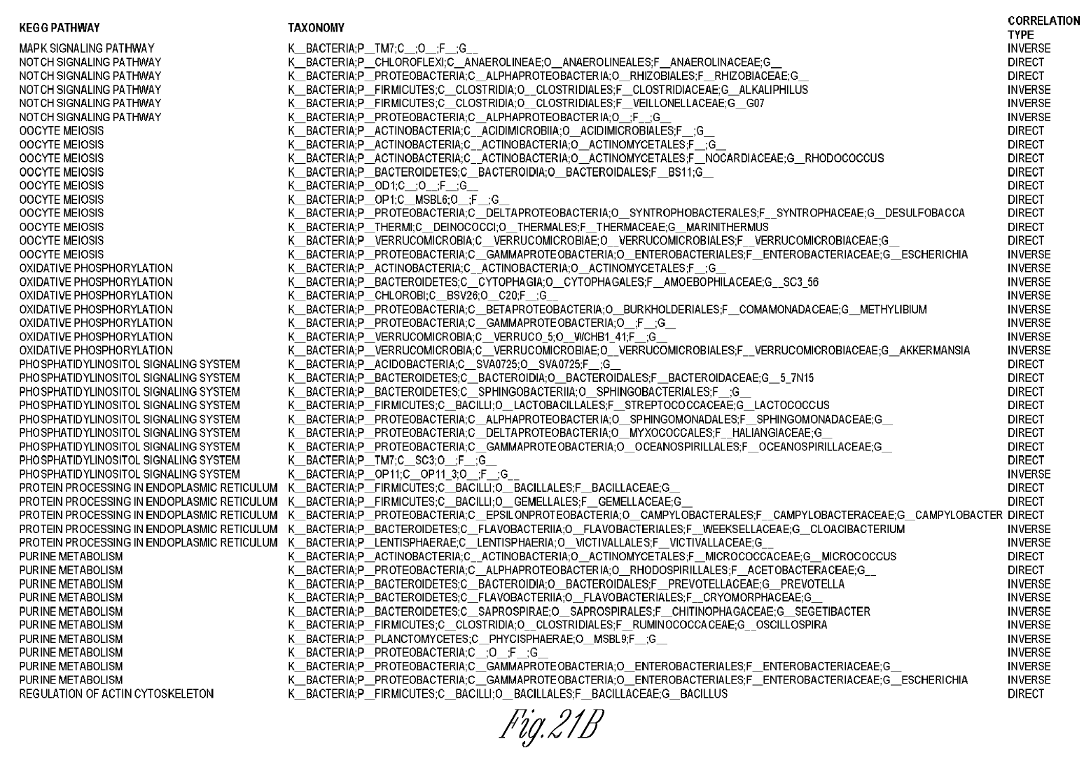

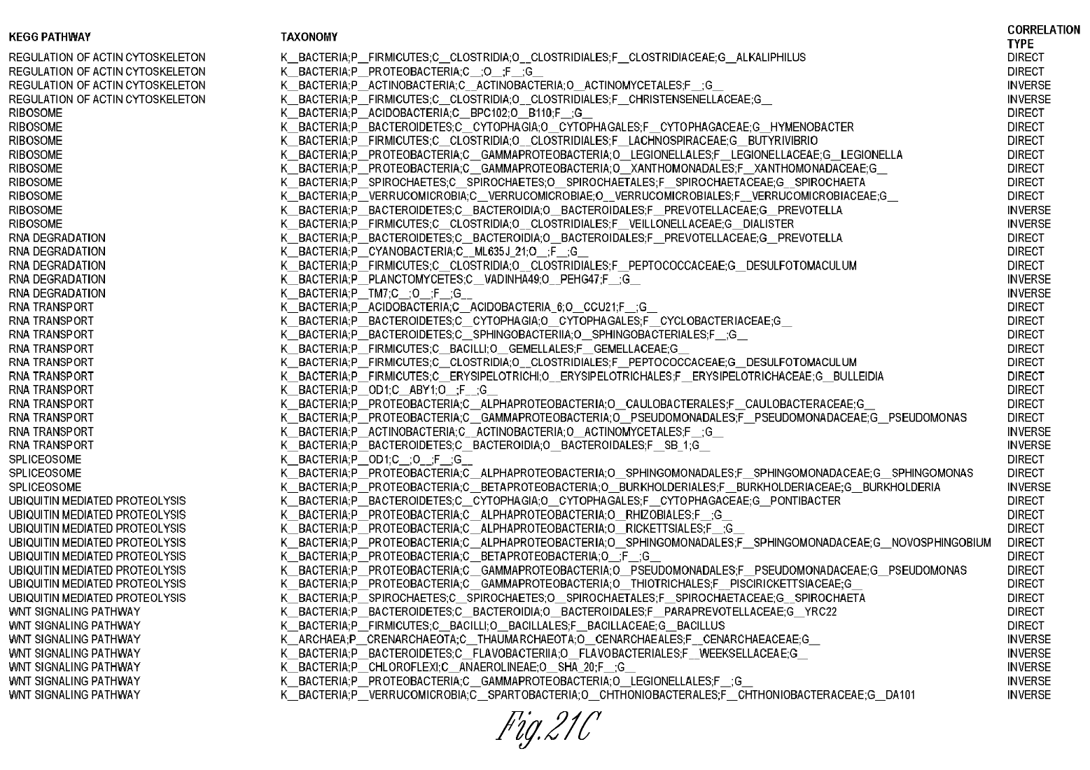

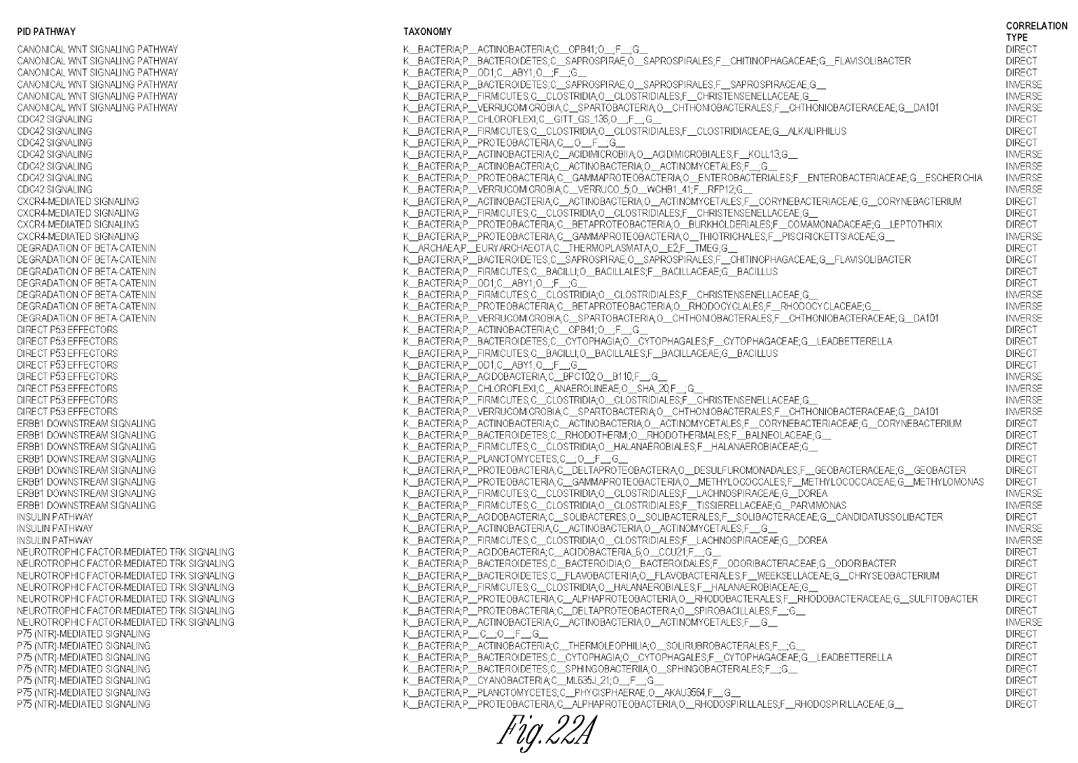

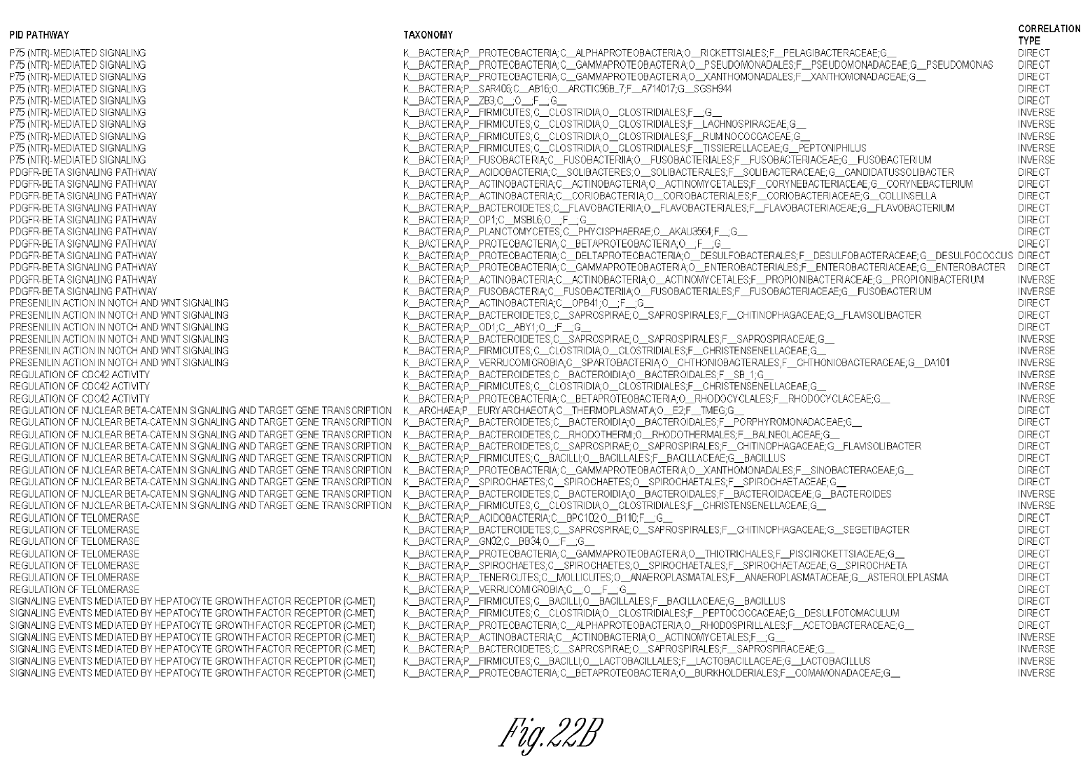

Next, the interaction prediction approach, as described above, was applied to the pathway-level mutational data. Following visualization of the pathway level abundances (FIGS. 10-11) and applying the model, it was found that each of the 21 KEGG pathways passing prevalence filter were able to be significantly predicted with a fixed specificity of 80% and an accuracy up to 86% (Q-values<0.02 by permutation test after FDR correction; FIGS. 7A-D, 11-12 and 20), as were 15 of the 19 tested PID pathways (Q-values<0.04 by permutation test after FDR correction) (FIGS. 7E-H, 14-15 and 20). The taxon abundances that were specifically associated (direct or inverse correlations) with each of the LoF mutations in the genes and pathways can be found in FIGS. 16, 21 and 22. In general, the number of taxa within each of the sets used to generate the risk indices was lower than that used for the gene-level analyses (average of 37 taxa per gene-associated set compared to 7 taxa per set associated with mutations in KEGG or PID pathways). When comparing results using the gene-level interactions and the pathway level interactions, for instance looking at mutations in APC (FIG. 6) and comparing them to mutations in the KEGG-defined Wnt signaling pathway and the PID-defined Canonical Wnt signaling pathway (FIG. 7), the interactions at the pathway level are more statistically significant (AUC for APC=0.81, KEGG=0.88, PID=0.90). This trend is consistent and can be visualized as a density histogram of interaction prediction accuracies.

### Predicted Microbiome Interaction Network Affected by Tumor Mutational Profile

Lastly, the correlations between taxa among tumors with and without LoF mutations was assesed (FIG. 8). Striking differences were found in the structure of the network comparing tumors with and without a Lof mutation in APC the correlations between taxa (FIG. 8A). For example, in tumors with mutations in APC, the abundance of Christensenellaceae is positively correlated with Rhodocyclaceae and negatively correlated with Pedobacter. In tumors lacking LoF mutations in APC, these correlations are lost and Christensenellaoeae is instead negatively correlated with Saprospiraceae and Gemm 1. The network of correlations across tumors with mutations in PID pathways was also assessed (FIG. 8B). This analysis highlighted that some pathway-level mutations show a shared set of correlations between taxa, while others appear independent. Several of the taxa that can be used to predict LoF mutations in p75(NTR) signaling share correlations among each other as well as with taxa associated with mutations in PDGFR-beta signaling and direct p53 effectors.

### DISCUSSION

The link between colorectal cancer and the gut microbiome has been highlighted by a large number of recent studies (Burns et al., 2015; Warren et al., 2013; Nakatsu et al., 2015; Wang et al., 2012; Shen et al., 2010; Chen et al., 2012; Zhu et al., 2014; Sing et al., 2014; Flemer et al., 2016; Wynedaele et al., 2015; Rubinstein et al., 2013; Zeller et al., 2014; Zackular et al., 2014; Yu et al., 2015; Baxter et al., 2016; Miora et al., 2014; Kostic at al., 2012), with several hypotheses as to the causal role of microbes in the disease (Song et al., 2014; Rubinstein et al., 2013; Dennis et al., 2013; Irrazabal et al., 2014). Since cancer is a genetic disease caused by mutations in host DNA, it is of interest to study the microbiome in the context of tumor mutational profiles, especially given recent studies showing an impact of host genetics on the microbiome (19-24). Here, tumor coding mutational profile and the taxonomic composition of the proximal microbiome were jointly analyzed. It was found that the composition of the proximal microbiome is correlated with mutations in tumor DNA, and that this correlation can be used to predict mutated genes and pathways solely based on the microbiome.

Quality control of the data and stringent filtering was conducted at every step (e.g., requiring 30× coverage at a site in both the tumor and matched normal sample to call a mutation). While these requirements are likely to increase the frequency of false negatives (true mutations that simply do not meet our criteria), this rigorous strategy is appropriate as a means of increasing the biological relevance of our findings. Of note, when comparing the common LoF mutations found in our dataset to those found in colorectal tumors sampled as part of The Cancer Genome Atlas (TCGA) project, we find several commonalities, including a high frequency of LoF mutations in APC, as well as numerous missense mutations in KRAS, NRAS, and TP53, as expected (Cancer Genome Atlas Network).

The association of microbial taxa with tumor stage (FIG. 5) mirrors recent results, including a study of a Chinese population (Nakatsu et al., 2015; Ahn et al., 2013). This concordance is relevant as it indicates that the microbial communities appear to be consistent even when comparing geographically distinct patient cohorts (Yatsunenko et al., 2012; Allali et al., 2015). One of the predictive taxa, Porphyromonadaceae, has been shown to be altered in mouse models of CRC in other studies as well (Chen et al., 2012; Zachular et al., 2014). A study on the link between dysbiosis and colitis-induced colorectal cancer also showed similar results (Couturier-Maillard et al., 2013). For instance, the bacterial genus Paludibacter was found to be associated with risk of developing tumors in a mouse model (Couturier-Maillard et al., 2013). Paludibacter was found to be significantly associated with low-stage tumors, again, supporting the hypothesis that these bacteria as associated with cancer risk and may be contributing to early stage inflammation (Couturier-Maillard et al., 2013). Conversely, it was found that the genus Coprococcus is associated with high-stage tumors and not low stage tumors. Members of this genus are known to generate butyrate and propionate, which in this context can act as antiinflammatory short chain fatty acids (Louis et al., 2014). Although the present results are correlational and cannot point to causal effects, these findings suggest that driving inflammation may play a role in early stage cancer, while generating nutrients at the cost of suppressing inflammation may be more beneficial to the tumor in later stages.

Gene-level mutation data, visualized in FIG. 9, show intriguing patterns of microbial abundances that are associated with the tumors harboring different mutations. For instance, as reflected in the differing patterns within each gene (rows) in the heatmap, Aerococcus and Dorea are both show higher scaled abundances within tumors harboring mutations in ZNF717, CTBP2, and APC, relative to tumors with LoF mutations in ANKRD36C and KMT2C. This highlights the different patterns in the microbiome that can be found when assessing genetically heterogeneous sets of tumors; as Dorea has been found to be increased in tumor microbiomes by several different groups, whereas the present work highlights some potential genetic interactions that explain cases wherein Dorea is not increased at the tumor site (Warren et al., 2013; Wang et al., 2012; Shen et al., 2010; Chen et al., 2012; Zhu et al., 2014). Thus, incorporating genetic profiles in studies of the microbiome in CRC may be beneficial and uncover patterns that are dependent on specific tumor mutations.

Although it may be difficult to ascertain the biological mechanism behind the predicted interactions among mutated genes and microbial taxa (shown in FIG. 6), it is possible to generate hypotheses based on what is already known in the relevant literature. For example, ANKRD36C encodes a protein that may have a role in ion transport in epithelial cells (Kumar et al., 1995). Additionally, we find that LoF mutations in APC correlate with changes in 25 different microbial taxa, including an increase in the abundance of the genus Finegoldia. This genus has been identified in previous studies of colon adenomas and also harbors species that act as opportunistic pathogens at sites where the epithelium has been damaged (Shen et al., 2010; Dowd et al., 2008; Murphy et al., 2014). In addition, Capnocytophaga has been identified as a potential biomarker for lung cancer (Yan et al., 2015). Interestingly, changes in the abundance of Christensenellaceae are predictive of mutations in both APC and ZNF717. A recent study in twins has identified Christensenellaceae as a taxon that is highly driven by host genetics (Goodrich et al., 2014). It was found that mutations in ZNF717, a transcription factor commonly altered in gastric, hepatocellular, and cervical cancers (Cui et al., 2015; Chen et al., 2013; Lando et al., 2015), are associated with Verrucomicrobiaceae and Akkermansia, which are both known to increase in conjunction with colitis (Berry et al., 2012). Alphaproteobacteria are significant contributors to our ability to predict mutations in CTBP2, a repressor of transcription known to interact with the ARF tumor suppressor (Paliwal et al., 2007). Changes in this bacterial taxon's abundance has also been found to be associated with prostate cancer, however a mechanism of action was not explored (Yu et al., 2015). It was also shown that mutations in KMT2C, a gene commonly co-mutated along with KRAS, could be predicted, in part, using the abundance of Ruminococcus (Fang et al., 2016). These bacteria have been previously implicated in inflammatory bowel disorders and colorectal cancer by multiple groups (Zhu et al., 2014; Hu et al., 2016; Tsuruya et al., 2016; Zhang et al., 2015).

Similar results were also evident when aggregating the mutations into KEGG and PID pathways (FIGS. 7 and 10-11) (Kanehisa et al., 2000, 2014, 2015). As an example, it was found that the abundance of microbes that predict KEGG pathways form two distinct clusters, and that the genus Escherichia has a higher scaled abundance in tumors with mutations in the KEGG pathways in cluster 1 relative to those in cluster 2 (FIG. 10). Cluster 1 contains adherens junctions, which are partially responsible for maintaining the intestinal barrier and interestingly, a disruption of the intestinal barrier in mice using cyclophosphamide was shown to cause a loss of adherens junction function and a concomitant increase in bacterial translocation into the intestinal tissue, including species of Escherichia (Yang et al., 2013). When examining the heatmap with LoF mutation collapsed into PID pathways (FIG. 11), differences were found in scaled microbial abundances between the tumors as a function of which pathways are mutated. For instance, we find lower abundance of Pseudomonas in tumors with LoF mutations in the pathways ‘regulation of nuclear β-catenin signaling and target gene transcription’, ‘degradation of β-catenin’, ‘presenilin action in Notch and Wnt signaling’, and ‘canonical Wnt signaling pathways’. Recent studies have shown that Pseudomonas strains that express the LecB gene can lead to degradation of β-catenin, providing hypothetical support for the concept that this genus may play a somewhat protective role in CRC by suppressing the Wnt signaling pathway (Cott et al., 2016). The mechanism that might explain this phenomenon is still unclear but may have to do with alterations in appropriate cell surface adhesion molecules for the LecB protein or a change in the content of the cellular microenvironment (Cott at al., 2016; Garber et al., 1987).

Many of the interactions identified here between bacterial taxa and mutations in PID pathways have been demonstrated experimentally in the literature. For example, in human oral cancer cells, it was shown that bacteria of interest were able to activate EGFR through the generation of hydrogen peroxide (55). In addition, the correlation between ErbB1 downstream signaling and increase in the abundance of Corynebacterium has been demonstrated mechanistically in a model of atopic dermatitis, whereby EGFR inhibition results in dysbiosis (the appearance of Corynebacterium species) and inflammation (Kobayashi et al., 2015). Specific depletion of Corynebacterium ablates the inflammatory response (Kobayashi et al., 2015). Moreover, our finding that the abundance of Fusobacterium is depleted in tumors with LoF mutations in the PDGFR-beta pathway, which may be explained by the dependence of several pathogenic strains of bacteria for functionally intact PDGFR signaling for adherence to intestinal epithelium (Manthex et al., 2014). In addition, p75(NTR) signaling has been shown to operate as a tumor suppressor by mediating apoptosis in response to hypoxic conditions and reactive oxygen species (Pflug at al., 1992; Kraemer et al., 2014; Wang et al., 2015; Le Moan at al., 2011). Alterations in this pathway have also been shown to be useful as a biomarker for esophageal cancer (Yamaguchi et al., Yamaguchi et al. 2016).

The present study has several caveats. First, the study only shows correlations, and cannot directly assess causal effects. Thus, it is not known whether the microbiome is altered before or after the appearance of specific mutations. Nevertheless, many of the predicted interactions described above have been previously tested, albeit across a wide variety of experimental systems and disease states, typically in isolation, for biological relevance and mechanism of action. Additionally, the taxonomic composition of the microbiome was only profiled, and thus cannot detect interactions that are dependent on microbial genes or functions. Similarly, using whole-exome sequencing does not allow inclusion of non-coding mutations and larger tumor structural variants and chromosomal abnormalities. This can be alleviated by the use of metagenomic shotgun sequencing to profile the microbiome, as well as whole-genome sequencing to assess tumor mutations. Moreover, the study sample was relatively small (n=88 samples from 44 patients). Nevertheless, the sample size was sufficient to detect significant patterns. Additional studies that use large tumor samples would be useful in validating our results and identifying further associations.

In summary, there is a strong association between tumor genetic profiles and the proximal microbiome, and identify tumor genes and pathways that correlate with specific microbial taxa. Moreover, the microbiome can be used as a predictor of tumor mutated genes and pathways, and suggest potential mechanisms driving the interaction between the tumor and its microbiota. The present analysis can provide a starting point for the development of diagnostics that utilize microbiome profiles to ascertain CRC tumor mutational profiles, facilitating personalized treatments.

### REFERENCES

Ahn et al., J. Nat. Cancer Inst., 15:1907 (2013).
Albert et al., Infect Immun., 0:5017 (1992).
Allali at al., Gut Microbes, 6:161 (2015).
Allen-Vercoe et al., Gut Microbes. 2:294 (2011).
Anon, Babraham Bioinformatics—FastQC A Quality Control tool for High Throughput Sequence Data. Available at http://www.bioinformatics.babraham.ac.uk/projects/fastqc/ [Accessed Sep. 28, 2015a].
Anon, Picard Tools—By Broad Institute. Available at: http://broadinstitute.github.io/picard/[Accessed Sep. 29, 2015b].
Arthur et al., Science, 38:120 (2012).
Asakura et al., Food Addit. Amo. Contam. Part A Chem. Anal. Control Expo. Risk Assess. 30:1459 (2013).
Babraham Bioirformatics—FastQC A Quality Control tool for High Throughput Sequence Data Available at: http://www.bioinformatics.babraham.ac.uk/projects/fastqc/ [Accessed Sep. 28, 2015].
Baxter et al., Genome Med., 8:37 (2016).
Berry et al., ISME J., 6:2091 (2012).
Blekhman et al., Genome Biol. 16:191 (2015).
Bonnet et al., Clin. Cancer Res. Off. J. Am. Assoc. Cancer Res., 20:859 (2014).
Boonanantanasarn et al., Infect. Immun., 80:3545 (2012).
Boutaga et al., FEMS Immunol. Med. Microbiol., 4:191 (2005).
Broad Institute Picard Tools—The Broad Institute. Available at: http://broadinstitute.github.io/picard/ [Accessed Sep. 29, 2015].
Brook and Walker, J. Med. Microbial., 21:93 (1986).
Burns et al., Genome Med., 7:55 (2015).
Burns et al., Nature, 494:366 (2013).
Cai et al., PLoS One. 8:e53649 (2013).
Cancer Genome Atlas Network, Nature. 487:330 (2012).
Caporaso et al., Nat. Methods, 7:335 (2010).
Carroll et al., Gut Pathoa., 2:19 (2010).
Castellarin et al., Genome Res., 22:299 (2012).
Chen et al., Oncol. Reo., 30:1906 (2013).
Chen et al., PLoS One, 7:e39743 (2012).
Cingolani et al., Fly, 8:80 (2012).
Cott et al., Biochim Biophys Acta., doi:10.1016/j.bbamcr.2016.02.004 (2016).
Couturier-Maillard et al., J. Clin. Invest. 12:700 (2013).
Cui et al., Int. J. Cancer, 137:86 (2015).
Cullender et al., Cell Host Microbe. 14:571 (2013).
Davenport et al., PLoS One, 10:e0140301 (2015).
David et al., Nature, 505:559 (2014).
Dejea et al., Proc. Natl. Acad. Sci. USA, 111:18321 (2014).
Dennis et al., Cancer Res., 73:5905 (2013).
DeSantis et al., Appl. Environ. Microbiol. 72:5069 (2006).
Dowd et al., BMC Microbiol., 8:43 (2008).
Dutilh et al., Pract. Res. Clin. Gastroenterol., 27:85 (2013).
Edgar, Bioinformatics, 26:2460 (2010).
Fang, Acta. Biochim. Biophys. Sin., 48:27 (2016).
Flemer et al., Gut., doi:10.1136/gutjnl-2015-309595 (2016).
Friedman et al., PLoS Comput. Biol., 8:e1002687 (2012).
Galac and Lazzaro, BMC Genomics, 12:612 (2012).
Garber et al., FEMS Microbiol. Lett., 48:331 (1987).
Garrison et al., arXiv [q-bioGN]. Available at: http//arxv.org/abs/1207.3907 (2012).
Geng et al., Gut Pathog., 5:2 (2013).
Goodrich et al., Cell Host Microbe., 19:731 (2016).
Goodrich et al., Cell, 19:789 (2014).
Goodrich et al., Science. 52:532 (2016).
Guth and Perrella, J. Med. Microbiol., 45:459 (1996).
Hu et al., Carcinogenesis, doi:10.1093/carcin/bgw019 (2016).
Human Microbiome Project Consortium, Nature, 486:207 (2012).
Irrazábal et al., Mol Cell, 54:309 (2014).
Jemal et al., CA Cancer J. Clin., 61:69 (2011).
Jones et al., Gut Microbes. 5:446 (2014).
Kanehisa and Goto, Nucleic Acids Res., 28:27 (2000).
Kanehisa et al., Nucleic Acids Res., 28:27 (2000).
Kanehisa et al., Nucleic Acids Res., 42:D199 (2014).
Kanehisa et al., Nucleic Acids Res., doi:10.1093/nar/gkv1070 (2015).
Kholodkova et al., Zh Mikrobiol. Epidemiol. Immunobiol., 1:20 (1977).
Knights et al., Genome Med., 6: 107 (2014).
Knights et al., Gut, 2:1505 (2013).
Kobayashi et al., Immunity, 4:756 (2015).
Kolenbrander, Annu. Rev. Microbiol., 5:413 (2000).
Konstantinov et al., Nat Rev. Gastroenterol. Heoatol., 1:741 (2013).
Koreishi et al., Ophthalmology. 113:1463 (2006).
Kostic et al., Cell Host Microbe. 14:207 (2013).
Kostic et al., Genome Res., 22:292 (2012).
Kraemer et al., J. Biol. Chem., 289:21205 (2014).
Kumar et al., Proc. Assoc. Am. Physicians, 107:296 (1995).
Lando et al., Epigenetics. 1:970 (2015).
Langille et al., Nat Biotechnol., 1:814 (2013).
Lau et al., J. Microbiol. Immunol. Infect., 37:185 (2004).
Le Moan et al., Mol. Cell, 44:476 (2011).
Lee et al., J. Microbiol. Seoul Korea, 45:272 (2007).
Letunic and Bork, Nucleic Acids Res., 39:W475 (2011).
Li et al., Bioinformatics, 25:1754 (2009).
Li H, et al., Bioinformatics, 25:2078 (2009).
Lopes, Bioinforma Oxf Engl., 26:2347 (2010).
Louis et al., Nat. Rev. Microbiol., 12:661 (2014).
Luzzaro et al., J. Clin. Microbiol., 44:1659 (2006).
Manthey et al., Am. J. Physiol. Cell Physiol., 307:C180 (2014).
Marchesi et al., PLoS One, 6:e20447 (2011).
Martin, EMBnet. journal, 17:10 (2011).
Mima et al., Gut., doi:10.1136/gutjnl-2015-310101 (2015).
Mira-Pascual et al., J. Gastroenterol., 50:167 (2014).
Montassier et al., Genome Med., 8:49 (2016).
Murata et al., J. Infect. Dis., 184:1050 (2001).
Murphy et al., Mol. Microbiol., 94:403 (2014).
Nakatsu et al., Nat Commun., 6:8727 (2015).
Navas-Molina et al., Methods Enzymol., 31:371 (2013).
Ohigashi et al., Dig. Dis. Sci., 5:1717 (2013).
Onoue et al., Microbiol. Immunol., 40:323 (1996).
Paliwal et al., Cancer Res., 67:9322 (2007).
Pfaffl, Nucleic Acids Res., 29:e45 (2001).
Pflug et al., Cancer Res., 52:5403 (1992).
Rubinstein et al., Cell Host Microbe., 14:195 (2013).
Schaefer et al., Nucleic Acids Res., 37:D674 (2009).
Segata et al., Genome Biol., 12:R60 (2011).
Shannon et al., Genome Res., 13:2498 (2003).
Shen et al., Gut Microbes., 1:138 (2010).
Shima et al., Infect. Immun., 80:1323 (2012).
Shima et al., Jon. J. Infect. Dis., 65:545 (2012).
Sing et al., Bioinformatics, 21:3940 (2005).
Sobhani et al., PLoS One, 6:e16393 (2011).
Song et al., Immunity, 40:140 (2014).
SparCC. https://bitbucket.org/yonatanf/sparcc.
Tahara et al., Cancer Res., 74:1311 (2014).
The Human Microbiome Consortium, Nature, 486:207 (2012).
Tsuruya et al., Alcohol Alcohol, doi:10.1093/alcalc/agv135 (2016).
Uniprot. http://www.uniprot.org.
Vandeventer et al., J. Clin. Microbiol., 492533 (2011).
Wang et al., Clin. Exo. Metastasis, 32:73 (2015).
Wang et al., ISME J., 6:320 (2012).
Warren et al., Microbiome, 1:16 (2013).
Weaver et al., Gut, 29:1539 (1988).
Weir et al., PLoS One. 8:e70803 (2013).
Wu et al., Microb. Ecol., 66:462 (2013).
Wynendaele et al., Peptides, 64:40 (2015).
Yamaguchi et al., Int. J. Oncol., 48:1943 (2016).
Yamaguchi et al., World J. Surg. Oncol., 14:40 (2016).
Yan et al., Am. J. Cancer Res., 5:3111 (2015).
Yang et al., Eur. J. Pharmacol., 714:120 (2013).
Yatsunenko et al., Nature, 486:222 (2012).
Yoh et al., J. Med. Microbiol., 54:1077 (2005).
Yu et al., Arch. Med. Sci., 11:385 (2015).
Yu et al., Gut., doi:10.1136/gutjnl-2015-309800 (2015).
Zackular et al., Cancer Prev. Res. (Phila), 7:1112 (2014).
Zeller et al., Mol. Syst. Biol., 10:766 (2014).
Zhang et al., J. Microbiol., 53:398 (2015).
Zhou et al., Nucleic Acids Res., 35:D391 (2007).
Zhu et al., PLoS One, 9:e90849 (2014).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to detect colon cancer in a human, comprising:
providing a tissue sample or a stool sample from a human;
analyzing the sample for bacteria including one or more of Actinobacteria OPB41; Saprospiracese; Capnocytophaga; Christensenellaceae; class ABY1; Chthoniobacteraceae; Acidobacteria BPC102, B110; Acidomicrobiales; Corynbacterium; Collinesia; Rhodocyclaceae; TM7; Thermogymnononas; Cryomorphaceae; Gemmatimonadetes; Sphingomondaceae; Moraxellaceae; class Verruco_5 order SS1B 03 39; Haptophyceae; Alphaproteobacteria; Rhizobiaceae; or Chromatiales; and
determining whether the human has colon cancer, wherein an increase in the relative amount of the one or more bacteria in the sample is indicative of colon cancer in the human.

2. The method of claim 1 wherein the sample is analyzed using a nucleic acid amplification reaction.

3. The method of claim 1 wherein the analyzing includes detecting family, order-, class- and/or genus-specific 16S rRNA.

4. The method of claim 1 wherein the analyzing includes hybridizing bacterial nucleic acid in the sample to beads or to an array.

5. The method of claim 1 wherein a plurality of the bacteria are detected.

6. The method of claim 1 wherein the sample is analyzed for nucleic acid of the bacteria using genome sequencing.

7. The method of claim 1 wherein the sample is analyzed for one or more of Actinobacteria OPB41; Capnocytophaga; Christensenellaceae; class ABY1; Acidobacteria BPC102, B110; Acidomicrobiates; Corynbacterium; Collenesia; Rhodocyclaceae; Chthoniobactereceae DA101; Thermogymnononas; Gemmatimonadetes; Sphingomondaceae; Moraxellaceae; class Verruco_5 order SS1B 03 39; Haptophyceae; or Rhizobiaceae.

8. A method to predict a loss of function (LoF) mutation in an adenomatous polyposis coli (APC) gene, a zinc finger nuclease 717 (ZFN717) gene, an ankyrin repeat domain (ANKRD) gene, a C-terminal binding protein (CTPB2) gene, or a lysine specific methyltransferase 2C (KMT2C) gene in a sample from a human, comprising:
providing a tissue sample or a stool sample from a human;
analyzing the sample for bacteria including one or more of Actinobacteria OPB41; Saprospiracese; Capnocytophaga; Christensenellaceae; class ABY1; Chthoniobacteraceae; Acidobacteria BPC102, B110; Acidomicrobiales; Corynbacterium; Collinesia; Rhodocyclaceae; TM7; Thermogymnononas; Cryomorphaceae; Gemmatimonadetes; Sphingomondaceae; Moraxellaceae; class Verruco_5 order SS1B 03 39; Haptophyceae; Alphaproteobacteria; Rhizobiaceae; or Chromatiales; and
determining whether the sample has cells with the LoF mutation, wherein an increase in the relative amount of the one or more bacteria in the sample is indicative of a LoF mutation in the APC, ZNF717, ANKRD, CTPB2 or KMT2C gene in the cells.

9. The method of claim 8 wherein the sample is analyzed using a nucleic acid amplification reaction.

10. The method of claim 8 wherein the analyzing includes detecting family-, order-, class- and/or genus-specific 16S rRNA.

11. The method claim 8 wherein the analyzing includes hybridizing bacterial nucleic acid in the sample to heads or to an array.

12. The method of claim 8 wherein a plurality of the bacteria are detected.

13. The method of claim 8 wherein the human is determined to have has an increased risk of an APC LoF mutation.

14. The method of claim 13 wherein the sample is analyzed for one or more of Actinobacteria OPB41; Capnocytophaga; or class ABY1 or analyzed for one or more of Saprospiraceae; Christensenellaceae; Rhodocyclaceae; or Chthoniobacteraceae.

15. (canceled)

16. The method of claim 8 wherein the human is determined to have an increased risk for a ZFN717 LoF mutation.

17. The method of claim 16 wherein the sample is analyzed for one or more of Acidobacteria BPC102, B110; Acidomicrobiates; Corynbacterium; Collenesia; Christensenellaceae; Rhodocyclaceae; or Chthoniobactereceae DA101.

18. The method of claim 17 wherein the sample is further analyzed for TM7_1.

19. The method of claim 8 wherein the human is determined to have an increased risk of an ANRKD LoF mutation.

20. The method of claim 19 wherein the sample is analyzed for one or more of Thermogymnomonas; Gemmatimonadetes; Sphingomonadaceae; Moraxellaceae; or class Verruco_5 order SS1B 03 39; analyzed for Cryomorphaceae analyzed for one or more of Haptophyceae or Rhizobiaceae; analyzed for Alphaproteobacteria; analyzed for Actinobacteria OPB41, Capnocytophaga, class ABY1, Saprospiraceae, Christensenellaceae, Rhodocyclaceae, and Chthoniobacteraceae; analyzed for Acidobacteria BPC102, B110, Acidomicrobiates, Corynbacterium, Collenesia, Christensenellaceae, Rhodocyclaceae, Chthoniobactereceae DA101, and TM7_1; analyzed for Thermogymnomonas, Gemmatimonadetes, Sphingomonadaceae, Moraxellaceae, class Verruco_5 order SS1B 03 39, and Cryomorphaceae; analyzed for Haptophyceae, Rhizobiaceae and Alphaproteobacteria, or any combination thereof.

21-28. (canceled)

29. A kit comprising:
a set of probes or primers to detect one or more of a) Actinobacteria OPB41; Saprospiroceae; Capnocytophaga; Christensenellaceae; phylum OD1 class ABY1; Rhodocyclaceae; or Chthoniobacteraceae DA101; b) Acidobacteria BPC102, B110; Acidomicrobiates, Corynbacterium, Collenesia, Christensenellaceae, Rhodocyclaceae, phylum TM7 class TM7_1 or Chthoniobactereceae DA101; c) Thermogymnomonas, Cryomorphaceae, Gemmatimonadetes, Sphingomonadaceae, Moraxellaceae or class Verruco_5 order SS1B 03 39; d) Haptophyceae, Alphaproteobacteria or Rhizobiaceae; or e) Chromatiales.

30-31. (canceled)


SEQUENCE LISTING (8 sequences)

SEQ ID NO: 1 - DNA, 20 bp, Artificial Sequence
  A synthetic primer
gaagaaagagcacaagctga

SEQ ID NO: 2 - DNA, 20 bp, Artificial Sequence
  A synthetic primer
gcttgaagtctttgagctct

SEQ ID NO: 3 - DNA, 17 bp, Artificial Sequence
  A synthetic primer
ggtgaatacgttcccgg

SEQ ID NO: 4 - DNA, 22 bp, Artificial Sequence
  A synthetic primer
tacggctaccttgttacgactt

SEQ ID NO: 5 - DNA, 20 bp, Artificial Sequence
  A synthetic primer
ggatttattgggcgtaaagc

SEQ ID NO: 6 - DNA, 24 bp, Artificial Sequence
  A synthetic primer
ggcattcctacaaatatctacgaa

SEQ ID NO: 7 - DNA, 18 bp, Artificial Sequence
  A synthetic primer
accgcataatctcttagg

SEQ ID NO: 8 - DNA, 18 bp, Artificial Sequence
  A synthetic primer
ctacacatggaattctac